(19) United States
(12) Patent Application Publication
(10) Pub. No.: US 2010/0304998 A1
(43) Pub. Date: Dec. 2, 2010

(54) Chemical Proteomic Assay for Optimizing Drug Binding to Target Proteins

(75) Inventor: Daniel S. Sem, New Berlin, WI (US)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: MARQUETTE UNIVERSITY, Milwaukee, WI (US)

(21) Appl. No.: 12/792,398

(22) Filed: Jun. 2, 2010

Related U.S. Application Data
(60) Provisional application No. 61/217,585, filed on Jun. 2, 2009.

(51) Int. Cl. G01N 33/545; G01N 27/26; C40B 30/04

(52) U.S. Cl. 506/9; 436/531; 204/456; 435/7.1

(57) ABSTRACT

Disclosed herein are methods related to drug development. The methods typically include steps whereby an existing drug is modified to obtain a derivative form or whereby an analog of an existing drug is identified in order to obtain a new therapeutic agent that preferably has a higher efficacy and fewer side effects than the existing drug.

### CROSS-REFERENCE TO RELATED APPLICATIONS

The present application claims the benefit under 35 U.S.C. §119(e) to U.S. Provisional Application No. 60/217,585, filed on Jun. 2, 2009, the contents of which are incorporated herein by reference.

### BACKGROUND

The field of the present invention relates to drug development. In particular, the invention relates to methods for modifying or repurposing existing drugs to obtain a new therapeutic having higher efficacy and fewer side effects.

The drug discovery process is costly and often inefficient. Genomics and proteomics advances have presented the promise of improving efficiency, but this has largely translated into the identification of new drug targets, not new drugs. What is needed is a better coupling of the chemistry of drug design to advances in genomics and proteomics.

Drugs typically exert their desired therapeutic effects and their undesired side effects by virtue of binding interactions with protein target(s) and anti-target(s), respectively. Better strategies are therefore needed to efficiently monitor and manipulate cross-target binding profiles (i.e. the collection of proteins that a drug molecule binds to), as an integrated part of the drug design process. Notably, it was only recently discovered that two widely-used drugs, imatinib and isoniazid, actually bind to multiple proteins. This was only discovered years after these drugs were in use. It should also be noted that drug binding to other proteins, while sometimes leading to toxic side-effects, can in some cases, such as imatinib (4-[(4-Methyl-1-piperazinyl)methyl]-N-[4-methyl-3-[[4-(3-pyridinyl)-2-pyrimidinyl]amino]-phenyl]benzamide methanesulfonate) and isoniazid, actually contribute to drug efficacy, thereby calling into question the one-target/one-drug dogma that has long served as the foundation for rational drug design.

The methods disclosed herein may be utilized to define proteomic profiles early in the drug discovery process. As such, lead drugs may be modified in order to tune or adjust these proteomic profiles. The methods disclosed herein also may be utilized to assay for off-target binding events, for example, so that multi-target binding can be better correlated with desired therapeutic effects.

### SUMMARY

Disclosed herein are methods related to drug development. The methods typically include steps whereby an existing drug is modified to obtain a derivative form or whereby an analog of an existing drug is identified in order to obtain a new therapeutic agent which preferably has a higher efficacy and fewer side effects than the existing drug. In some embodiments, the existing drug is utilized as an affinity agent in order to identify proteins in a biological sample that bind to the existing drug, including a target protein and optionally a non-target protein. A derivative or analog of the existing drug then is tested in order to determine: (1) whether the derivative or analog preferably has an affinity for the target protein that is no less than the affinity of the existing drug for the target protein; and optionally (2) whether the derivative or analog preferably has an affinity for the non-target protein that is less than the affinity of the existing drug for the target protein.

In some embodiments, the methods include the following steps: (a) passing a biological sample comprising a target protein and optionally a non-target protein over a column, the column comprising an affinity resin for the target protein, the affinity resin comprising a resin conjugated or covalently attached to a first chemical compound that binds to the target protein; (b) washing the column and removing proteins that are not bound to the affinity resin; (c) eluting proteins from the column that are bound to the affinity resin by passing a solution comprising a second chemical compound over the column; and (d) identifying proteins in the eluate and optionally obtaining a proteomic profile for the second chemical compound. Optionally, the methods further may include: (e) comparing the identified proteins of the eluate obtained using the second chemical compound to identified proteins of an eluate obtained using the first chemical compound (e.g., comparing the proteomic profile of the second chemical compound to the proteomic profile of the first chemical compound.

The first and second chemical compounds utilized in the method may be related or unrelated. In some embodiments, the second chemical compound is a derivative or analog of the first chemical compound and binds to the target protein. In other embodiments, the first chemical compound and the second chemical compound are selected from Table 6-9, and optionally, the second chemical compound binds to the target protein.

In some embodiments of the disclosed methods, the first chemical compound is an existing drug for which a target protein has been identified in the art and the second chemical compound is a derivative or analog of the existing drug which binds to the target protein. In the methods, the biological sample includes the target protein. Typically, the biological sample is obtained from a physiologically relevant tissue with respect to the therapeutic target of the existing drug. For example, where the existing drug is utilized as a neurological therapeutic and is known to have a target protein that is present in neural tissue, the biological sample for the present methods may be obtained from neural tissue. Where an existing drug is observed to cause side effects due to toxicity, the existing drug may be observed to bind to a non-target protein which may be present in physiologically non-relevant tissue with respect to the therapeutic target of the existing drug (e.g., non-neural tissue such as liver tissue or heart tissue for existing drugs utilized as neurological therapeutics), and which optionally may be present in physiologically relevant tissue with respect to the therapeutic target of the existing drug (e.g., neural tissue for existing drugs utilized as neurological therapeutics).

In the disclosed methods, the proteins of the biological sample are bound to the column containing the affinity resin and subsequently the proteins are eluted. For example, the proteins bound to the column may be eluted by washing the column with a solution comprising the first chemical compound (e.g., an existing drug) or a derivative or analog thereof, where the affinity resin of the first column is made of a resin conjugated or covalently attached to the first chemical compound.

In the disclosed methods, the proteins in the eluates typically are identified, for example, in order to obtain a proteomic profile. In some embodiments, the proteins in the eluates are identified by performing sodium dodecyl sulfate (SDS) polyacrylamide gel electrophoresis (PAGE). The pattern and intensity of protein bands on the gel may be compared, either visually or quantitatively, such as by performing densitometric scanning of the gel and mathematical comparison using correlation analysis. In further embodiments, the proteins in the eluates are identified by performing mass spectrometry (MS) analysis (e.g., tandem MS analysis). In some embodiments, tandem MS analysis is performed on the entire eluate or a sample thereof. In other embodiments, the eluate or a sample thereof is subjected to PAGE in order to separate proteins in the eluate, and subsequently one or more bands are excised from the gel. Then, tandem MS analysis is performed on each of the one or more bands that have been excised from the gel (e.g., in order to identify protein present in the band).

In the methods, the affinities of the first chemical compound (e.g., an existing drug) and the second chemical compound (e.g., a derivative or analog the existing drug) for the target protein and optionally the non-target protein may be compared. For example, the affinities of the first chemical compound and the second chemical compound for the target protein and the non-target protein may be compared by measuring intensities of bands in gels corresponding to the target protein and the non-target protein after performing PAGE. By performing such a comparison, the second chemical compound can be optimized such that it has a relatively high ratio of band intensity for the target band(s) versus the non-target band(s). In some embodiments: (1) the intensity of the band corresponding to the target protein in the eluate obtained by using the second compound as an eluent is no less than the intensity of the band corresponding to the target protein in the eluate obtained by using the first compound as an eluent; and optionally (2) the intensity of the band corresponding to the non-target protein in the eluate obtained by using the second compound as an eluent is less than the intensity of the band corresponding to the non-target protein in the eluate obtained by using the first compound as an eluent. The intensities of bands in gels may be measured by methods that include, but are not limited to, electronically scanning the gels and performing densitometry analysis.

Preferably, the methods are performed in order to obtain a second chemical compound that binds to the target protein with an affinity no less than the affinity of the first chemical compound and that binds to the non-target protein with an affinity less than the affinity of the first chemical compound. As such, the methods may be performed in order to obtain a second chemical compound that has an efficacy that is at least as high as the first chemical compound, and further that has fewer or less severe side effects or toxicity.

In other embodiment, the methods may include the following steps: (a) passing a biological sample comprising proteins over columns comprising a chemical-resin library, wherein each column comprises a separate member of the chemical-resin library and the chemical-resin library comprises a separate chemical compound conjugated to a resin; (b) washing each column to remove any non-bound proteins; (c) eluting any bound proteins from each column; and (d) identifying proteins in the eluates from each column, optionally generating a proteomic profile for each column. Optionally, the methods further may include (e) comparing the identified proteins in the eluates (e.g., comparing proteomic profiles).

In further embodiments, the disclosed methods include the following steps: (a) passing a biological sample including a target protein and a non-target protein over a first column, the first column containing an affinity resin for the target protein, the affinity resin made of a resin conjugated or covalently attached to a first chemical compound (e.g., an existing drug) that binds to the target protein; (b) washing the first column and removing proteins that are not bound to the affinity resin; (c) eluting proteins from the first column that are bound to the affinity resin (e.g., by washing the first column with a solution comprising the first chemical compound or a derivative or analog thereof); (d) identifying proteins in the eluate including the target protein and optionally the non-target protein; (e) passing the biological sample including the target protein and the non-target protein over a second column, the second column containing an affinity resin for the target protein, the affinity resin made of a resin conjugated or covalently attached to a second chemical compound (e.g., a derivative or analog of the existing drug) that binds to the target protein; (f) washing the second column and removing proteins that are not bound to the affinity resin; (g) eluting proteins from the second column that are bound to the affinity resin (e.g., by washing the second column with a solution comprising the second chemical compound or a derivative or analog thereof); and (h) identifying proteins in the eluate including the target protein and optionally the non-target protein. Optionally, the second chemical compound binds the target protein with an affinity no less than the first chemical compound and the second chemical compound preferably binds the non-target protein with an affinity less than the first chemical compound.

In even further embodiments, the methods may include the following steps: (a) passing a biological sample comprising a target protein and a non-target protein over a first column, the first column comprising a affinity resin for the target protein, the affinity resin comprising a resin conjugated or covalently attached to a first chemical compound that binds to the target protein; (b) washing the first column and removing proteins that are not bound to the affinity resin; (c) eluting proteins from the first column that are bound to the affinity resin; (d) identifying proteins in the eluate including the target protein and optionally the non-target protein, thereby generating a proteomic profile for the first chemical compound; (e) passing the biological sample comprising the target protein and the non-target protein over a second column, the second column comprising an affinity resin for the target protein, the affinity resin comprising a resin conjugated or covalently attached to a second chemical compound that binds to the target protein; (f) washing the second column and removing proteins that are not bound to the affinity resin; (g) eluting proteins from the second column that are bound to the affinity resin; and (h) identifying proteins in the eluate including the target protein and optionally the non-target protein, thereby generating a proteomic profile for the second chemical compound; and (i) comparing the proteomic profile of the first chemical compound and the proteomic profile of the second chemical compound; wherein the second chemical compound binds the target protein with an affinity no less than the first chemical compound and the second chemical compound binds the non-target protein with an affinity less than the first chemical compound.

### BRIEF DESCRIPTION OF THE DRAWINGS

FIG. 1. The catechol rhodanine privileged scaffold (CRAA, 1), and its use in creating bi-ligand inhibitors with high affinity and specificity for specific dehydrogenase targets.

FIG. 2. Synthesis of the NHS-CRAA active ester (2) and CRAA aminohexyl agarose matrix.

FIG. 3. (a) E. coli uptake study. Cartoon representation of the uptake study, demonstrating that NHS-CRAA ester (2) can cross the E. coli cell wall to react with overexpressed DHPR, and other intracellular proteins. (b) SDS-PAGE analysis of the crude cell lysate from the experiment in panel (a). Lane 1: protein marker; Lanes 2 and 4: lysate of cells with DHPR present (+IPTG). Lanes 3 and 5: lysate of cells without DHPR present (—IPTG). Lanes 2 and 3 were fluorescently scanned using a Kodak Image Station; Lanes 4 and 5 were scanned with a CanonScan D1250U2F document scanner after Coomassie blue staining, using with the same Gel. (c) Vials of E. coli cells just prior to lysis, showing the CRAA-associated color change in the cells containing overexpressed DHPR (left), relative to those without DHFR (right).

FIG. 4. E. coli fluorescence labeling. (a) Fluorescence and (b) bright field images of E. coli cells containing overexpressed DHPR, after incubation with NHS-CRAA (2) and subsequent washing with PBS. A 100× objective was used, and 495 nm/520 nm excitation/emission filters.

FIG. 5. Proteome fishing with a privileged scaffold. Cartoon representation of how the CRAA (1) affinity column is used for protein target fishing (e.g., affinity purification) in a proteome pool, either to initially identify potential targets and antitargets (top branch), or to later characterize the binding profile for a particular biligand drug lead candidate (bottom branch). The top branch also demonstrates how one assess whether a privileged scaffold really is targeting a gene family (as intended), such as NAD(P)(H) binding proteins.

FIG. 6. SDS-PAGE analysis of the proteome fishing experiments described in FIG. 5. (a) SDS-PAGE analysis of CRAA-captured proteins from E. coli and M. tuberculosis CH37RV proteomes. Lane 1, Protein marker; Lane 2, E. coli crude cell lysate; Lane 3, E. coli column wash; Lanes 4-6, E. coli fractions after elution with free CRAA (1). Lane 7, M. tuberculosis crude cell lysate; Lane 8, M. tuberculosis column wash; Lanes 9-11, M. tuberculosis fractions after elution with free CRAA (1). (b) SDS-PAGE analysis of CRAA-captured proteins from human liver proteome. Lane 1, Protein marker; Lane 2, crude cell lysate; Lanes 3-5, column wash; Lanes 6-12, fractions after elution with free CRAA (1). (c) SDS-PAGE analysis of human liver and M. tuberculosis CH37RV protein fractions after CRAA (1) affinity column chromatography, showing the protein bands that were cut and extracted for nanospray-LC/MS/MS proteomic analysis. Identical samples to those loaded on the gel were polymerized in a gel piece as described in the methods and subjected to whole proteomic analyses. Labeled bands A, B, C and D are referred to in Table 3 (database search results). Gels were silver stained.

FIG. 7. (a) Native gel of DHPR that has been covalently labeled (purified protein) with the NHS-CRAA active ester. Lanes represent increasing concentrations of DHPR, (b) Crystal structure (pdb code 1ARZ) of DHPR, showing proximity of lysine (163) to the NADH (left) and PDC (right) ligands.

FIG. 8. SDS-PAGE gel of DHPR that has been labeled with NHS-CRAA active ester, but in the presence of increasing concentrations of either NADH or PDC (2,6-pyridine dicarboxylic acid) competitors, Lane 1, protein standard; Lanes 2-6, increasing NADH (0, 0.057, 0.11, 0.23, 0.45 nM); Lanes 7-10, increasing PDC (1.1, 2.3, 4.5, 9.1 mM).

FIG. 9. (A) Fluorescence and (B) bright field images of E. coli cells exposed to NHS-CRAA, but were not expressing DHPR (−IPTG). (C) Overlay of (A) and (B).

FIG. 10. SDS-PAGE analysis of human liver and Mycobacterium tuberculosis proteomes after CRAA affinity column. Lane 1, Protein marker; Lane 2, Human liver crude cell lysate; Lanes 3-5, fractions 7 and 8 after elution with CRAA; Lane 6, Mycobacterium tuberculosis crude cell lysate; Lanes 7-10, fractions 7 and 8 after elution with CRAA, with two lanes for each fraction.

FIG. 11. SDS-PAGE analysis of E. coli containing overexpressed DHPR, showing the purification achieved using the CRAA affinity column (10 mL), with subsequent elution using NADH. Wash was with 40 mL of 25 mM Tris (pH 7.8). Elution was with 40 mL of 10 mM NADH in 150 mM NaCl, 25 mM Tris (pH 7.8). Lane 1, Protein marker; Lane 2, crude cell lysate; Lane 3, flow through; Lanes 4-9, elution with NADH; Lane 10, unrelated sample. Gel was stained with Coomasie blue.

FIG. 12. ¹H NMR spectra (in d6-DMSO) of the CRAA reaction for formation of the NHS ester.

FIG. 13. ¹H NMR STD (saturation transfer difference) spectra for CRAA binding to either malate dehydrogenase (A, MDH) or glutamate dehydrogenase (B).

FIG. 14. Elution of human liver proteins from the CRAA- and acetylamide-control resins using free CRAA to elute (control for FIG. 6). Resin was used either as is (with no ligand attached to the w-aminohexyl group), or after covalent addition of an acetyl group to make CH₃C(O)NH-hexyl-agarose. Gel: Lane 1, marker; Lane 2, human liver sample (before chromatography); Lanes 3-4 (unmodified ω-aminohexyl-agarose, with acetylamide ligand attached), with Lane 5 as the flow through and Lane 6 elution with CRAA. Elution was as in FIG. 6b.

FIG. 15. Drug lead molecules that bind to the KCNQ potassium ion channel.

FIG. 16. Attachment of DMP543 to affinity resin bead via an imine linkage.

FIG. 17. Alternative attachment of DMP453 to resin bead via an amide linkage.

FIG. 18. Attachment of Linopirdine to affinity resin bead via an amide linkage.

FIG. 19. Affinity and specificity of Common Ligand Mimic (CLM) and Bi-Ligand molecules for Oxireductases in pharmacofamilies 1 and 2. The CLM in this example is referred to as “catechol rhodanine acetic acid” (CRAA) in FIG. 1 and is the compound attached to resin in FIG. 2. Shown also are derivatives of the CRAA chemical compound that can be used to elute proteins bound to the affinity resin as shown schematically in FIG. 5.

FIG. 20. Alternative scaffolds, referring to FIG. 19, tethered to a pyridine dicarboxylate fragment, which may be used in place of CRAA as chemically attached to resin in FIG. 2.

FIG. 21. The glitazole scaffold, which is present in Actos and Avandia brand name drugs, attached to resin.

### DETAILED DESCRIPTION

Disclosed herein are methods related to drug development. The methods may be described using several definitions as discussed below.

Unless otherwise specified or indicated by context, the terms “a”, “an”, and “the” mean “one or more.” In addition, singular nouns such as “target protein” and “non-target protein” should be interpreted to mean “one or more target proteins” and “one or more non-target proteins,” unless otherwise specified or indicated by context.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising.”

A “biological sample” as used herein means any solid or liquid material that includes a target protein. A biological sample may include material obtained from an animal (e.g., human) or a non-animal source (e.g., bacteria, mycobacteria, and fungi). A biological sample may include a human biological sample, which may include but is not limited to, neurological tissue (e.g., brain), liver tissue, heart tissue, breast tissue, kidney tissue, lung tissue, and muscle tissue. A biological sample may include human body fluids (e.g., blood or blood products).

The term “proteome” as used herein refers to a complex protein mixture obtained from a biological sample. Preferred proteomes comprise at least about 5% of the total repertoire of proteins present in a biological sample preferably at least about 10%, more preferably at least about 25%, even more preferably about 75%, and generally 90% or more, up to and including the entire repertoire of proteins obtainable from the biological sample. The proteome will be a mixture of proteins, generally having at least about 20 different proteins, usually at least about 50 different proteins and in most cases 100 different proteins or more.

A “target protein” as used herein is a protein to which an existing drug or chemical compound binds, thereby modulating biological activity of the protein and causing a therapeutic effect. An “anti-target” or “non-target” is a protein to which an existing drug or chemical compound binds, thereby modulating biological activity of the protein and causing an undesirable side effect. For example, target proteins useful for the methods disclosed herein may include target proteins that are therapeutic targets for treating psychiatric disorders. Suitable target proteins include the proteins that form the KCNQ (Kv7) ion channel in neural tissue of human. The “KCNQ channels” alternatively referred to as the “Kv7 channels” are a small family of voltage-gated potassium channel subunits that are encoded by the KCNQ genes (KCNQ1-5). (See, e.g., Robbins, J. (2001). Pharmacol. Ther. 90, 1-19; and Jentsch T. J. (2000) Nat. Rev. Neurosci. 1, 21-30, the contents of which are incorporated by reference in their entireties). Modulation of KCNQ channel activity has been suggested to have therapeutic potential. (See, e.g., Wulff et al., Nature Reviews, Drug Discovery, Volume 8, Pages 982-1001, December 2009; Brown, J. Physiol. 586.7 (2008) pp 1781-1783; Gribkoff, Expert Opin. Ther. Targets (2008) 12(5):565-581; Xiong et al., Trends in Pharmacological Sciences, 2007, 29(2), pages 99-107; and Gribkoff, Expert Opin. Ther. Targets (2003) 7(6):737-748; the content of which is incorporated herein by reference in their entireties).

The methods disclosed herein may be utilized to define proteomic profiles early in the drug discovery process. “Proteomic profile” refers to the collection of proteins that a drug binds to, which leads to its desirable therapeutic properties (i.e., due to binding to the target proteins) as well as undesirable side effects (i.e., due to binding to the anti-target proteins or non-target proteins). As such, lead drugs may be modified in order to tune or adjust these proteomic profiles so there is more binding to target proteins, and less binding to anti-target proteins or non-target proteins. The methods disclosed herein may be utilized to assay for such off-target binding events, to minimize side effects of drugs. Furthermore, if a drug is exhibiting desirable properties (ex. killing Mycobacterium tuberculosis or cancer cells), by virtue of the binding interactions it has with its target protein or proteins, one can expect that another chemical that binds to these same proteins (i.e., has the same proteomic profile) might also have the same advantageous properties as that first drug. The methods disclosed herein also may be utilized to assay for such chemicals, which themselves might serve as alternatives or improvements to the first drug. If the drug binds to multiple targets (as does imatinib), once can correlate this multi-target binding with the desired therapeutic effects.

Existing drugs and chemical compounds that may be utilized in the methods disclosed herein include those drugs available from commercial libraries such as The Prestwick Chemical Library® collection (Prestwick Chemical, Inc.) (See Table 6.) Other existing drugs and chemical compounds that may be utilized in the methods disclosed herein include those drugs available from The Spectrum Collection (Microsource Discovery System, Inc.). (See Table 7. See also J. Virology 77:10288 (2003) and Ann. Rev. Med. 56:321 (2005), the contents of which are incorporated herein by reference in their entireties). Other existing drugs and chemical compound that may be utilized in the method disclosed herein include those drugs available from the Sequoia collection at its website or those drugs published by Advanstart Medical Economics: Top 200 Drugs, A 5-Year Compilation (2009), the contents of which are incorporated by reference herein in their entireties. (See Table 8).

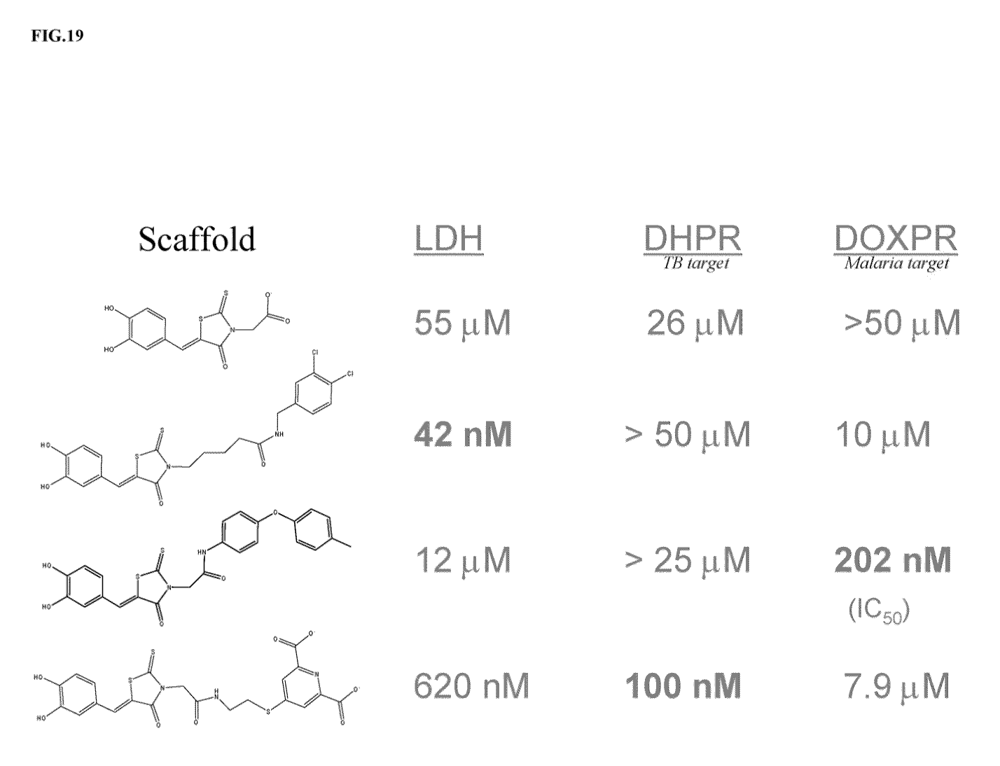

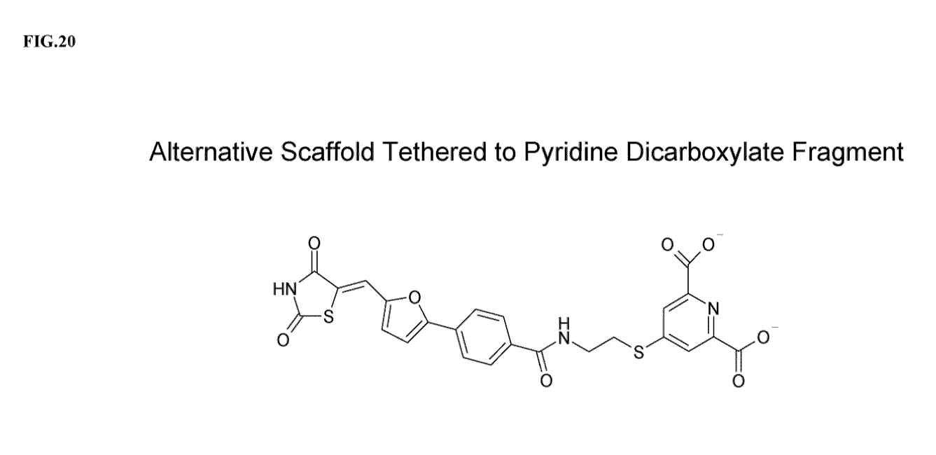

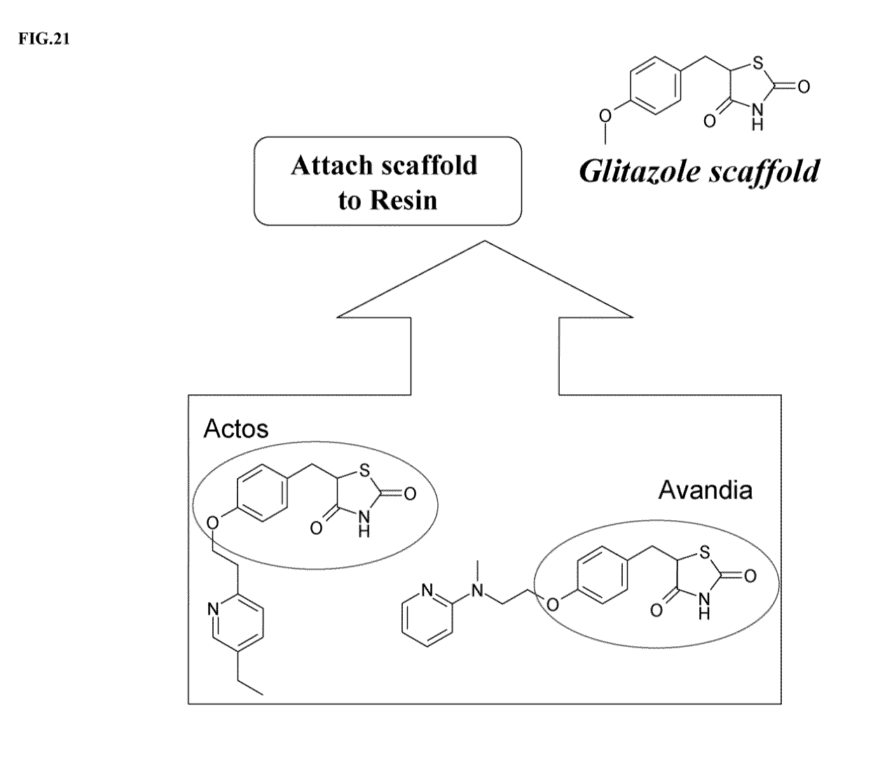

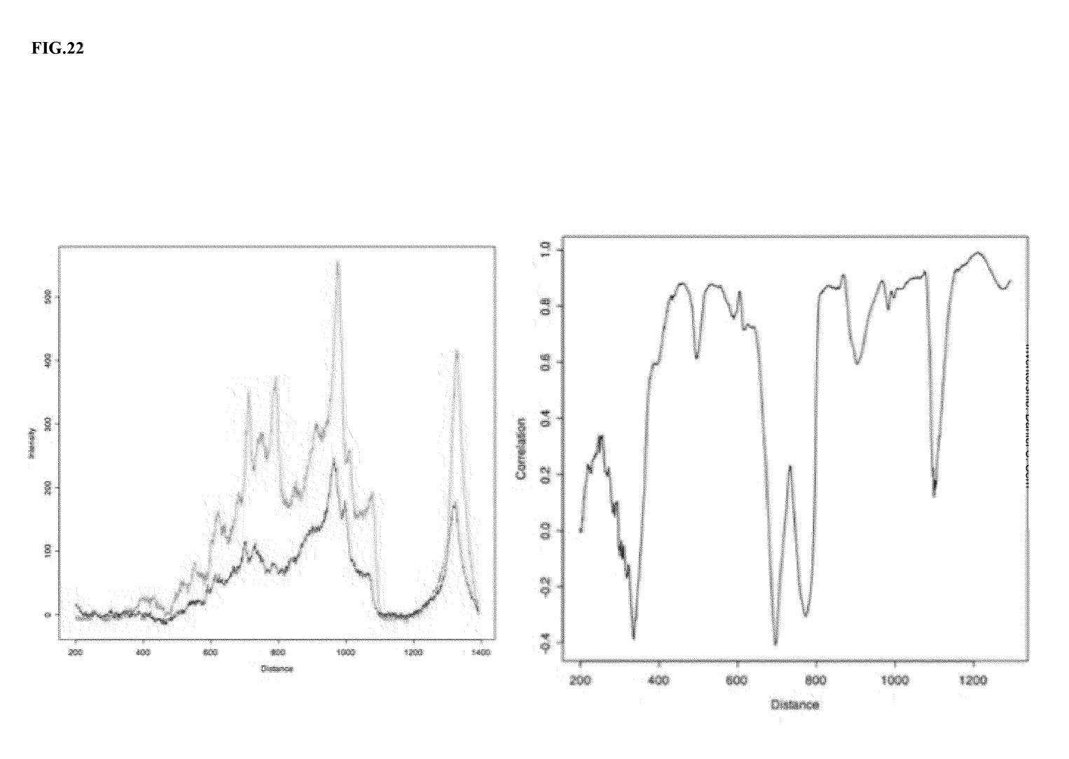

The chemical compounds utilized in the methods disclosed herein may comprise, consist essentially of, or consist of a “drug scaffold.” As used herein, a “drug scaffold” is defined as a chemical substructure common to two or more active drugs for the same disease and comprising at least two organic ring systems. Such motifs can be difficult to identify by manual inspection, so cheminformatic software can be used, such as SAR Vision (Altoris, San Diego, Calif.). An example of a drug scaffold is the glitazone scaffold contained in the two distinct diabetes drugs Actos and Avandia, which bind to the same target protein “PPAR-gamma”. Such scaffolds, if they confer modest binding affinity to more than one protein in a family are termed privileged scaffolds, because they are the starting point for building a drug to a specific target. That is, by making small chemical additions to the privileged scaffold, one can tune binding affinity to a desired target protein in the family. One such scaffold is the catechol rhodanine, and another closely related scaffold is the thiazolidinedione (Sem et al. (2004) Chem. Biol. 11, 185). The chemical linkage of the scaffold and another chemical fragment creates what is called a “bi-ligand” inhibitor, so-called because it is comprised of two ligands that are tethered (i.e., chemically and covalently joined). Suitable drug scaffolds for the methods presented herein are listed in Table 9. Other suitable scaffolds and attachment strategies are illustrated in FIGS. 19-21. Other suitable scaffolds (referred to as “privileged scaffolds” are described in Welsch et al. (2010), Current Opinions in Chemical Biology 14, 347-361, the content of which is incorporated herein by reference in its entirety.

Suitable existing drugs or chemical compounds for the methods contemplated herein may modulate KCNQ (Kv7) channel activity. These include compounds that bind to the KCNQ (Kv7) channel and inhibit or alternatively activate or enhance KCNQ (Kv7) channel activity. Suitable compounds may inhibit KCNQ (Kv7) channel activity by blocking, closing, or otherwise inhibiting a KCNQ (Kv7) channel from facilitating passage of ions from one side of a membrane to the other side of the membrane in which the KCNQ (Kv7) channel is present. KCNQ (Kv7) channel activity and modulation thereof, including inhibition thereof, may be assessed by methods described in the art (e.g., patch clamp analysis, see, e.g., Bal et al., J. Biol. Chem. 2008 283(45):30668-30676; Wu et al., J. Neurophysiol. 2008 100(4):1897-1908; Kasten et al., J. Physiol. 2007 584(Pt. 2):565-582; Jia et al., J. Gen Physiol. 2006 131(6):575-587; and Wladyka et al., J. Physiol. 2006 575(Pt. 1):175-189; the contents of which are incorporated by reference in their entireties).

Compounds that modulate KCNQ (Kv7) channel activity are known in the art and may include KCNQ (Kv7) channel activity inhibitors or alternatively KCNQ (Kv7) channel activity activators. KCNQ (Kv7) channel activity inhibitors may include but are not limited to linopirdine (Dupont), XE991 (Dupont), DMP543 (Dupont), d-tubocurarine, verapamil, 4-aminopurine, CP-339818 (Pfizer), UK-78282 (Pfizer), correolide (Merck), PAP-1 (UC-Davis), clofazimine, Icagen (Eli Lilly), AVE-0118 (Sanofi-Aventis), Vernakalant (Cardiome), ISQ-1 (Merck), TAEA (Merck), DPO-1 (Merck), azimilide (Proctor and Gamble), MHR-1556 (Sanofi-Aventis), L-768673 (Merck), astemizole, imipramine, dofetilide, NS1643 (Neurosearch), NS3623 (Neurosearch), RPR26024 (Sanofi-Aventis), PD307243 (GlaxoSmithKline), and A935142 (Abbott Laboratories). KCNQ (Kv7) channel activity activators may include but are not limited to retigabine, flupirtine, ICA-27243 (Icagen), ICA-105665 (Icagen), diclofenac, NH6, niflumic acid, mefenamic acid, and L364373 (Merck). These compounds and other compounds that modulate KCNQ (Kv7) channel activity are disclosed in Wulff et al., Nature Reviews, Drug Discovery, Volume 8, Pages 982-1001, December 2009 (the content of which is incorporated herein by reference in its entirety).

A suitable drug or compound for the methods contemplated herein may include DMP543 or analogs or derivatives thereof (e.g., analogs or derivatives thereof that inhibit KCNQ (Kv7) channel activity). Referring to the PubChem Database provided by the National Center for Biotechnology Information (NCBI) of the National Institute of Health (NIH), DMP543 is referenced by compound identification (CID) number 9887884 (which entry is incorporated herein by reference in its entirety). (See also FIG. 15.) Analogs or derivative of DMP543 may include salts, esters, amides, or solvates thereof. Furthermore, analogs or derivatives of DMP543 may include “similar compounds” or “conformer compounds” as defined at the PubChem Database, which include but are not limited to compounds referenced by CID Nos.: 9801773, 10644338, 9930525, 19606104, 10926895, 10093074, 10093073, 45194349, 19606090, 19606069, 19606087, 19606071, 19606104, 19606084, 19606108, 19606110, 19606109, and 15296110, which entries are incorporated herein by reference in their entireties. In methods where DMP543 is utilized as a compound in an affinity resin, DMP543 may be conjugated or covalently attached to the resin as follows:

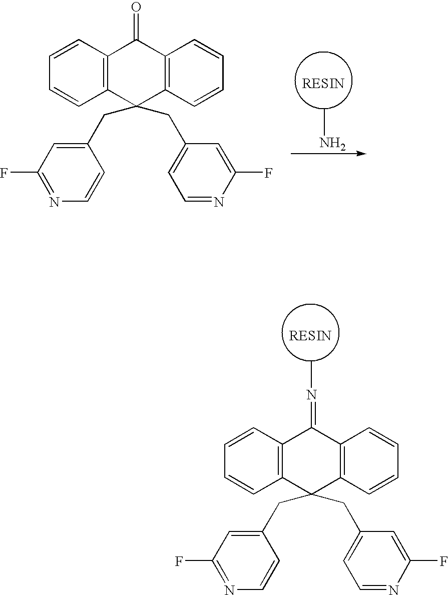

Optionally, the above-presented imine linkage can be reduced to a more stable amide linkage using, for example, sodium borohydride, sodium cyanoborohydride, or other reducing agents. Alternatively, DMP543 may be conjugated or covalently attached to the resin as follows:

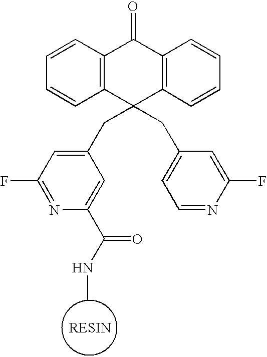

A suitable drug or compound for the methods contemplated herein may include XE991 or analogs or derivatives thereof (e.g., analogs or derivatives thereof that inhibit KCNQ (Kv7) channel activity). Referring to the PubChem Database provided by the National Center for Biotechnology Information (NCBI) of the National Institute of Health (NIH), XE991 is referenced by compound identification (CID) number 656732 (which entry is incorporated herein by reference in its entirety). (See also FIG. 15.) Analogs or derivative of XE991 may include salts, esters, amides, or solvates thereof. Furthermore, analogs or derivatives of XE991 may include “similar compounds” or “conformer compounds” as defined at the PubChem Database, which include but are not limited to compounds referenced by CID Nos.: 45073462, 17847140, 11122015, 19922429, 19922428, 15678637, 328741, 45234820, 45053849, 45053848, 42194630, 42194628, 21537929, 19922433, 14941569, 15678632, and 409154, which entries are incorporated herein by reference in their entireties. In methods where XE991 is utilized as a compound in an affinity resin, XE991 may be conjugated or covalently attached to the resin as follows:

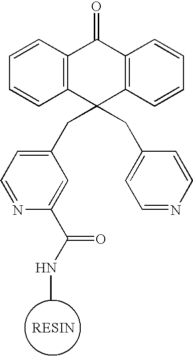

A suitable compound for the methods contemplated herein may include linopirdine or analogs or derivatives thereof (e.g., analogs or derivatives thereof that inhibit KCNQ (Kv7) channel activity). Referring to the PubChem Database provided by the National Center for Biotechnology Information (NCBI) of the National Institute of Health (NIH), linopirdine is referenced by compound identification (CID) number 3932 (which entry is incorporated herein by reference in its entirety). (See also FIG. 15.) Analogs or derivative of linopirdine may include salts, esters, amides, or solvates thereof. Furthermore, analogs or derivatives of linopirdine may include “similar compounds” or “conformer compounds” as defined at the PubChem Database, which include but are not limited to compounds referenced by CID Nos.: 11015296, 10993167, 454643, 454641, 45114239, 23581818, 14209557, 14209555, 14209553, 10549571, 9832106, 14209556, 10764944, 454654, 19438999, 14960217, 14209554, 11823673, 14209559, 15284399, 19438967, 19438958, 19438948, 19438961, 9865313, 19104987, 15296097, 19438997, 15346939, 11823673, 15284397, 15296101, 15284414, and 10476777, which entries are incorporated herein by reference in their entireties. In Methods where linopirdine is utilized as a compound in an affinity resin, linopirdine may be conjugated or covalently attached to the resin as follows:

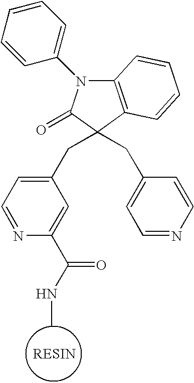

The existing drugs and compound utilized in the present methods typically are covalently attached or conjugated or covalently attached to a resin in order to generate an affinity resin suitable for use in column chromatography. Suitable resins may include, but are not limited to, agarose, acrylamide, and cellulose resin or beads which are derivatized to include a reactive group. Suitable reactive groups may include amine-reactive groups and carbonyl-reactive groups. Amine-reactive groups may include isothiocyanate groups, carboxyl groups, succinimidyl ester groups, and sulfonyl groups. Carbonyl-reactive groups may include amino groups and hydrazide. Suitable resins for attaching chemical molecules include resins containing amino groups, cyanogen bromide groups, and epoxide groups, such as resins sold by Sigma Corp. and Bio-Rad Inc. For example, a glitazole scaffold may be attached to a resin containing an epoxide group where the phenolic oxygen of the glitazole attacks the epoxide of the resin thereby attaching glitazole to the resin.

The drugs and compounds may be covalently attached or conjugated to a resin via a reactive group present on the drug or compound. Suitable reactive groups may include amine-reactive groups and carbonyl-reactive groups. Amine-reactive groups may include isothiocyanate groups, carboxyl groups, succinimidyl ester groups, and sulfonyl groups. Carbonyl-reactive groups may include amino groups and hydrazide.

Also contemplated herein are chemical-resin libraries for use in the presently disclosed methods. A chemical-resin library may be prepared by covalently attaching or conjugated a panel of chemical compounds to a resin. A panel typically will comprise at least about 5, 10, 50, 100, 200, 300, 400, or 500 chemical compounds. A chemical-resin library typically will comprise at least about 5, 10, 50, 100, 200, 300, 400, or 500 chemical compounds which are separately conjugated or covalently attached to a resin.

In the disclosed methods, proteins that bind the affinity resin are eluted and identified. The proteins may be identified by methods that include, but are not limited to, performing sodium dodecyl sulfated (SDS) polyacrylamide gel electrophoresis (PAGE) (including two-dimensional PAGE), mass spectroscopy (MS) (e.g., tandem MS), amino acid sequencing, and immunoanalysis. (See, e.g., Gevaert et al., Electrophoresis 2000 April; 21(6):1145-54, the content of which is incorporated by reference in its entirety).

The present methods may be utilized in order to identify new purposes for existing drugs, otherwise referred to as “repurposing.” Repurposing and methods for performing repurposing have been described. (See, e.g., Chong and Sullivan, Nature, Vol. 448, 9 Aug. 2007, 645-646; Keiser et al., Nature, Vol. 462, 12 Nov. 2009, 175-182; and O'Connor and Roth, Nature Reviews Drug Discover, Vol. 4, December 2005, 1005-1014; the contents of which are incorporated herein by reference in their entireties). For example, two existing compounds may be utilized in the present methods, namely a first chemical compound utilized as a known therapeutic purpose and a second chemical compound unknown for the therapeutic purpose of the first chemical compound. The present methods may be practiced in order to determine whether the second chemical compound is useful for the same therapeutic purpose as the first chemical compound by performing the following steps: (a) passing a biological sample comprising a target protein and optionally a non-target protein over a column, the column comprising an affinity resin for the target protein, the affinity resin comprising a resin conjugated or covalently attached to the first chemical compound which binds to the target protein; (b) washing the first column and removing proteins that are not bound to the affinity resin; (c) eluting proteins from the column that are bound to the affinity resin by passing a solution comprising a second chemical compound over the column; (d) identifying proteins in the eluate (i.e., generating a proteomic profile for the second chemical compound) and comparing the identified proteins to proteins eluted from the column by a solution comprising the first chemical compound (i.e., comparing the proteomic profile for the second chemical compound to the proteomic profile for the first chemical compound). Where the proteins eluted from the column by a solution comprising the second chemical compound are similar or identical to the proteins eluted from the column by the first chemical compound, the second chemical compound may be suitable for the therapeutic purpose of the first chemical compound.

Herein are presented experimental methods to identify chemicals for repurposing, based on identifying similarities in proteomic profiles. Interest in repurposing has increased, based on recent repurposed drugs such as Revlimid™ (Celgene) and Savella™ (Cypress). A drug may be repurposed by optimizing binding to what are considered non-target proteins for disease #1, but what are considered target proteins for disease #2. For example, sildenifafil (Viagra) was initially designed to be an anti-angina drug, but the side effect of producing penile erection in healthy volunteers (due to non-target binding) led to its use for erectile dysfunction. Other examples, including use of an anti-psychotic drug for treating bacterial infections. (See O'Connor and Roth, Nature Reviews Drug Discover, Vol. 4, December 2005, 1005-1014; the content of which is incorporated herein by reference in its entirety). The methods presented herein provide an experimental way to identify drugs with those unknown effects, based on their showing similar proteomic profiles to other drugs.

Utilizing a relevant biological sample in view of the therapeutic purpose of a first chemical compound, a proteomic profile may be generated for the first chemical compound. For example, where the first chemical compound is utilized for a neurological therapeutic purpose, a proteomic profile may be generated for the first chemical compound from a biological sample of neurological tissue by: (a) passing a biological sample of neurological tissue through a first column, the first column containing an affinity resin made of a resin conjugated or covalently attached to the first chemical compound (e.g., an existing drug); (b) washing the first column and removing proteins that are not bound to the affinity resin; (c) eluting proteins from the first column that are bound to the affinity resin; (d) identifying proteins in the eluate, thereby generating a proteomic profile for the first chemical compound. Having generating the proteomic profile for the first chemical compound, a second chemical compound can be identified having a similar proteomic profile by: (e) passing the biological sample of neurological tissue over a second column, the second column containing an affinity resin made of a resin conjugated or covalently attached to a second chemical compound (e.g., another existing drug); (f) washing the second column and removing proteins that are not bound to the affinity resin; (g) eluting proteins from the second column that are bound to the affinity resin; and (h) identifying proteins in the eluate, thereby generating a proteomic profile for the second chemical compound. The proteomic profiles for the first and second chemical compound may be compared. Preferably, the second chemical compound exhibits a similar proteomic profile and binds one or more target proteins with an affinity no less than the first chemical compound and optionally the second chemical compound binds one or more non-target protein with an affinity less than the first chemical compound.

Where a first chemical compound is known to exhibit side effects or toxicity when utilized as a drug, for example liver toxicity, a proteomic profile for the first chemical compound may be generated from a biological sample of liver tissue. A second chemical compound may be identified utilizing the methods herein in order to obtain a drug exhibiting fewer side effects or toxicity, for example where a proteomic profile for the second chemical compound is generated from a biological sample of liver tissue and the second chemical compound binds fewer proteins in the biological sample of liver tissue than the first chemical compound.

The disclosed methods may utilize a chemical-resin library for repurposing an existing drug by performing the following steps: (a) passing a biological sample comprising proteins over columns comprising the chemical-resin library, wherein each column comprises a separate member of the chemical-resin library; (b) washing each column to remove any non-bound proteins; (c) eluting any bound proteins from each column; (d) identifying proteins in the eluates, thereby generating a proteomic profile for each column. The proteins may be eluted, for example, by a solution comprising a chemical compound that corresponds to the compound of the chemical-resin. Where two columns exhibit a similar proteomic profile (i.e., where the proteins in the eluate from two columns are similar or identical), the two chemical compounds corresponding to the chemical-resins for the columns may be identified as having the same therapeutic purpose.

### ILLUSTRATIVE EMBODIMENTS

The following embodiments are illustrative and not intended to limit the claimed subject matter.

### Embodiment 1

A method comprising: (a) passing a biological sample comprising a target protein and optionally a non-target protein over a column, the column comprising an affinity resin for the target protein, the affinity resin comprising a resin conjugated or covalently attached to a first chemical compound that binds to the target protein; (b) washing the column and removing proteins that are not bound to the affinity resin; (c) eluting proteins from the column that are bound to the affinity resin by passing a solution comprising a second chemical compound over the column; and (d) identifying proteins in the eluate, optionally obtaining a proteomic profile for the second chemical compound and, optionally, comparing the identified proteins (e.g., the proteomic profile) to proteins eluted from the column by a solution comprising the first chemical compound (e.g., to the proteomic profile for the first chemical compound).

### Embodiment 2

The method of embodiment 1, wherein: (1) the second chemical compound is a derivative or analog of the first chemical compound; or (2) the first chemical compound and the second chemical compound are selected from Tables 6-9.

### Embodiment 3

The method of embodiment 1 or 2, wherein identifying the proteins in the eluates comprises performing sodium dodecyl sulfate (SDS) polyacrylamide gel electrophoresis (PAGE).

### Embodiment 4

The method of embodiment 3, further comprising measuring intensities of bands in the gel by electronically scanning the gels and performing densitometry analysis.

### Embodiment 5

The method of any of embodiments 1-4, wherein proteins in the eluate are identified by performing tandem mass spectrometry (MS) analysis.

### Embodiment 6

The method of any of embodiment 5, further comprising excising separate bands from the gels and performing tandem MS analysis each excised band.

### Embodiment 7

The method of any of embodiments 1-6, wherein the first chemical compound is DMP543 or an analog or derivative thereof that inhibits KCNQ (Kv7) channel activity.

### Embodiment 8

The method of embodiment 7, wherein DMP543 is conjugated or covalently attached to the resin as follows:

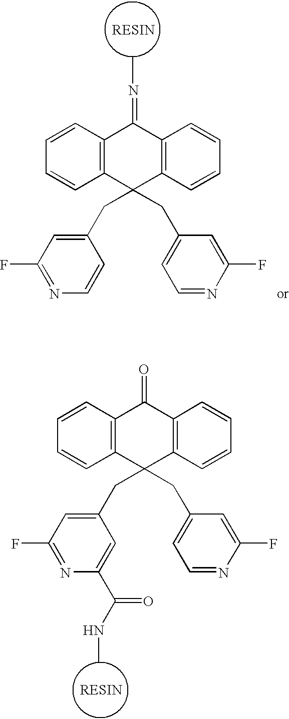

### Embodiment 9

The method of any of embodiments 1-6, wherein the first chemical compound is XE991 or an analog or derivative thereof that inhibits KCNQ (Kv7) channel activity.

### Embodiment 10

The method of embodiment 9, wherein XE991 is conjugated or covalently attached to the resin as follows:

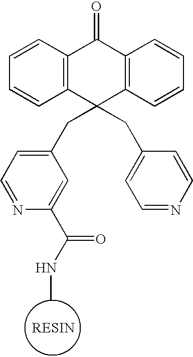

### Embodiment 11

The method of any of embodiments 1-6, wherein the first chemical compound is linopirdine or an analog or derivative thereof that inhibits KCNQ (Kv7) channel activity.

### Embodiment 12

The method of embodiment 11, wherein linopirdine is conjugated or covalently attached to the resin as follows:

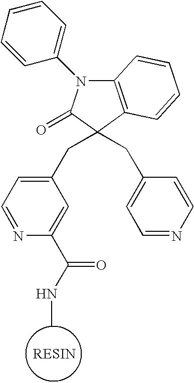

### Embodiment 13

The method of any of embodiments 1-12, wherein the biological sample is obtained from neural tissue, liver tissue, or heart tissue.

### Embodiment 14

A method comprising: (a) passing a biological sample comprising proteins over columns comprising a chemical-resin library, wherein each column comprises a separate member of the chemical-resin library; (b) washing each column to remove any non-bound proteins; (c) eluting any bound proteins from each column; and (d) identifying proteins in the eluates, optionally generating a proteomic profile for each column and optionally further comparing the proteomic profiles of two or more columns.

### Embodiment 15

A method comprising: (a) passing a biological sample comprising a target protein and optionally a non-target protein over a first column, the first column comprising an affinity resin for the target protein, the affinity resin comprising a resin conjugated or covalently attached to a first chemical compound that binds to the target protein; (b) washing the first column and removing proteins that are not bound to the affinity resin; (c) eluting proteins from the first column that are bound to the affinity resin; (d) identifying proteins in the eluate including the target protein and the non-target protein; (e) passing the biological sample comprising the target protein and optionally a non-target protein over a second column, the second column comprising an affinity resin for the target protein, the affinity resin comprising a resin conjugated or covalently attached to a second chemical compound that binds to the target protein; (f) washing the second column and removing proteins that are not bound to the affinity resin; (g) eluting proteins from the second column that are bound to the affinity resin; and (h) identifying proteins in the eluate including the target protein and optionally the non-target protein; wherein optionally the second chemical compound binds the target protein with a higher affinity than the first chemical compound and optionally the second chemical compound binds the non-target protein with a lower affinity than the first chemical compound.

### Embodiment 16

The method of embodiment 15, wherein: (1) the second chemical compound is a derivative of the first chemical compound; or (2) the first chemical compound and the second chemical compound are selected from Tables 6-9.

### Embodiment 17

The method of embodiment 15 or 16, wherein eluting of the first column is performed by washing the column with a solution comprising the first chemical compound and eluting of the second column is performed by washing the column with a solution comprising the second chemical compound.

### Embodiment 18

The method of any of embodiments 15-17, wherein identifying the proteins in the eluates of the first column and the second column comprises performing sodium dodecyl sulfate (SDS) polyacrylamide gel electrophoresis (PAGE).

### Embodiment 19

The method of embodiment 18, further comprising measuring intensities of bands in the gels corresponding to the target protein and optionally the non-target protein by electronically scanning the gels and performing densitometry analysis.

### Embodiment 20

The method of any of embodiments 15-19, wherein proteins in the eluate of the first column are identified by performing tandem mass spectrometry (MS) analysis.

### Embodiment 21

The method of any of embodiments 15-20, wherein proteins in the eluate of the second column are identified by performing tandem mass spectrometry (MS) analysis.

### Embodiment 22

The method of embodiment 18, further comprising excising separate bands from the gels and performing tandem MS analysis each excised band.

### Embodiment 23

The method of embodiment 18, further comprising excising separate bands from the gel comprising the eluate of the first column and performing tandem MS analysis on each excised band, thereby identifying the proteins in the eluate of the first column.

### Embodiment 24

The method of embodiment 19, wherein the affinities of the first chemical compound and the second chemical compound for the target protein and the non-target protein are determined by measuring intensities of bands in the gels corresponding to the target protein and the non-target protein.

### Embodiment 25

The method of embodiment 24, wherein: (1) the intensity of the band corresponding to the target protein in the eluate from the second column is no less than the intensity of the band corresponding to the target protein in the eluate from the first column; and (2) the intensity of the band corresponding to the non-target protein in the eluate from the second column is less than the intensity of the band corresponding to the non-target protein in the eluate from the first column.

### Embodiment 26

The method of any of embodiments 15-25, wherein the first chemical compound is DMP543 or an analog or derivative thereof that inhibits KCNQ (Kv7) channel activity.

### Embodiment 27

The method of claim 26, wherein DMP543 is conjugated or covalently attached to the resin as follows:

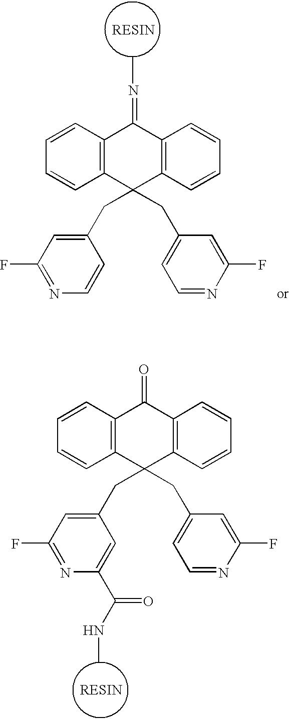

### Embodiment 28

The method of any of embodiments 15-25, wherein the first chemical compound is XE991 or an analog or derivative thereof that inhibits KCNQ (Kv7) channel activity.

### Embodiment 29

The method of claim 28, wherein XE991 is conjugated or covalently attached to the resin as follows:

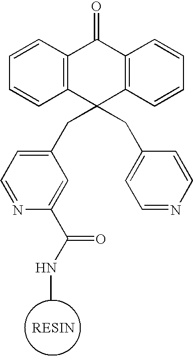

### Embodiment 30

The method of any of embodiments 15-25, wherein the first chemical compound is linopirdine or an analog or derivative thereof that inhibits KCNQ (Kv7) channel activity.

### Embodiment 31

The method of embodiment 30, wherein linopirdine is conjugated or covalently attached to the resin as follows:

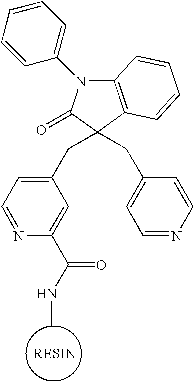

### Embodiment 32

The method of any of embodiments 15-31, wherein the biological sample is obtained from neural tissue.

### Embodiment 33

The method of any of embodiments 15-32, wherein the method is performed in order to obtain a chemical compound that binds to the target protein with an affinity no less than the affinity of the first chemical compound and that binds to the non-target protein with an affinity less than the affinity of the first chemical compound.

### Embodiment 34

The method of any of embodiments 15-33, wherein the target protein is a KCNQ (Kv7) channel protein.

### Embodiment 35

A method comprising: (a) passing biological sample comprising a KCNQ (Kv7) channel protein over a first column, the first column comprising an affinity resin for the target protein, the affinity resin comprising a resin conjugated or covalently attached to DMP543; (b) washing the first column and removing proteins that are not bound to the affinity resin; (c) eluting proteins from the first column that are bound to the affinity resin; (d) identifying proteins in the eluate including the KCNQ (Kv7) channel protein; (e) passing the biological sample comprising the KCNQ (Kv7) channel protein over a second column, the second column comprising an affinity resin for the target protein, the affinity resin comprising a resin conjugated or covalently attached to a derivative or analog of DMP543 that binds to the KCNQ (Kv7) channel protein; (f) washing the second column and removing proteins that are not bound to the affinity resin; (g) eluting proteins from the second column that are bound to the affinity resin; and (h) identifying proteins in the eluate including the KCNQ (Kv7) channel protein.

### Embodiment 36

A method comprising: (a) passing a biological sample comprising a target protein and a non-target protein over a first column over a first column, the first column comprising an affinity resin for the target protein, the affinity resin comprising a resin conjugated or covalently attached to a first chemical compound that binds to the target protein; (b) washing the first column and removing proteins that are not bound to the affinity resin; (c) eluting proteins from the first column that are bound to the affinity resin; (d) identifying proteins in the eluate including the target protein and optionally the non-target protein, thereby generating a proteomic profile for the first chemical compound; (e) passing the biological sample comprising the target protein and the non-target protein over a second column, the second column comprising an affinity resin for the target protein, the affinity resin comprising a resin conjugated or covalently attached to a second chemical compound that binds to the target protein; (f) washing the second column and removing proteins that are not bound to the affinity resin; (g) eluting proteins from the second column that are bound to the affinity resin; and (h) identifying proteins in the eluate including the target protein and optionally the non-target protein, thereby generating a proteomic profile for the second chemical compound; and (i) comparing the proteomic profile of the first chemical compound and the proteomic profile of the second chemical compound.

### Embodiment 37

A kit comprising one or more compounds of Tables 6-8 separately attached to a resin

### Embodiment 38

A library of chemical-resins where the chemical compounds of the chemical-resins are selected from Tables 6-8.

### EXAMPLES

The following examples are illustrative and not intended to limit the claimed subject matter.

### Example 1

### Chemical Proteomics-Based Drug Design: Target and Anti-Target Fishing with a Catechol-Rhodanine Privileged Scaffold for NAD(P)(H) Binding Proteins

Reference is made to Ge et al., J. Med. Chem. 2008; (15):4571-80, Epub Jul. 11, 2008; the content of which is incorporated by reference herein in its entirety.

Abstract

Drugs typically exert their desired and undesired biological effects by virtue of binding interactions with protein target(s) and antitarget(s), respectively. Strategies are therefore needed to efficiently manipulate and monitor cross-target binding profiles (ex. imatinib and isoniazid) as an integrated part of the drug design process. Herein we present such a strategy, which reverses the target=>lead rational drug design paradigm. Enabling this approach is a catechol-rhodanine privileged scaffold for dehydrogenases, which is easily tuned for affinity and specificity towards desired targets. This scaffold crosses bacterial (E. coli) cell walls, and proteome-wide studies demonstrate it does indeed bind to and identify NAD(P)(H)-binding proteins that are potential drug targets in Mycobacterium tuberculosis and antitargets (or targets) in human liver. This approach to drug discovery addresses key difficulties earlier in the process by only pursuing targets for which a chemical lead and optimization strategy are available, to permit rapid tuning of target/antitarget binding profiles.

Introduction

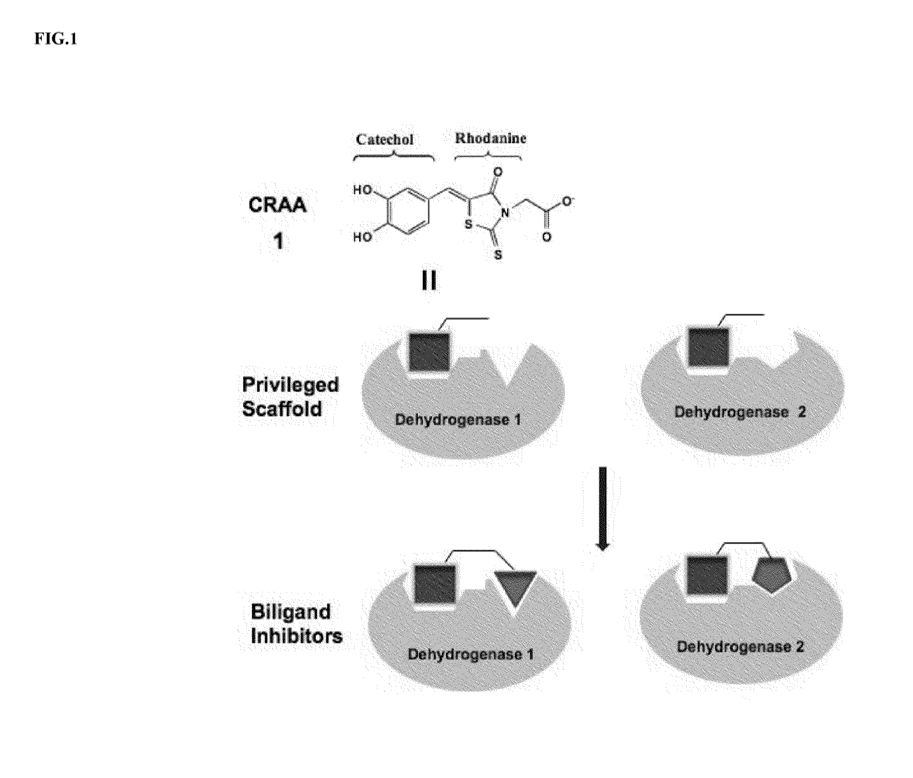

The drug discovery process is costly and often inefficient¹. Genomics and proteomics advances have presented the promise of improving efficiency, but this has largely translated into the identification of new drug targets, not new drugs. What is needed is a better coupling of the chemistry of drug design to advances in genomics and proteomics. To partially address this, chemical genetic approaches have been developed^{2, 3}, where enzyme inhibitors are used to knock out protein function. One advantage of chemical genetics over traditional genetics is that besides providing phenotypic data in the context of a whole organism, it yields an inhibitor for subsequent optimization in the drug discovery process. Still, this process is problematic in two ways: (a) one cannot be certain that the inhibitor binds only to the intended target, and (b) it is highly inefficient because new inhibitors must be designed for each new target of interest. The first question is relevant because binding to other proteins (antitargets) can lead to toxic side-effects. Further complicating matters, in other cases, such as imatinib⁴⁻⁶ (4-[(4-Methyl-1-piperazinyl)methyl]-N-[4-methyl-3-[([4-(3-pyridinyl)-2-pyrimidinyl]amino]-phenyl]benzamide methanesulfonate) and isoniazid⁷, off-target binding is actually thought to contribute to drug efficacy, thereby calling into question the one-target/one-drug dogma that serves as the foundation for rational drug design. The second question is relevant because the process of designing potent inhibitors that are acceptable drug leads can take years, and varies in difficulty from protein to protein, being nearly impossible for some protein targets—leading to the notion of “druggable” protein targets^{8, 9}. There is a vital need to identify “druggable targets” (those for which potent and selective inhibitors can be designed) as early in the drug discovery process as possible. To address this second concern, compounds can be designed based on “privileged scaffolds”¹⁰⁻¹³, which are drug-like^{14, 15} molecular structures that provide baseline affinity for a whole protein family. These scaffolds then serve as starting points for optimization against specific protein targets of interest in the family, usually by building a focused combinatorial library off of the scaffold. To this end, privileged scaffolds have been reported for kinases¹⁶, proteases^{17, 18} and GPCRs^{19, 20}. We have recently reported the first privileged scaffold for NAD(P)(H)-binding proteins²¹, based on a catechol-rhodanine ring system. Proteins in this family include the oxidoreductases (aka dehydrogenases), with drug targets such as HMG-CoA reductase (statin drugs), steroid-5α-reductase (finasteride), aldose reductase (diabetes), and a large number of infectious disease targets^{22, 23}, including enoyl CoA reductase, deoxyxylulose-5-phosphate reductoisomerase (DOXPR), and dihydrodipicolinate reductase (DHPR); this family even includes enzymes other than oxidoreductases, such as sirtuins, ADP-ribosylating enzymes and ligases. The catechol-rhodanine privileged scaffold has served as a template for building bi-ligand libraries, where the ligand attached to the scaffold is situated in the substrate pocket, thereby giving specificity to a particular enzyme in the family (FIG. 1). It has been used to generate multiple potent (K_{d}≦200 nM) and selective inhibitors for dehydrogenases, including DHPR and DOXPR²¹, with affinity and selectivity readily tuned by varying the fragment attached to the scaffold.

Despite the power of this scaffold, it has never been properly verified as being specific for NAD(P)(H)-binding enzymes in a proteome-wide manner. This is because a strategy was not available to assess cross reactivity (off-target binding) with other family members, in the context of a whole proteome—whether for the catechol-rhodanine scaffold itself, or for bi-ligand drug leads constructed from it, for specific targets. This gets to the first concern mentioned above. Recent advances in chemical proteomics^{6, 24, 25} now permit proteome-wide binding studies of the scaffold (and later, of bi-ligands), by covalently attaching scaffold to a resin, binding all protein family members in a proteome sample, then eluting with free scaffold (or bi-ligand), and identifying proteins with tandem MS. Such a strategy was recently used to assess binding profiles for currently-prescribed drugs, such as imatinib⁴⁻⁶ and isoniazid⁷. Both of these drugs were thought to bind tightly to a single target, and it was later discovered that their biological efficacy might actually be due to binding to multiple targets. The strategy and tools presented in this paper would now permit the assessment and optimization of cross-target binding profiles (target/antitarget) across a proteome as an integral part of the drug design process; in this manner, binding profiles could be correlated with biological efficacy upfront in a rational manner, rather than relying on serendipitous and unbeknownst off-target effects.

The strategy proposed herein depends crucially on the availability of a privileged scaffold that binds to a protein family (dehydrogenases, in this case) and that has been designed in such a way that it can be quickly modified to produce potent inhibitors for a given family member (building bi-ligands, in this case). The latter has already been verified²¹ for the privileged scaffold that is the topic of this paper, which is based on the catechol rhodanine acetic acid 1 (CRAA) shown if FIG. 1. But, is this scaffold a viable starting point for drug discovery? While the thiazolidine ring (rhodanine is a type of thiazolidine) has been reported by Poupaert et al.²⁶ as a frequently occurring heterocyclic motif in anti-inflammatory, antipsychotic and anticonvulsant drugs, the rhodanine ring is less common. But, it does occur in drugs such as Epalrestat ((2-[(5Z)-5-[(E)-3-cyclohexyl-2-methylprop-2-enylidene]-4-oxo-2-thioxo-3-thiazolidinyl]acetic acid)), a potent inhibitor of aldol reductase (AR), and has been shown to have no significant toxicity in recent clinical trials^{27, 28}. The catechol group, though present in a number of plant-derived natural products, can have toxicity in some cases when it is oxidized to an o-quinone, which can then alkylate cellular macromolecules or generate reactive oxygen species^{29, 30}. As such, 1 does seem to be a viable scaffold upon which to build drug leads, using the strategies described herein, with the caveat that the o-catechol may need to be replaced if there is any toxicity.

The chemical proteomic strategy proposed herein also relies on attaching a dehydrogenase-specific ligand to a resin, and using that affinity column with subsequent digestion of the eluted proteins and subjecting the tryptic peptides to electrospray LC/MS followed be searching the MS/MS data against an appropriate subset of the Uniprot database to identify all (reasonably abundant) proteins in a proteome that bind the ligand. While affinity purification using native cofactor has been applied to dehydrogenases for over 30 years³¹⁻³³, it has never been coupled to tandem MS to probe binding profiles for a dehydrogenase-targeted privileged scaffold. And more broadly, although there is emerging interest in using affinity chromatography coupled mass spectrometry to probe protein-ligand interactions across a proteome³⁴, there is a need for more efficient coupling of this assay methodology earlier in the drug design process, using the chemical leverage provided by privileged scaffolds to create an integrated drug discovery approach that blends: (a) a broad assessment of target/antitarget binding profiles, (b) a pragmatic selection of druggable targets, and (c) an efficient chemical strategy for tuning target/antitarget affinity. This paper presents a foundation for such a strategy, applied using the first such privileged scaffold for NAD(P)(H) binding proteins.

Results

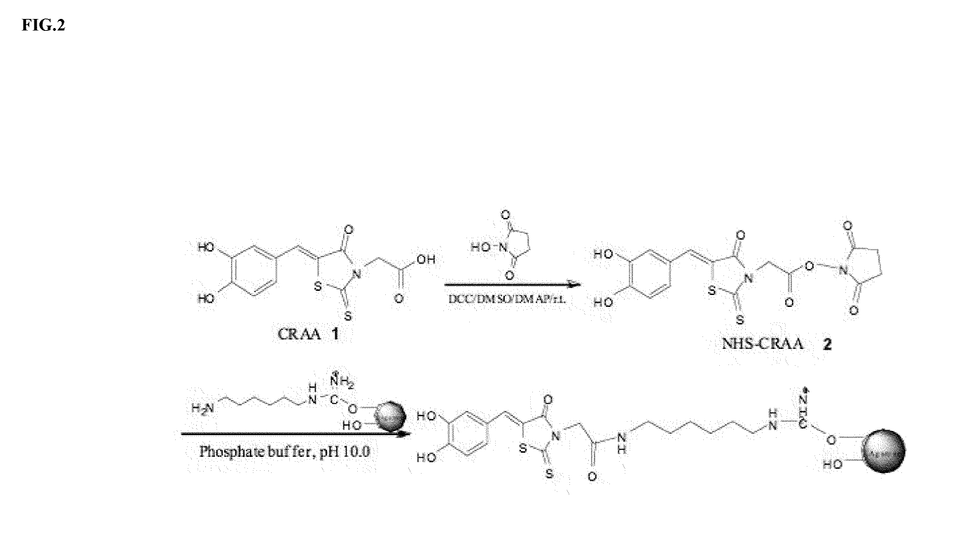

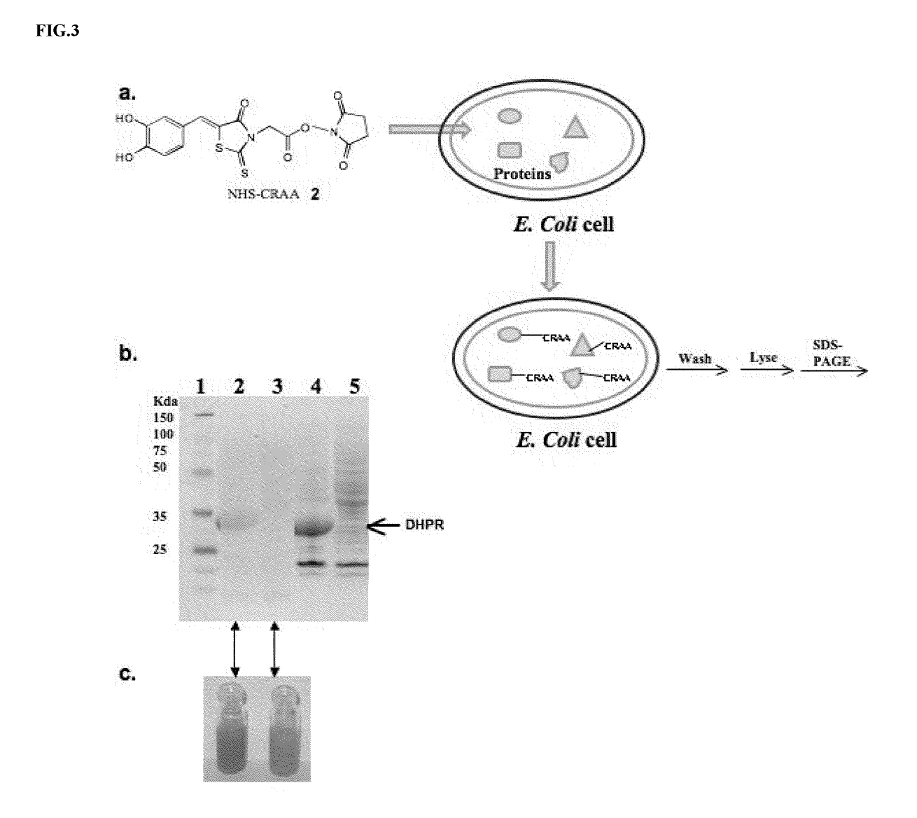

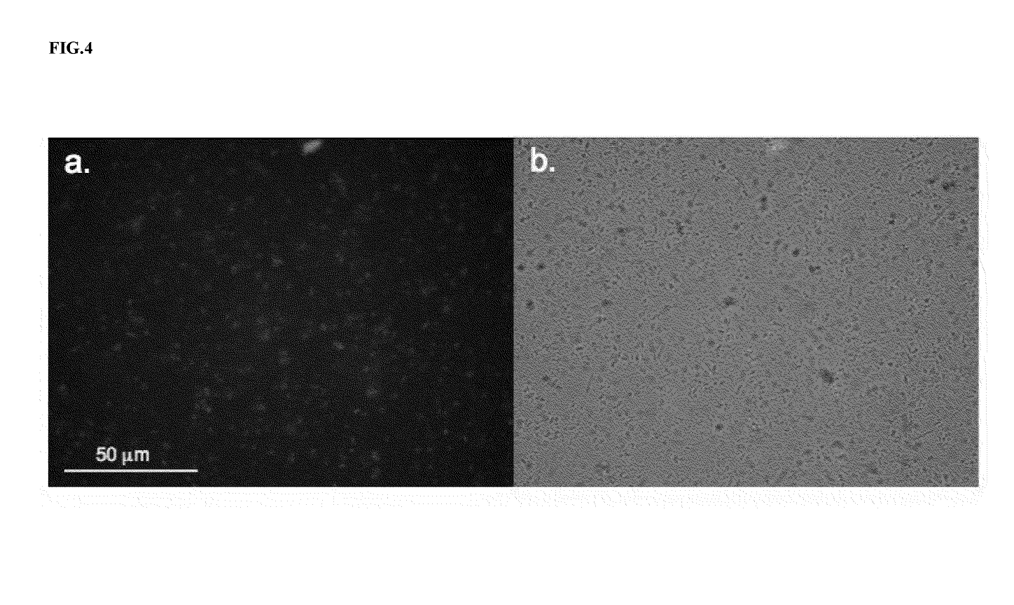

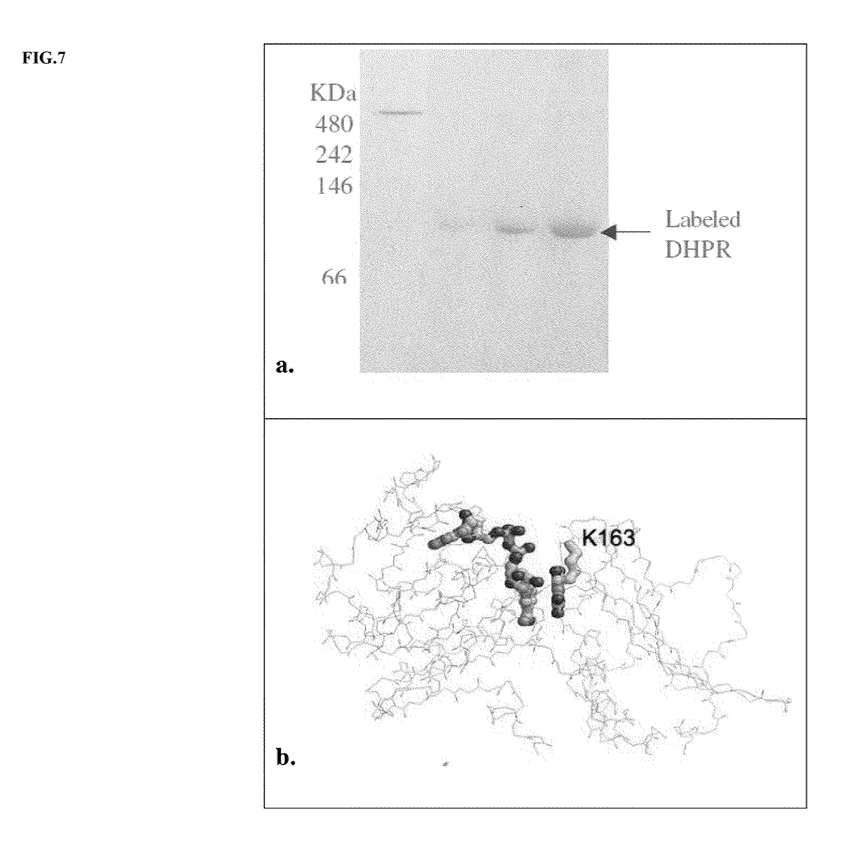

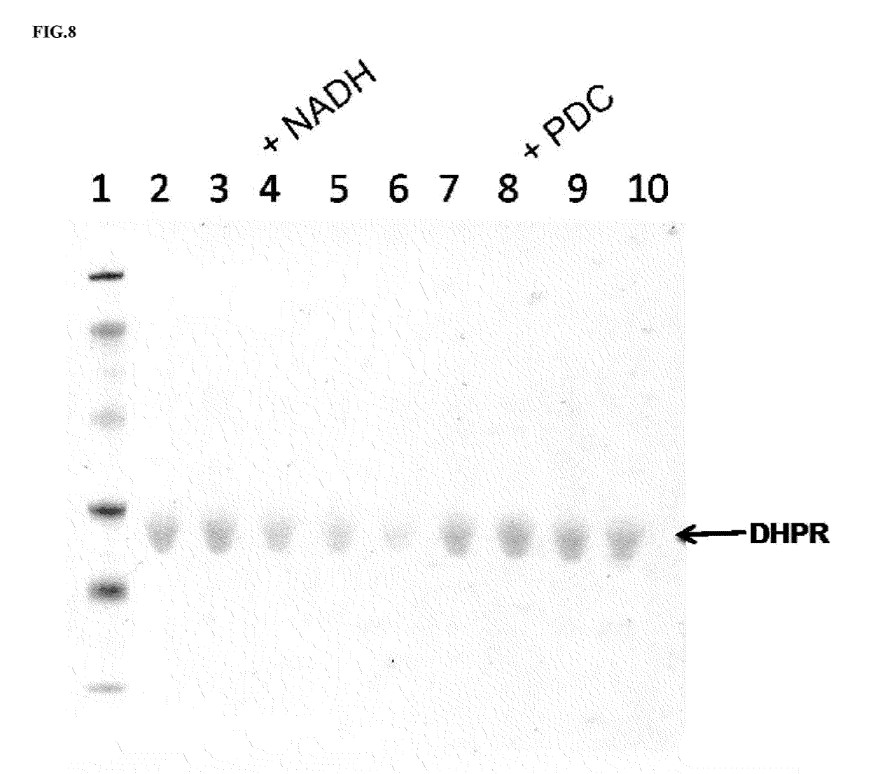

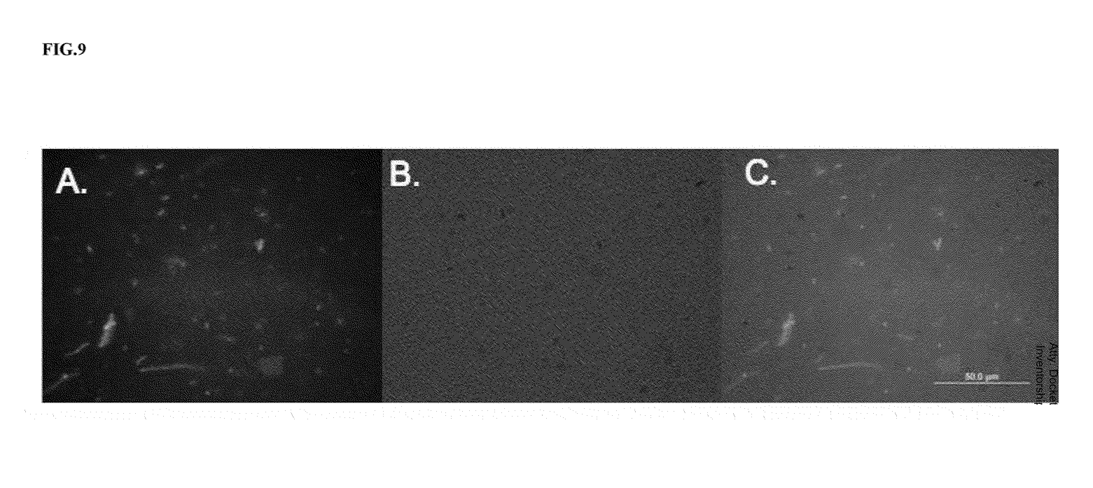

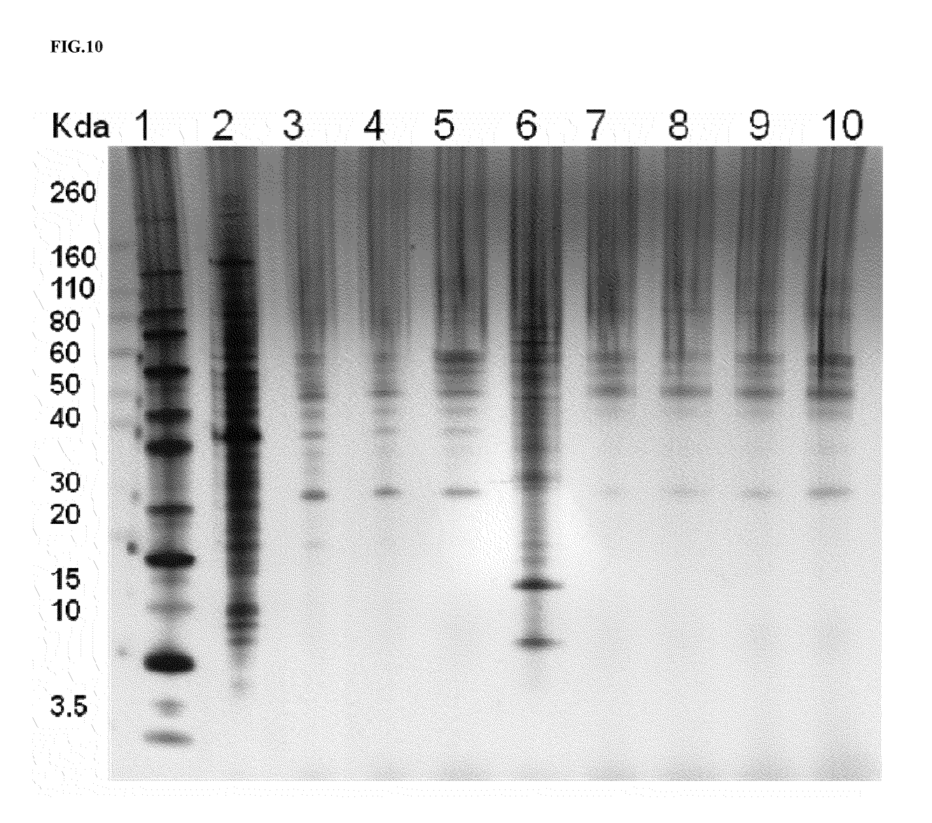

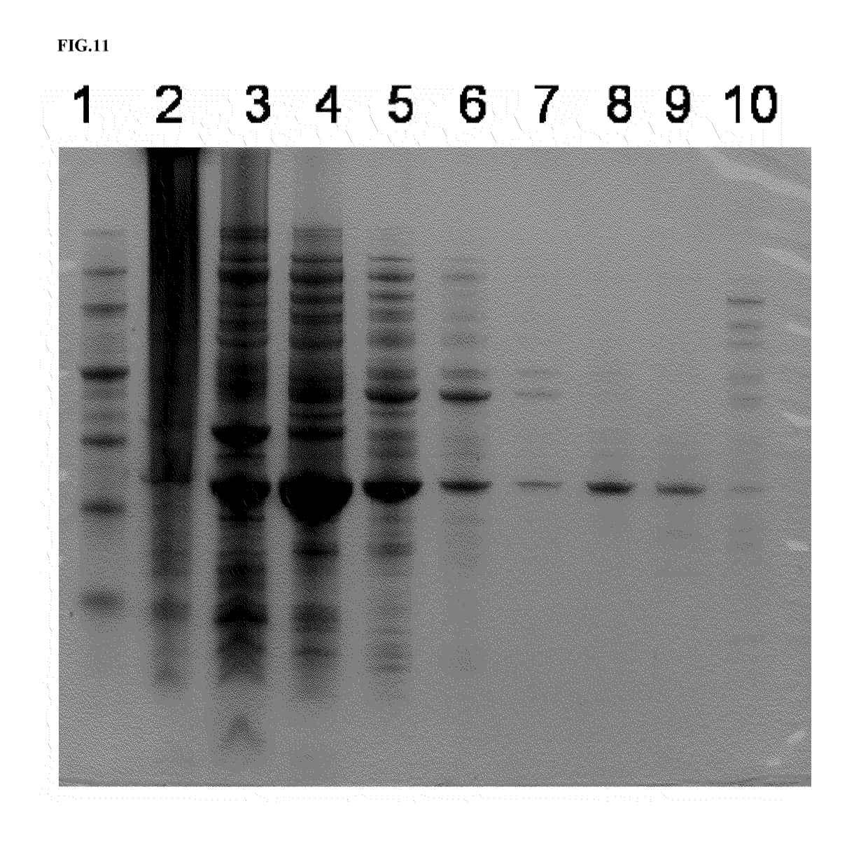

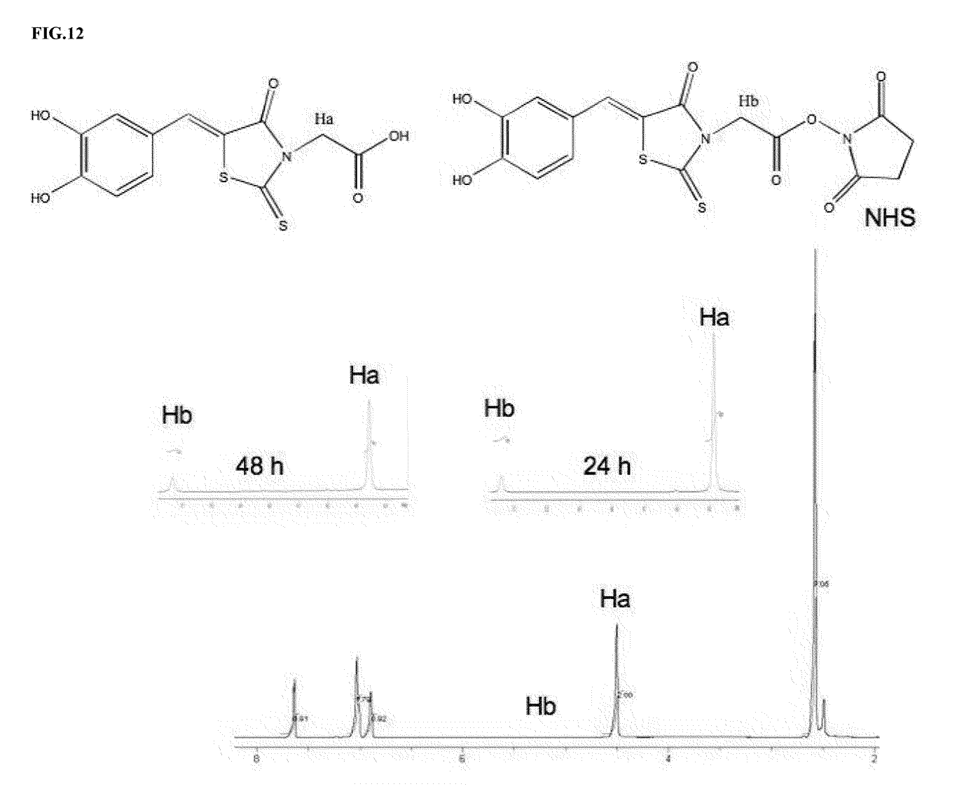

2 (NHS-CRAA) uptake into E. coli cells and labeling of DHPR. To assess whether 1 (CRAA) can make it across bacterial cell walls, and therefore whether 1 is a viable scaffold for anti-infective drug design efforts, experiments were performed to determine if its N-hydroxysuccinimide ester, 2 (NHS-CRAA; FIG. 2), could enter E. coli and label intracellular DHPR (dihydrodipicolinate reductase). DHPR is an anti-infective drug target, and is known to bind the CRAA scaffold (1)²¹. The NHS-CRAA active ester (2)³⁵ was synthesized as in FIG. 2. The NHS (N-hydroxysuccinimide) group reacts with amines, and since it is attached to the linker position of 1 (where the acetic acid chain is attached), it should reside at the interface of the NADH and substrate binding sites²¹ (FIG. 1), near lysine 163³⁶ (see FIG. 7). Indeed, DHPR is labeled with the NHS-CRAA (2) active ester, based on imaging of an SDS-PAGE gel of labeled protein (FIG. 8). Increasing NADH concentration appears to decrease band intensity while PDC has little effect up to 9 mM. Labeling is partially blocked by NADH (FIG. 8), indicating the NHS-CRAA (2) probe is in fact binding and labeling (at least partially) in the active site of DHPR. Next, to explore whether cell wall penetration occurs, uptake of 2 was measured into E. coli that was expressing E. coli DHPR. Since we have recently shown that the CRAA scaffold (1) is bifunctional, in that it is itself weakly fluorescent³³, protein labeling could be monitored by fluorescence imaging of SDS-PAGE gels of crude cell extracts. In-cell studies were performed by overexpressing DHPR in E. coli, then exposing intact cells to the NHS-CRAA probe (2), washing and lysing cells, and then running an SDS-PAGE gel to see if the probe was able to covalently label the DHPR (FIG. 3a). Any NHS-CRAA probe (2) that was nonspecifically associated with the cells was quenched by treatment with 100 mM Tris before lysis. Gels show that significant labeling of the DHPR did occur within the intact E. coli cells (FIG. 3b), indicating that scaffold can cross the cell wall in order to gain access to DHPR. That there is more labeling in cells expressing DHPR is evident based on the more intense color for cells expressing DHPR (FIG. 3c) and on fluorescence images of the cells (FIG. 3d).

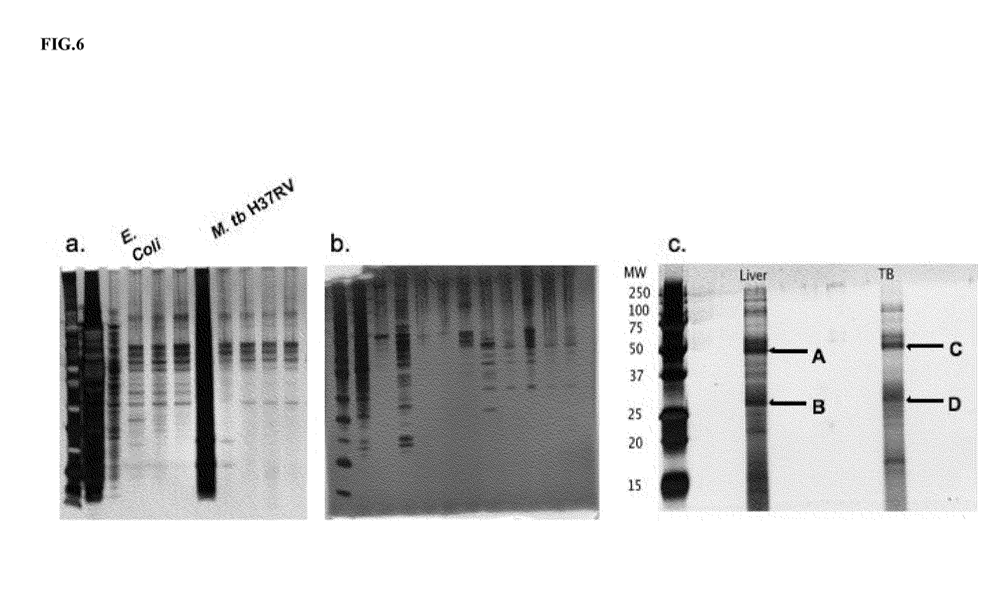

CRAA (1) affinity chromatography and nanospray-LC/MS/MS. Our proteome fishing studies (FIG. 5) require, a resin with a privileged scaffold, in this case 1, covalently attached. The NHS-CRAA (2) active ester (FIG. 2) was used to prepare this affinity resin, to permit purification of dehydrogenase (NAD(P)(H)-binding protein) subproteomes from protein mixtures. Crude cell lysates from E. coli and M. tuberculosis were both loaded onto the affinity column, and proteins eluted using free CRAA (1) probe, as shown in FIG. 5. SDS-PAGE analysis of both microbial samples showed very similar patterns (FIG. 6a), although analysis of proteins that were identified from M. tuberculosis (vide infra, Table 2) indicate that some proteins have no E. coli homologs, and for those that do, masses differ—so, the apparent similarity in gel patterns is more likely due to the prevalence of proteins in these molecular weight ranges in both microbes. As with the microbial samples, human liver proteins were loaded onto the affinity resin and eluted with 1 (FIG. 6b), to determine the CRAA-binding profile for the liver proteome. It is noteworthy in this gel, which shows wash and elution fractions, that proteins are specifically eluted by free 1.

In FIG. 6, the collection of bands that are observed define what is called the proteome profile for CRAA, and is comprised of both target and non-target proteins. Other chemical fragments can be covalently attached to the CRAA scaffold to create derivatives, and these may be used in place of CRAA to elute proteins from the CRAA-affinity column. Then proteomic profiles for these derivates can be generated. Derivatives having a greater intensity of target bands relative to non-target bands are identified. In the case of an anti-infective drug, one is also concerned with avoiding binding to proteins in human organs such as liver (these would be considered non-target proteins), as the intention is to bind specifically to proteins in M. tuberculosis and not human. Binding to human proteins can lead to toxic side effects, and particular organs of concern for toxic side effects include liver, kidney and heart.

In FIG. 5, the bottom branch illustrates how chemical additions (illustrated by the triangle) are made to the scaffold (illustrated by the square), to tune the specificity of the drug lead molecule so only desired target proteins are eluted by the modified scaffold or drug. Illustrative examples of such chemical modifications to the scaffold are provided in Sem et al. (2004) Chem. Biol. 11, 185, the contents of which are incorporated herein by reference in their entirety. Other suitable drug scaffolds are provided in Table 9 and can be modified in this manner. For example, chemical additions could be made to the phenolic group of the glitazone scaffold that is common to both Actos and Avandia. In this case, glitazole is represented by the square in the figure, and this is tethered easily to other chemical fragment by nucleophilic attack of the phenolate on an appropriate electrophile (e.g., via a Williamson ether synthesis).

For both human liver and M. tuberculosis eluents, nanospray-LC/MS/MS analysis was performed followed by searching the MS/MS data against an appropriate subset of the Uniprot database to determine which CRAA-binding proteins were present in reasonably high abundance. To complement this whole proteome (actually subproteome) analysis, CRAA-eluted fractions were also separated using SDS PAGE, and protein bands at ˜35 kDa and ˜55 kDa (FIG. 6c) were in-gel digested, then peptides were extracted from the gel and analyzed as above. In both cases, proteins were first digested with trypsin, then zip-tip cleaned and injected into an LC-MS system (LTQ with a linear ion trap from Thermo-Fisher). Whole subproteome analyses are in Tables 1 (human liver) and 2 (M. tuberculosis), while analysis of extracted bands is in Table 3. Complete data sets, even for very low scoring hits, are given in Tables 4 and 5. Generally, LC/MS data indicate that >50% of these proteins are dehydrogenases or other NAD(P)(H) binding proteins, as expected. Better identifications were obtained from the human liver sample, perhaps because the M. tuberculosis sample workup involves irradiation, which may cause some protein damage. In any case, several M. tuberculosis proteins could be identified with high certainty. Analysis of extracted bands was intended as a check on the whole subproteome analyses, although in general there was lower signal-to-noise (and, as a consequence, scores) for these samples. Still, there is generally good agreement between extracted band data and whole subproteome analysis, especially when scores are higher (>10) and percent coverage of the protein sequence is more complete (≧7%).

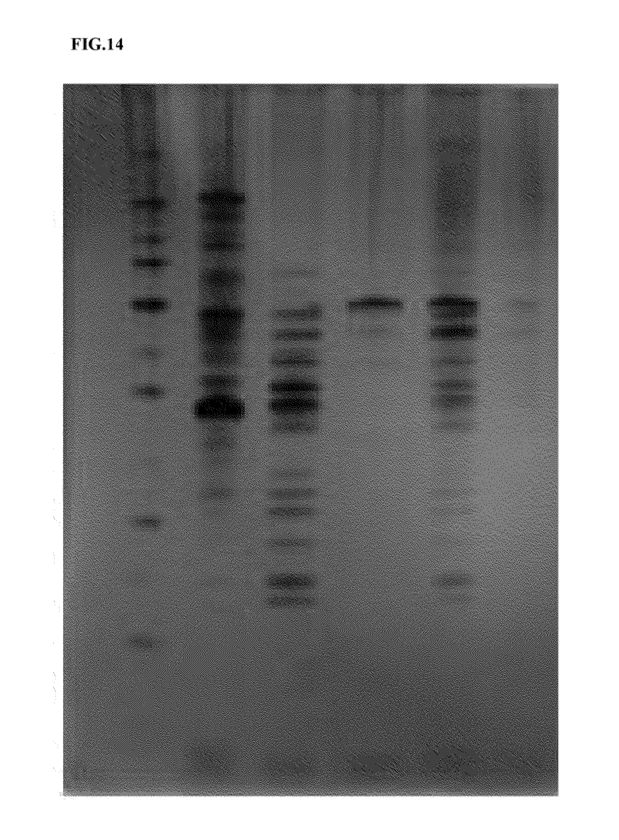

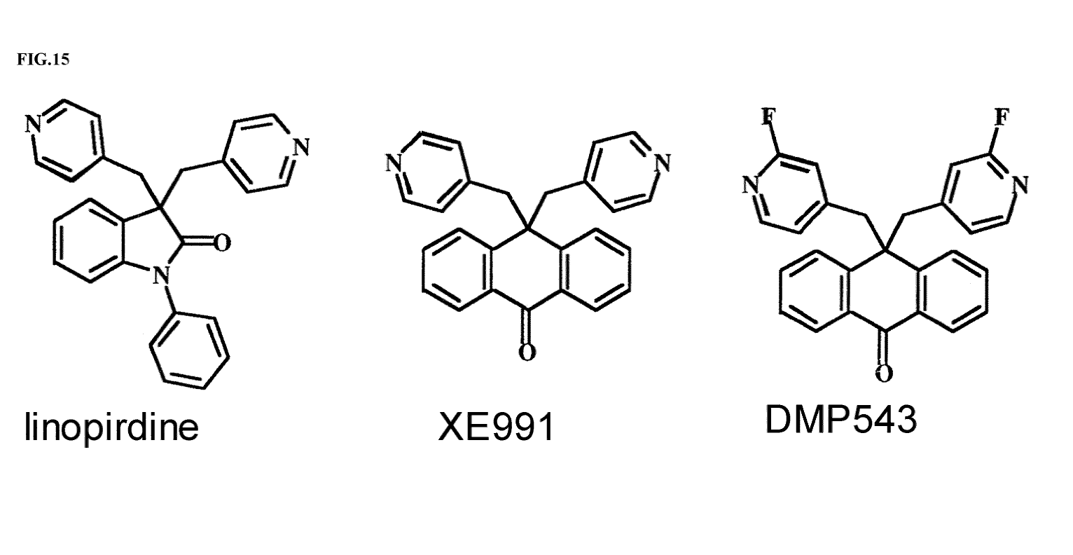

Of the highest scoring human liver proteins (Table 1), 5 out of 6 (excluding keratin, a very abundant protein) were dehydrogenases. The top hit, malate dehydrogenase, has more than 50% peptide coverage and a very high score, while glutamate, aldehyde and retinal dehydrogenases also had high percent coverage (>20%). Binding of 1 to two of these dehydrogenases (glutamate and malate) was subsequently verified experimentally in NMR STD (saturation transfer difference) binding assays (FIG. 13). Other dehydrogenases that appear to bind 1, based on lower but still statistically significant scores and percent coverage, include (FIG. 14): alcohol dehydrogenases, isocitrate dehydrogenase, alpha-aminoadipic semialdehyde dehydrogenase (gi116241244), and NADP-dependent leukotriene B4 12-hydroxydehydrogenase (gi23503081). It should be noted that for tandem MS analysis of bands at around 55 kDa and 35 kDa, isocitrate/aldehyde and malate dehydrogenases, respectively, are again identified with high certainty, confirming that they do indeed bind to 1.

As with the liver proteins, M. tuberculosis proteins were bound to the CRAA-affinity resin, then eluted with free CRAA (1) and fractions analyzed using electrospray LC/MS/MS. Of the highest scoring (score >13)M. tuberculosis proteins (Table 2), there were 4 possible pyridine nucleotide-binding proteins out of 6 proteins identified. The other proteins bind ATP, so the CRAA scaffold (1) may have some modest affinity for ATP binding sites as well. Interestingly, three of the proteins had no annotated function, but a subsequent NCBI search (i.e. updated annotation) and BLAST alignments identified the closest homologs to in fact be NAD(P)(H) binding proteins. This highlights the potential value of CRAA (1) target fishing in functional proteomic efforts, by even capturing uncharacterized proteins and providing suggestive data on their cofactor binding preferences, as well as the start of a chemical genetic probe.

Discussion

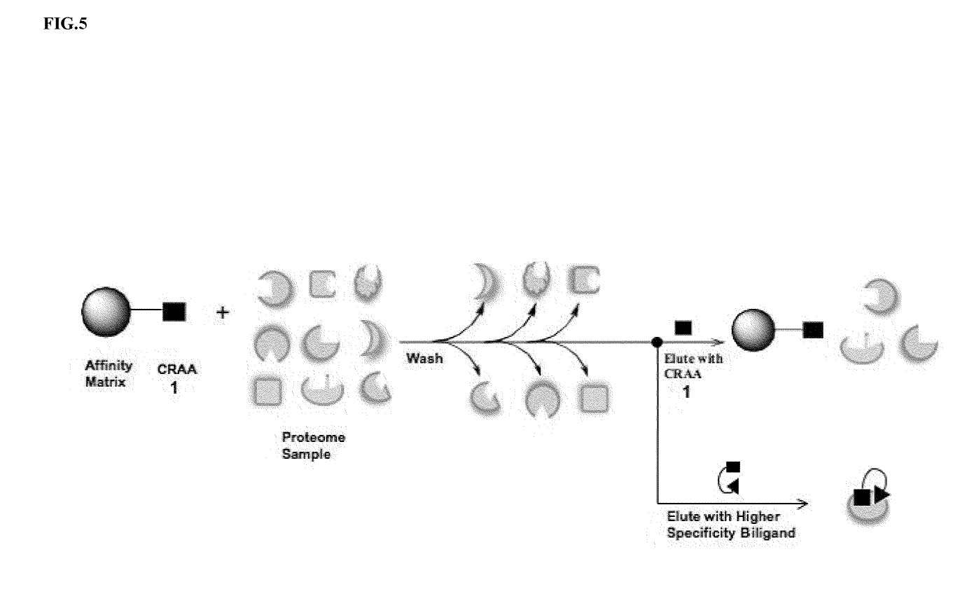

The methods presented herein were developed to address two of the major roadblocks in drug design projects, and in the development of chemical genetic probes (i.e. functional genomics): (a) there is a need to know the binding profile (target; antitarget binding) for a molecule as broadly as possible, whether it is a privileged scaffold that targets many proteins in a gene family, or a highly specific drug lead intended for one protein, and (b) there is a desperate need to speed up chemistry by including, integral to this process, a strategy for rapid tuning of a binding profile—this is accomplished by using a privileged scaffold that can be rationally modified to target a protein of interest (FIG. 1). A central element of this pragmatic approach to drug discovery is to make sure that protein targets are only pursued if a drug-like inhibitor is already in hand, which can be rationally modified for higher affinity with minimal effort. This addresses upfront, the common concern over whether a protein target is “druggable”. One additional drug discovery challenge, in the case of anti-infectives, is the formidable barrier of needing to cross the microbial cell wall. The studies presented herein present a strategy to drug discovery that attempts to address all of these problems, with a focus on NAD(P)(H)-binding proteins. The approach relies on the availability of a privileged scaffold that targets a gene family, and that is easily modified to achieve higher affinity for a given target. We have previously described such a probe for NAD(P)(H)-binding proteins²¹, which is shown in FIG. 1. The study presented herein extends this work by: (a) showing that this scaffold is able to cross bacterial cell walls (FIGS. 3, 4), (b) demonstrating that it truly is a privileged scaffold for NAD(P)(H) binding proteins (Tables 1-3) based on proteome-wide profiling (FIGS. 5, 6), and (c) identifying potential drug targets (Table 2) and antitargets (Table 1) to be pursued in future drug design efforts, coupled with proteome-wide profiling studies (FIG. 5).

With regard to penetrating bacterial cell walls, uptake studies were performed by monitored labeling of intracellular proteins using the CRAA (1) privileged scaffold tethered to an amine-reactive reagent. This is effectively an activity-based probe, analogous to those described for other protein families in the field of chemical proteomics^{38, 39}, but not yet reported for NAD(P)(H) binding proteins. The attachment point for the NHS group was chosen at the end of the CRAA (1) linker, in the position that is normally proximal to or in the substrate site (FIG. 1), so will only label proteins that have an amine in that position. Fortunately (and by design), DHPR has an amine in this position, so could be labeled. Incubation of purified DHPR with NHS-CRAA (2) does in fact lead to covalent labeling (FIGS. 7, 8). NHS-CRAA is expected to bind in the NADH pocket, with the NHS group proximal to lysine 163. Labeling is also observed if intact E. coli cells that are overexpressing DHPR are exposed to probe (FIGS. 3, 4). This could only happen if the probe can cross the cell wall, so provides unambiguous evidence that there is nothing about the CRAA scaffold (1) that would inherently preclude transport across cell walls. Of course, cell wall penetration will vary significantly depending on the bacteria in question, and based on what is attached to the CRAA (1) linker (as in FIG. 1), but these results are encouraging that at least in some cases cell wall penetration will be possible.

Next, to assess whether the CRAA scaffold (1) is a privileged scaffold for NAD(P)(H)-binding proteins, and to identify potential target and antitarget proteins, crude cell lysates from E. coli and M. tuberculosis were both loaded onto the affinity column, and proteins eluted using free CRAA (1) probe (FIG. 5). SDS-PAGE analysis of both microbial samples showed very similar patterns (FIG. 6a), suggesting some overlap in their dehydrogenase subproteomes and corresponding binding profiles for the CRAA privileged scaffold (1); although, some of this apparent overlap may also be due to the prevalence of proteins in this molecular weight range. Because Mycobacterium tuberculosis is of greater interest as a drug target^{40, 41}, proteomics studies were pursued to identify potential targets in its proteome. Interestingly, 4 out of the top 6 scoring proteins from the Mycobacterium tuberculosis proteome were NAD(P)(H) binding proteins, although this was not obvious based on the initial annotation of the database (3 out of the 4 hits were for proteins of undefined function). An analogous study with a human liver proteome sample also resulted in the identification of proteins that were mostly (>50%) NAD(P)(H)-binding proteins. Given that most proteomes are comprised of <5% dehydrogenases²³, the CRAA scaffold (1) appears to have good selectivity for this gene family. Now, any proteins that were identified in either the human or Mycobacterium tuberculosis proteomes have a baseline affinity for 1, so more potent inhibitors could easily be made for a target of interest using the bi-ligand design strategy outlined in FIG. 1, and previously validated²¹. Only pursuing protein targets for which the start of a potent inhibitor/drug lead is available is highly pragmatic, because it identifies “druggable” targets at the start of the drug discovery process. But, are any of the identified proteins in Tables 1 and 2 drug targets, and/or are they worth pursuing as targets of chemical genetic probes for basic research objectives (i.e. functional genomics)?

Any drug designed to be an anti-infective would need to be optimized so as to not disrupt function of vital proteins in the human proteome. And, since the liver is the body's first line of defense (after passage through the intestinal mucosa) before drugs go into the general circulation, proteome profiling was done against the human liver proteome. Of the human liver proteins identified (Table 1), 5 out of 6 (excluding keratin) were dehydrogenases. In terms of antitargets of concern, any drug leads designed using the CRAA (1) privileged scaffold (FIG. 1) should certainly be tested against malate, glutamate, isocitrate, and the various aldehyde dehydrogenases listed in Table 1. It should also be noted that some of these proteins may prove to be useful targets for human disease, in their own right. In this regard, it is interesting that CRAA (1) has affinity for various aldehyde dehydrogenases. This is perhaps not surprising, because the drug Epalrestat, also known as ONO-2235^{27,28}, also contains a rhodanine core, and is an aldol reductase inhibitor used to treat diabetes. Indeed, this suggests that our CRAA core (1) might be used as a starting point for building other aldose reductase inhibitors, with different and tunable off-target binding profiles. Another human enzyme that may bind 1 is NADP-dependent leukotriene B4 12-hydroxydehydrogenase, which is involved in eicosanoid inactivation, and is a target of indomethacin⁴² as well as other nonsteroidal anti-inflammatory drugs (NSAIDs)⁴³. Our proteome fishing data suggest that 1 might also be pursued as a starting point for inhibitors of this enzyme, by properly tuning affinity based on what fragments are added to the scaffold (FIG. 1). Another enzyme that may bind 1 is alpha-aminoadipic semialdehyde dehydrogenase (AASD). Genetic deficiency in AASD is known to cause pyridoxine-dependent epilepsy^{44, 45} While seizures in such individuals cannot be prevented using anti-epileptic drugs, they can be avoided by treatment with pyridoxine⁴⁶. So, it appears that AASD is an antitarget to be avoided. But, any potential problems from transient inhibition of AASD are likely to be less severe than the genetic knockout just described, and in any case could be alleviated by treatment with pyridoxine. Conversely, the CRAA scaffold (1) could be used as a starting point for designing a more potent inhibitor of AASD (FIG. 1), for chemical genetic studies in model organisms that contain close homologs of human AASD, such as zebrafish (gi27882244), rat (gi149064286), and xenopus (gi51703516).

If any human proteins are to be pursued as drug targets, specificity should be checked against the other metabolically important dehydrogenases listed in Table 1, to avoid toxicity and to achieve an acceptable therapeutic index. So, an important outcome of the human proteome data is: (a) a list of targets that could be pursued in subsequent drug discovery efforts, especially for diabetes (aldose reductase) and inflammation (NADP-dependent leukotriene B4 12-hydroxydehydrogenase), (b) a list of human antitargets for these drug discovery efforts, and (c) a proteomics-based strategy for assessing binding profiles (described in FIG. 5) to assess off-target effects. Finally, these data confirm that 1 is behaving as a privileged scaffold for dehydrogenases, in the context of the human liver proteome.

Towards the goal of using the CRAA privileged scaffold (1) in anti-infective drug discovery efforts, analogous proteome fishing studies were performed using crude cell lysates from Mycobacterium tuberculosis. As with the human liver proteins, M. tuberculosis proteins were bound to the CRAA-affinity resin, then eluted with free 1 and fractions analyzed using tandem MS. Of the highest scoring M. tuberculosis proteins (score >13; Table 2), there were 4 possible pyridine nucleotide-binding proteins out of 6 proteins identified. Interestingly, three of the captured proteins had no annotated function, but subsequent NCBI searches and BLAST alignments identified the closest homologs to be NAD(P)(H) binding proteins; this highlights the value of CRAA-based proteome fishing in functional proteomic efforts, even providing the start of a chemical genetic probe to later explore function. There is also some likelihood that one or more of these proteins could be drug targets. For example, the top scoring protein in Table 2 has high homology to a coenzyme F420-dependent N5,N10-methylene tetrahydromethanopterin reductase. Coenzyme F420 was first discovered in methanogenic archaea^{47, 48}, and is now known to be present in mycobacteria. Indeed, Daniels has suggested that targeting of F420-dependent enzymes might be pursued as a new strategy for killing mycobacteria⁴⁹. RibD, another Mycobacterium tuberculosis hit (Table 2), is essential for synthesis of riboflavin. While this may not be a viable drug target, a potent inhibitor of RibD would provide a chemical knockout to complement genetic knockouts of RibD (such mutants are riboflavin auxotrophs⁵⁰), to explore function. One potential application might be to create transient vitamin B2 auxotrophy, if one wanted to incorporate isotopically labeled riboflavin into a microbially expressed protein. Finally, the two “putative uncharacterized proteins” in Table 2 are also of interest, not just as potential drug targets, but because chemical genetic probes might help to better define their function. One of these proteins has highest homology to 17-β-hydroxysteroid dehydrogenase/Hydratase-dehydrogenase-epimerase; but, very little is known about the role of 17-β-keto dehydrogenases in microbes. The human homolog (17-β-hydroxysteroid dehydrogenase) is involved in the synthesis of estradiol from estrone, so is a target for breast cancer and endometriosis⁵¹. What metabolic role the microbial enzyme plays, and whether it is a viable drug target, is not known⁵², but could certainly be probed with chemical genetic probes based on the CRAA scaffold (1). The other uncharacterized protein identified in Table 2 is in the nitroreductase family. Purkayastha et al.⁵³ have noted that nitroreductases may play a role in helping mycobacteria respond to different host conditions; for example, a nitroreductase is upregulated when mycobacteria are inside the macrophage. Because mycobacteria survive and multiply inside macrophages⁵⁴, it is important to better understand the enzymes that are upregulated and perhaps facilitate their survival in this environment. Dissecting this regulatory cascade might uncover new drug targets, and could possibly provide a better basic understanding of how the bacteria can hide within the host's own defense system. Higher affinity ligands constructed off the CRAA scaffold (1) would minimally serve as chemical genetic probes, and perhaps even as drug leads.

Of the scaffold-binding M. tuberculosis proteins identified, it is certainly not yet known which (if any) will be useful drug targets, because of a lack of proper annotation. But, the above discussion points out an especially useful feature of the CRAA probe (1)—it can be used both as a platform for drug design, as well as for development of chemical genetic probes. That is, bi-ligands designed with specificity for these proteins of unknown function could then be used to explore phenotypic effects of a chemical knockout. If the phenotypic effect suggests a mechanism to kill the microbe, then at least there is the start a drug lead in hand. The intention of this study, then, is to prepare a foundation for future drug design and chemical genetic initiatives, by providing a chemical scaffold for optimization (CRAA, 1), a strategy for using it to generate new and potent bi-ligand inhibitors (FIG. 1), a proteome-wide method for assessing target and antitarget binding broadly (FIGS. 5 and 6), to correlate with phenotypic effects, and a list of targets and antitargets in both human and Mycobacterium tuberculosis to begin pursuing (Tables 1-3).

Methods

Equipment and materials. Nano-HPLC-mass spectrometry was performed using an LTQ mass spectrometer (Thermo-Fisher) coupled to a Surveyor HPLC system (Thermo Fisher) equipped with a Finnigan Micro AS autosampler. The instrument was interfaced with an Aquasil, C18 PicoFrit capillary column (75 μm×10 cm) from New Objective. A Kodak Image Station 2000MM System was used for gel fluorescence scanning (FIG. 3b), and an Olympus BX60 microscope for fluorescene imaging of cells (FIG. 4). All Novex gel products for the SDS-PAGE experiments were from Invitrogen, as was the SilverQuest staining kit. All salts, buffers, enzymes, and other chemical reagents are from Sigma-Aldrich and are of biochemical reagent grade, unless specified otherwise. The ω-aminohexyl-agarose and the human liver proteins (cytoplasmic) are also from Sigma. The M. tuberculosis H37Rv whole cell lysate was from Colorado State University. These proteins are from cells that were grown in glycerol-alanine stocks for 14 days, then washed with PBS. After gamma-irradiation (to inactivate) cells were disrupted (French Press) and the lysate centrifuged to remove cell debris. Lysis buffer was PBS with 8 mM EDTA and protease inhibitors.

Synthesis of 1 (CRAA) 5-[(3,4-dihydroxyphenyl)methylene]-4-oxo-2-thioxo-3-thiazolidineacetic acid. Synthesis was largely as described before³⁷. Briefly, 3-rhodanine acetic acid was reacted with 3,4-dihydroxybenzaldehyde in acetic acid/acetate at 90° C. for 6 hours. After cooling, yellow crystals were poured into cold water, filtered, washed, and then crystallized from acetic acid.

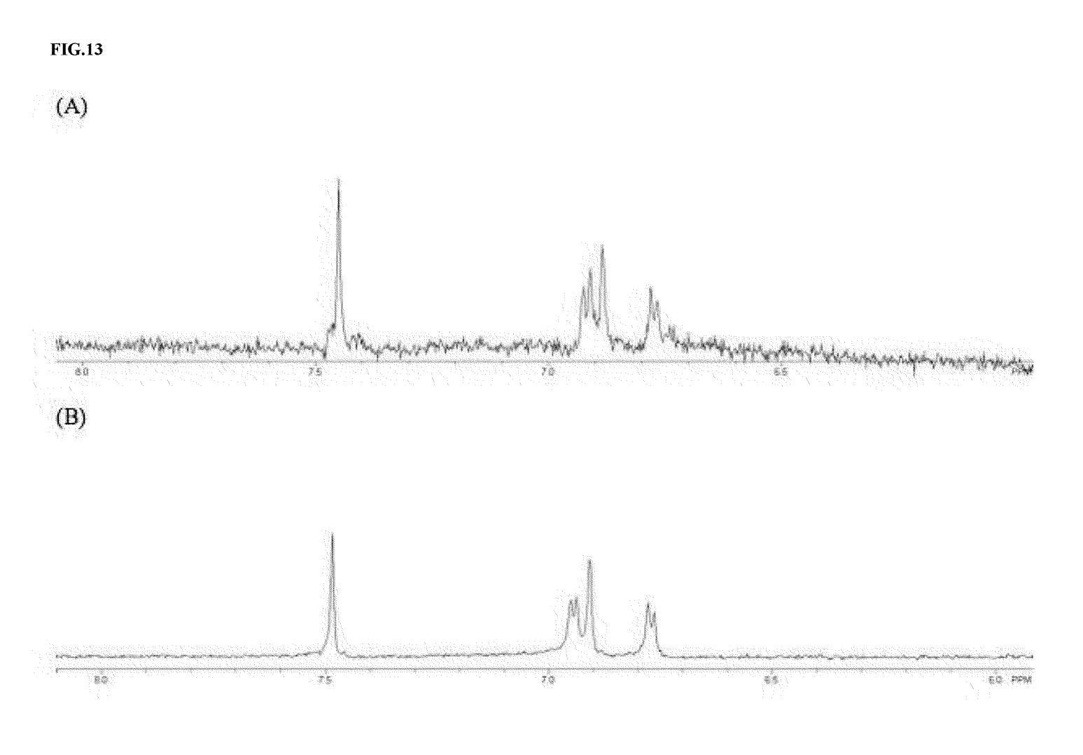

Synthesis of 2 (NHS-CRAA): (5-[(3,4-dihydroxyphenyl)methylene]-4-oxo-2-thioxo-3-thiazolidineacetic N-hydroxysuccinimide ester)⁵⁵. Under a N₂ atmosphere, a mixture of 6.22 g 1, 5.75 g N-hydroxysuccinimide, 20.6 g DCC, 50 mL DMSO and a small amount of DMAP catalyst was reacted at room temperature overnight. The next day the reaction was monitored by NMR (FIG. 12) and with TLC (using EMD Silica gel 60 F₂₅₄ developed with chloroform/methanol/acetic acid 12:3:1 v/v/v), visualized using a 254 nm UV light (the R_{f} of 1 is 0.39 and the new spot's R_{f} is 0.68). The DCU (dicyclohexylureo) was vacuum filtered off and the NHS-CRAA (2, FIG. 2) DMSO solution was used in the next step without further purification. The methylene protons (Ha) undergo significant chemical shift change (to Hb) that permits monitoring of formation of the NHS ester from CRAA. After 96 hours, yield was 20% as the NHS ester (with remainder as unreacted CRAA). Even after an additional 4 days, there were no further spectral changes.

Synthesis of CRAA (1) agarose matrix (5-[(3,4-dihydroxyphenyl)methylene]-4-oxo-2-thioxo-3-thiazolidineacetic ω-aminohexyl-agarose amide)⁵⁶. NHS-CRAA ester (2) DMSO solution was added dropwise into 100 mL ω-aminohexyl-agarose suspended in 600 mL 100 mM phosphate buffer, pH 10.0. During this process, the pH was maintained at 10.0, and then the reaction was run at 7° C. in a refrigerator overnight. The next day, 60 mL 1 M Tris-HCl buffer, pH 6.5 was added to the reaction mixture to stop the reaction. Then 47.7 g sodium chloride was added to form a final 0.5 M saline solutions. The liquid layer was decanted and the labeled matrix (FIG. 2) was washed with a large amount of deionized water. Then ˜10 mL matrix was packed into a 1×20 cm column for column chromatography.

NHS-CRAA ester (1) in in-cell uptake and labeling study⁵⁷. E. coli containing the pET11a DHPR expression construct was inoculated into 30 mL LB culture medium, growing overnight at 37° C., 225 rpm. The next day, 10 mL of this culture was added to two flasks (flasks A and B, each containing 800 mL LB medium with 50 μg/mL carbenicillin). The OD₆₀₀ was monitored until it reached ˜1.0. To flask A was added 0.8 mL of a 0.4 M IPTG stock to start induction⁵⁸. Flask B was used as a control without induction. 5 hours later, cells were collected (centrifuged 10 minutes at 4,000 rpm) and washed once with 100 mM PBS buffer, pH 7.4. The Cells were then suspended in 100 mL of pH 7.4 PBS buffer (8 g NaCl, 0.2 g KCl, 1.44 g Na₂HPO₄, 0.24 g KH₂PO₄ in 1.0 L) and incubated for 5 minutes. Then, 1 mL NHS-CRAA ester (2) was added to each flask and incubated at room temperature for about 30 minutes (FIG. 3a). 10 mL of 1 M Tris-HCl, pH 6.5, was added to each flask and shaken for another 5 minutes, to quench the reaction. The cells were collected again by centrifuging and washed twice with PBS buffer. The cells were lysed with SDS loading buffer (50 mM Tris-HCl (pH 6.8), 100 mM dithiothreitol, 2% SDS, 0.1% bromophenol blue, 10% glycerol) and were run on a 4-12% Bis-Tris SDS-PAGE gel (FIG. 3b). The gel was fluorescently imaged on a Kodak Image Station, to selectively detect 1 which has λₘₐₓ for absorbance and emission at 465 nm and 535 nm³⁷. Cells were fluorescently imaged in PBS buffer after the wash (before lysis) using 495 nm/520 nm excitation/emission filters on the fluorescence microscope. Complementary fluorescence and bright field images may be shifted slightly relative to each other, because of bacterial motion between image captures. Exposure time was 1/3.5 sec, and 1000× magnification was used (FIG. 4). Fluorescence images of control cells (no IPTG treatment; no DHP) indicate much less labeling of cells, with the majority of the fluorescence coming from cellular debris (FIG. 9, as the fluorescence does not correlate with the cells observed in the bright filed image (Panel B), which is more apparent in the overlay of both images (Panel C)).

General procedure for CRAA (1) affinity column chromatography and target fishing³². The CRAA (1) affinity column was equilibrated with buffer A, which contains 25 mM Tris-HCl, 50 mM NaCl and 0.1% NaN₃, pH 7.8. Washing was done until the eluent was nearly colorless (1 is intensely colored). Then, the protein sample (E. coli, M. tuberculosis or human liver) was loaded onto the affinity column and washed with a large amount of buffer A until no protein sample was detected using a Bradford assay (BioRad). The buffer volume used was usually 10-fold of the packing volume of the column. Then the affinity column was eluted with buffer B, which is the same as buffer A except containing 4 mM 1. Fractions were collected, then separated on an SDS-PAGE gel and stained using a SilverQuest kit. Fractions from E. coli and M. tuberculosis were compared, and showed very similar banding profiles based on SDS-PAGE gel analysis (FIG. 6a). Human liver and M. tuberculosis fractions were either used directly for mass spectral analysis (next section), or were separated using SDS-PAGE, with protein extracted from the bands indicated in FIG. 6c.

Sample Preparation for Mass Spectrometry. Pooled fractions, after elution from the CRAA (1) affinity column, were concentrated using a Centricon filter with 10 kDa cutoff (Millipore). Then, 100 μl of affinity purified protein mixtures were polymerized in the presence of 100 μl acrylamide/bis(30% T/2.67% C), 2 μl of 10% ammonium persulfate and 2 μl TEMED. With this mixture a 15% gel piece was formed. Polymerization was performed in the cap of an Eppendorf tube. The polymerized gel pieces were then transferred to the corresponding Eppendorf tube in 1 ml of 40% methanol, 7% acetic acid and incubated for 30 minutes. The gel pieces were washed twice in water for 30 min each time while sonicating. Gel pieces were then washed twice in 50% acetonitrile for 30 min each time while sonicating. The gel pieces were then washed twice again, this time in 50% acetonitrile in 50 mM ammonium bicarbonate, pH 8.0. The gel pieces were then dried using a speed vac from Savant. To each gel piece was added 200 μl of 20 mM ammonium bicarbonate, pH 8.0, containing 1 μg trypsin (Promega); this was incubated overnight at 37° C. Each gel piece with the digested proteins was then extracted twice with 70% acetonitrile in 0.1% formic acid. From this step on all water used was MS quality water. Corresponding extracts of each gel were pooled together and dried. To each dried sample was added 6M guanidine-HCl in 5 mM potassium phosphate and 1 mM DTT, pH 6.5. This was sonicated and peptides were extracted using a C₁₈ ZipTip from Millipore. Extracted peptides were then collected into an insert in a vial to be used for mass spectrometry, and dried in the inserts. To each dried sample was added 5 μl of 0.1% formic acid in MS water containing 5% acetonitrile. Samples were then ready for mass spectrometry, and were injected into the LTQ LC/MS. The MS/MS data were collected and searched against the appropriate subset of the Uniprot database.

¹H NMR STD (saturation transfer difference) spectra for CRAA binding to either malate dehydrogenase (MDH) (FIG. 13A) or glutamate dehydrogenase (FIG. 13B) establishes that the CRAA scaffold binds proteins that were identified using the proteomic assay, thereby validing the method for identifying non-target proteins. Glutamate dehydrogenase (bovine liver, GDH) and malate dehydrogenase (porcine heart) were from Sigma Aldrich. Both experiments were performed on a 600 MHz Varian NMR system at 25° C. using the cyclonecyclnoe pulse sequence (a steady state NOE experiment) with 256 scans. On and off resonance irradiations were at 0.5 and −11 ppm respectively, performed for 4 seconds each. Since the spectra shown are difference spectra (on vs. off resonance irradiation of protein), they indicate that CRAA binds to protein. (See FIG. 13). In both cases, CRAA was present at 3 mM and the binding study was done in a 25 mM phosphate buffer at pH 7.6 with 250 ug GDH or 166 ug MDH in a total volume of 0.6 mLs. The use of STD assays in functional proteomic binding studies has been described (Yao and Sem, FEBS Lett. (2005) 579, 661-666, the content of which is incorporated herein by reference in its entirety). To verify that binding had occurred in the cofactor binding site, competition studies were also performed (not shown). Addition of 1 mM NADH to the MDH sample caused a decrease in STD signal for CRAA (3 mM) of 35%. The analogous competition with GDH (using NADP+ as competitor) produced no change in STD signal for CRAA—which might be due to higher affinity for CRAA than to NADP+. To test this hypothesis, the competition was reversed where 3 mM NADP+ STD signal was monitored before and after adding CRAA (1 mM). A decrease in the NADP+ STD signal of 50% was now observed, due to CRAA addition.

Elution of human liver proteins from the CRAA- and acetylamide-control resins using free CRAA to elute This experiment served as a control for the experiment of FIG. 6. Resin was used either as is (with no ligand attached to the ω-aminohexyl group), or after covalent addition of an acetyl group to make CH₃C(O)NH-hexyl-agarose. After loading human liver sample, the column was washed with 50 mL buffer A until no protein was present. Then, the column was eluted with 50 mL buffer B, and fractions were collected. In the SDS-PAGE gel, the combined wash-through and elution fractions were loaded in one lane; it should reflect the whole pattern of separation, for flow through versus CRAA elution. Buffer A, 25 mM Tris-HCl, pH 7.8 with 0.01% NaN₃ and 50 mM NaCl. Buffer B, 25 mM Tris-HCl with 0.01% NaN₃, 50 mM NaCl and 4 mM CRAA, pH 7.8. (See FIG. 14). Notably, there is no protein that was bound to the resin and then later eluted by CRAA in the control resin, and for the resin that had the positively charge w-amino group, one protein band is observed (this apparently was acting like an anion exchange resin; the more relevant control for FIG. 6 though is in Lane 6).

Abbreviations

ADME, Absorption, Distribution, Metabolism, and Excretion; CRAA, Catechol-Rhodanine Acetic Acid; DCC, N,N′-Dicyclohexylcarbodiimide; DCU, Dicyclohexylurea; DHPR, Dihydrodipicolinate Reductase; DMAP, 4-(Dimethylamino)pyridine; ESI, Electrospray Ionization; HPLC, High Performance Liquid Chromatography; IPTG, Isopropyl β-D-1-thiogalactopyranoside; LC-MS, Liquid Chromatography-Mass Spectrometry; LTQ, Linear Trap Quadrupole; NHS, N-hydroxysuccinimide ester; NMR-SOLVE, Structurally Oriented Library Valency Engineering; PAGE, Polyacrylamide Gel Electrophoresis; PBS, Phosphate Buffered Saline; SDS, Sodium Dodecyl Sulfate; TB, Tuberculosis; Tris, Tris(hydroxymethyl)aminomethane; and TEMED, tetramethylethylenediamine.

### Example 2

### Chemical Proteomic Assay of Brain Proteins Interacting with Drug Lead Molecules: Application of Proteomic Assay Using Tandem Mass Spectroscopy to Identify Proteins that Bind to DMP543, a KCNQ Channel Blocker

The goal of this proteomic assay is to identify proteins that interact with DMP543, which previously has been shown to activate the KCNQ potassium channel (Zaczek et al.⁵⁹) and thereby mediating, at least, some of the desired therapeutic effects of these channels. It is possible that DMP543 is also interacting with other brain proteins. In fact, it has been shown that XE991, a very close congener compound to DMP543, binds to and blocks the activity of ERG potassium channels that are also expressed by neurons (Elmedyb et al., 2007⁶⁰). In addition, linopirdine, another close congener of DMP543, at concentrations above 10 μM, blocks several other potassium currents including the transient outward current (I_{A}), the delayed rectifier current (I_{K}), the after-hyperpolarization currents (I_{AHP}), the inward rectifier current (I_{Q}), and the potassium leak current (I_{L}) (Schnee and Brown, 1998⁶¹). Further, the heart muscle cells express a potassium channel made up of KCNQ1 and minK (KCNE1) subunits which constitutes the cardiac delayed rectifier potassium current and regulates QT interval in the ECG. XE991 blocks the activity of this channel with K_{D}=11.1±1.8 μM (Wang et al., (2000)⁶²). A significant blockade of the heart muscle potassium channel may increase the risk for congenital cardiac disorder known as long QT syndrome that can lead to ventricular arrhythmias and sudden death (Wang et al., (1996)⁶³). These off-target interactions with other proteins increase the risk for emergence of undesirable side-effects after extended exposure to the drug. There might be other proteins interacting with DMP543 that may either contribute to the behavioral effects or may underlie undesirable side effects. At this time, the identity of these proteins is unknown. Identification of these proteins will allow design of improved drug leads with significantly decreased off-target interactions, and more selectivity at the KCNQ target; thereby, reduce risk for side effects.

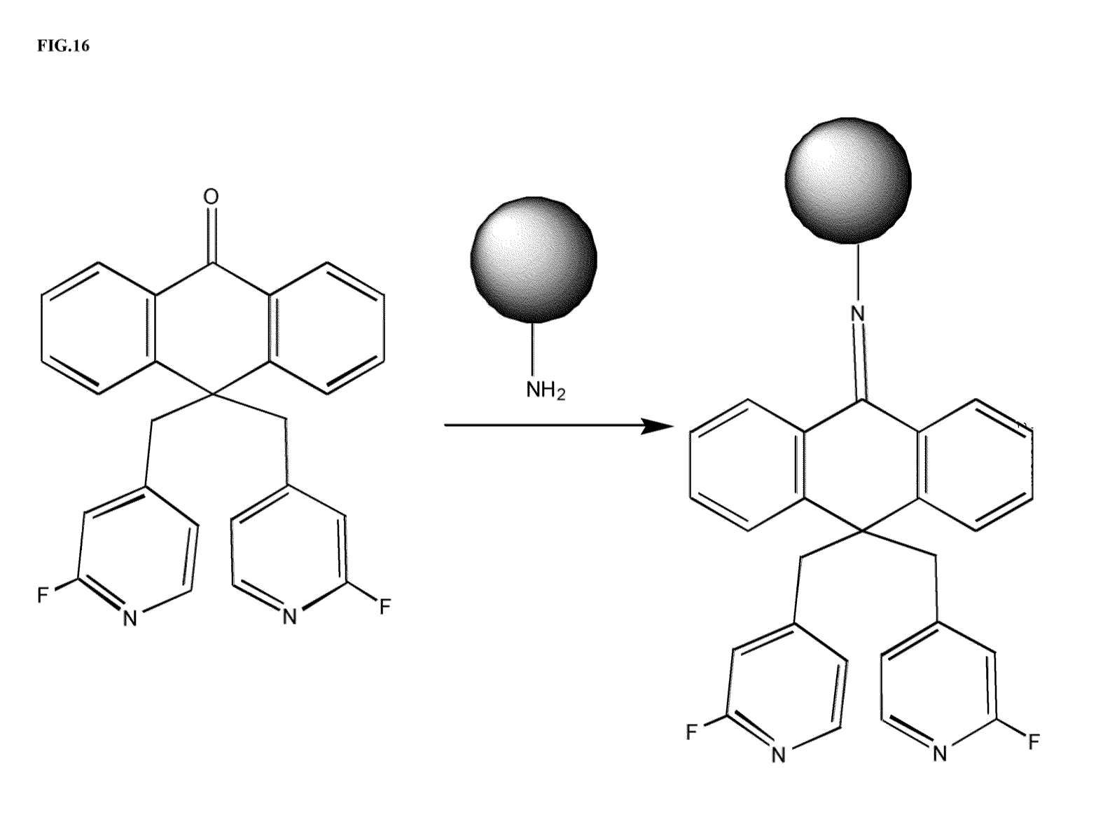

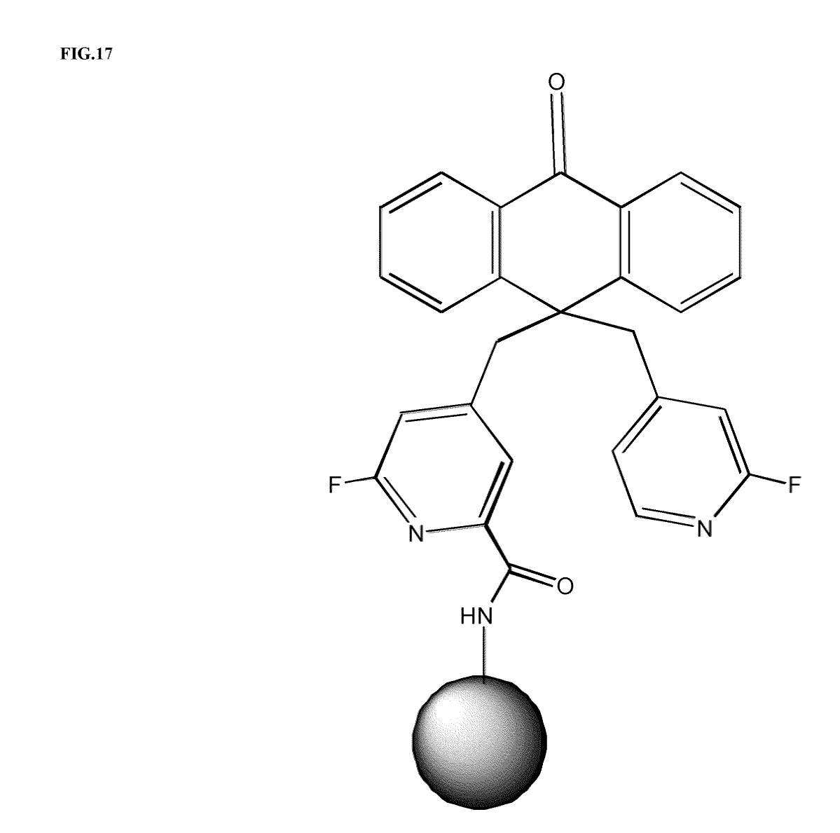

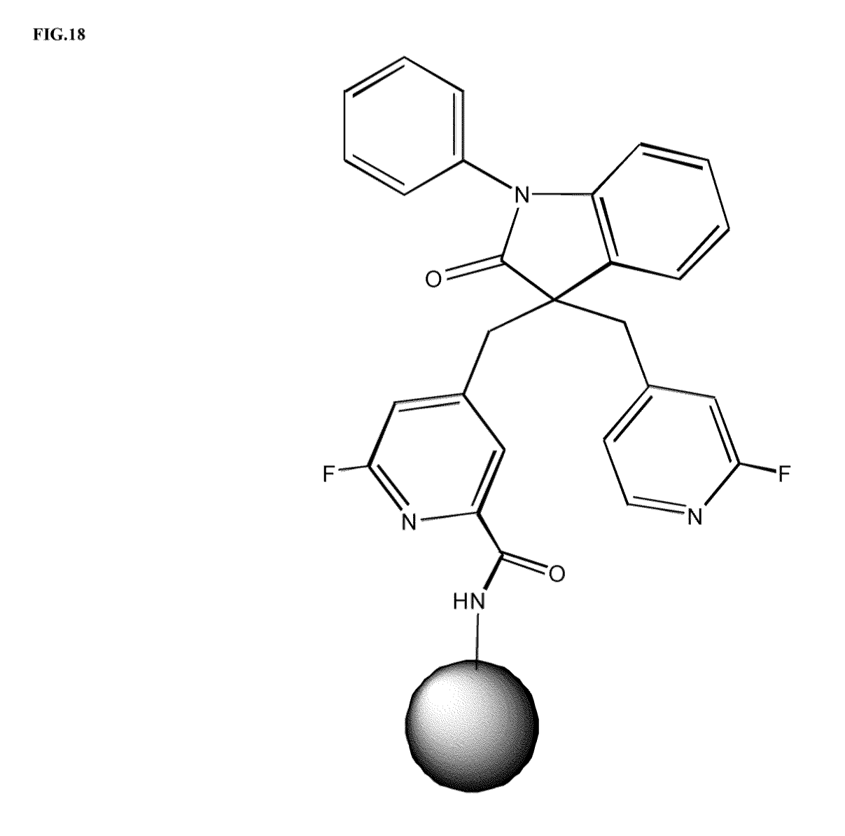

This approach is called chemical proteomic “fishing”, and is becoming increasingly useful as a way to assess off-target binding of drugs (Ge et al., (2008)⁶⁴; Peters and Gray, 2007; Sleno and Emili, (2008)⁶⁵) (FIG. 5). In this method, DMA will be covalently tethered to an affinity resin (Zaczek et al.⁵⁹, 1998; Earl et al., (1998)⁶⁶; Pest et al., (2000)⁶⁷). The simplest attachment strategy will be to tether the drug lead ligand via its ketone functionality, using a ω-aminohexyl agarose resin, by forming an imine linkage, as shown in FIG. 16. But other linkages of DMP543 to the resin are possible. In particular, since the synthetic precursor used to prepare DMP543 is 2-fluoro-4-methylpyridine, an alternative pyridyl synthetic precursor with functionalities that permit other attachment may be utilized. For example, an acid functionality at the 6-position would permit linkage to the ω-aminohexyl agarose resin using N-hydroxysuccinimide/DCC activation to form an amide linkage, as described previously (FIG. 17) (Ge et al., (2008)⁶⁴).

Prepared as such, the affinity resin may be used to purify and subsequently identify rat brain and heart muscle proteins that bind to the DMP543 lead molecule. The heart muscle protein screening may be used to identify and confirm the KCNQ1/minK potassium channel as a target for DMP543. For brain tissue studies, homogenate of membrane-bound proteins from frontal cortex, hippocampus, and striatum tissues may be prepared. These tissues may be suitable because of their suggested role in schizophrenia. In addition, all three of these tissues express high levels of KCNQ potassium channels (Tam et al., (1991)⁶⁸; Saganich et al., (2001)⁶⁹). Therefore, screening methods utilizing these tissues can corroborate the interaction of DMP543-KCNQ potassium channels and further may identify off-target proteins that interact with DMP543 that might be important in the role of the KCNQ channel in schizophrenia. The homogenization method and buffer may be prepared as previously described (Tam, (1983)⁷⁰; Tam et al., (1991)⁷⁰; Meyers and Kritzer, (2009)⁷¹). The protein homogenate will be loaded onto the DMP543 affinity column. After washing the column with buffer solution, the drug-binding proteins will be eluted with a solution of free DMP543 molecule at a concentration of 0.1-2 mM, fractions will be collected and protein content of each fraction will be characterized using SDS/PAGE gel analysis, as shown in FIG. 6. Protein bands in the SDS/PAGE gels may be excised and the proteins extracted for tandem mass spectrometry analysis, as shown in FIG. 5. This process will identify the proteins that are present in each fraction. Suitable analysis methods have been described (Ge et al., (2008)⁶⁴).

In these experiments, a proteomic assay is used to determine the set of proteins in brain tissue that bind to DMP543. The proteomic assay may be repeated utilizing heart tissue or liver tissue. Accordingly a binding profile or proteomic profile is generated for DMP543. The results of these experiments may be utilized to identify and optimize other drugs with increased specificity for the KCNQ target than DMP543. For example, an improved drug lead would elute only KCNQ2-5 proteins from a column utilized in the assay, but significantly fewer or no other off-target proteins that bound the original DMP543 molecule. Compounds may be assessed based on binding affinity to KCNQ channel in the brain tissue, lack of binding to non-brain analogs of the KCNQ channel (e.g., the heart muscle KCNQ1/mink channel), and fewest bound off-target proteins.

### Example 3

### Method of Quantitatively Comparing Proteomic Profiles

Referring to FIG. 22, after densitometric scanning of a gel, the data correlating position on a gel (the x-axis in panel A) and protein band intensity (the y-axis in panel A) can be plotted as shown in Panel A for two different proteomic profiles. Optionally, the relative intensities of profiles can be scaled. However, alternatively, correlation analysis (as implemented in the software “R”) can be utilized. In panel B is plotted the correlation between the two proteomic profiles in panel A, again as a function of position on the gel (on the x-axis). A correlation value of 1.0 means a perfect match. Thus, there are differences at positions 700 and 800 (arbitrary units—derived from pixels). “Similarity” between two proteomic profiles can be assessed or quantified based on average correlation. For example, if the proteomic profile of tissue proteins eluted from an affinity column (containing drug-1 covalently attached) by eluting with a solution of drug-1 (which is know to be active against disease-1) matched the profile when the same column and tissue sample is eluted with compound 2, with an average correlation value of >0.7, this would identify compound 2 as being useful for treating disease-1. Even more preferred would be an average correlation function of >0.8, or >0.9.

### REFERENCES FOR EXAMPLES

1. Fitzgerald, G. A. Opinion: anticipating change in drug development: the emerging era of translational medicine and therapeutics. Nat. Rev. Drug Discov. 2005, 4, 815-818.
2. Tolliday, N.; Clemons, P. A.; Ferraiolo, P.; Koehler, A. N.; Lewis, T. A.; Li, X.; Schreiber, S. L.; Gerhard, D. S.; Eliasof, S. Small molecules, big players: the National Cancer Institute's Initiative for Chemical Genetics. Cancer Res. 2006, 66, 8935-8942.
3. Strausberg, R. L.; Schreiber, S. L. From knowing to controlling: a path from genomics to drugs using small molecule probes. Science 2003, 300, 294-295.
4. Krishnamurty, R.; Maly, D. J. Chemical genomic and proteomic methods for determining kinase inhibitor selectivity. Comb. Chem. High Throughput Screen. 2007, 10, 652-666.
5. Daub, H.; Godl, K.; Brehmer, D.; Klebl, B.; Muller, G. Evaluation of kinase inhibitor selectivity by chemical proteomics. Assay Drug Dev. Technol. 2004, 2, 215-224.
6. Peters, E. C.; Gray, N. S. Chemical proteomics identifies unanticipated targets of clinical kinase inhibitors. ACS Chem. Biol. 2007, 2, 661-664.
7. Argyrou, A.; Jin, L.; Siconilfi-Baez, L.; Angeletti, R. H.; Blanchard, J. S. Proteome-wide profiling of isoniazid targets in Mycobacterium tuberculosis. Biochemistry 2006, 45, 13947-13953.
8. Schalon, C.; Surgand, J. S.; Kellenberger, E.; Rognan, D. A simple and fuzzy method to align and compare druggable ligand-binding sites. Proteins 2008.
9. Hajduk, P. J.; Huth, J. R.; Tse, C. Predicting protein druggability. Drug Discov. Today 2005, 10, 1675-1682.
10. DeSimone, R. W.; Currie, K. S.; Mitchell, S. A.; Darrow, J. W.; Pippin, D. A. Privileged structures: applications in drug discovery. Comb. Chem. High Throughput Screen. 2004, 7, 473-494.
11. Duarte, C. D.; Barreiro, E. J.; Fraga, C. A. Privileged structures: a useful concept for the rational design of new lead drug candidates. Mini Rev. Med. Chem. 2007, 7, 1108-1119.
12. Costantino, L.; Barlocco, D. Privileged structures as leads in medicinal chemistry. Curr. Med. Chem. 2006, 13, 65-85.
13. Muller, G. Medicinal chemistry of target family-directed masterkeys. Drug Discov. Today 2003, 8, 681-691.
14. Bemis, G. W.; Murcko, M. A. The properties of known drugs. 1. Molecular frameworks. J. Med. Chem. 1996, 39, 2887-2893.
15. Brustle, M.; Beck, B.; Schindler, T.; King, W.; Mitchell, T.; Clark, T. Descriptors, physical properties, and drug-likeness. J. Med. Chem. 2002, 45, 3345-3355.
16. Peters, U.; Cherian, J.; Kim, J. H.; Kwok, B. H.; Kapoor, T. M. Probing cell-division phenotype space and Polo-like kinase function using small molecules. Nat. Chem. Biol. 2006, 2, 618-626.
17. Smith, A. B., 3rd; Favor, D. A.; Sprengeler, P. A.; Guzman, M. C.; Carroll, P. J.; Furst, G. T.; Hirschmann, R. Molecular modeling, synthesis, and structures of N-methylated 3,5-linked pyrrolin-4-ones toward the creation of a privileged nonpeptide scaffold. Bioorg. Med. Chem. 1999, 7, 9-22.
18. Amano, M.; Koh, Y.; Das, D.; Li, J.; Leschenko, S.; Wang, Y. F.; Boross, P. I.; Weber, I. T.; Ghosh, A. K.; Mitsuya, H. A novel bis-tetrahydrofuranylurethane-containing nonpeptidic protease inhibitor (PI), GRL-98065, is potent against multiple-PI-resistant human immunodeficiency virus in vitro. Antimicrob. Agents Chemother. 2007, 51, 2143-2155.
19. Guo, T.; Hobbs, D. W. Privileged structure-based combinatorial libraries targeting G protein-coupled receptors. Assay Drug Dev. Technol. 2003, 1, 579-592.
20. Sasse, B. C.; Mach, U. R.; Leppaenen, J.; Calmels, T.; Stark, H. Hybrid approach for the design of highly affine and selective dopamine D(3) receptor ligands using privileged scaffolds of biogenic amine GPCR ligands. Bioorg. Med. Chem. 2007, 15, 7258-7273.
21. Sem, D. S.; Bertolaet, B.; Baker, B.; Chang, E.; Costache, A. D.; Coutts, S.; Dong, Q.; Hansen, M.; Hong, V.; Huang, X.; Jack, R. M.; Kho, R.; Lang, H.; Ma, C. T.; Meininger, D.; Pellecchia, M.; Pierre, F.; Villar, H.; Yu, L. Systems-based design of bi-ligand inhibitors of oxidoreductases: filling the chemical proteomic toolbox. Chem. Biol. 2004, 11, 185-194.
22. Kho, R.; Newman, J. V.; Jack, R. M.; Villar, H. O.; Hansen, M. R. Genome-wide profile of oxidoreductases in viruses, prokaryotes, and eukaryotes. J. Proteome Res. 2003, 2, 626-632.
23. Kho, R.; Baker, B. L.; Newman, J. V.; Jack, R. M.; Sem, D. S.; Villar, H. O.; Hansen, M. R. A path from primary protein sequence to ligand recognition. Proteins 2003, 50, 589-599.
24. Godl, K.; Wissing, J.; Kurtenbach, A.; Habenberger, P.; Blencke, S.; Gutbrod, H.; Salassidis, K.; Stein-Gerlach, M.; Missio, A.; Cotten, M.; Daub, H. An efficient proteomics method to identify the cellular targets of protein kinase inhibitors. Proc. Natl. Acad. Sci. U.S.A. 2003, 100, 15434-15439.
25. Bantscheff, M.; Eberhard, D.; Abraham, Y.; Bastuck, S.; Boesche, M.; Hobson, S.; Mathieson, T.; Perrin, J.; Raida, M.; Rau, C.; Reader, V.; Sweetman, G.; Bauer, A.; Bouwmeester, T.; Hopf, C.; Kruse, U.; Neubauer, G.; Ramsden, N.; Rick, J.; Kuster, B.; Drewes, G. Quantitative chemical proteomics reveals mechanisms of action of clinical ABL kinase inhibitors. Nat. Biotechnol. 2007, 25, 1035-1044.
26. Poupaert, J.; Carato, P.; Colacino, E.; Yous, S. 2(3H)-benzoxazolone and bioisosters as “privileged scaffold” in the design of pharmacological probes. Curr. Med. Chem. 2005, 12, 877-885.
27. Matsuoka, K.; Sakamoto, N.; Akanuma, Y.; Hotta, N.; Shichiri, M.; Toyota, T.; Oka, Y.; Kawamori, R.; Shigeta, Y.; ADCT Study Group. A long-term effect of epalrestat on motor conduction velocity of diabetic patients: ARI-Diabetes Complication Trial. Diabetes Res. Clin. Pract. 2007, 77, S263-268.
28. Hotta, N.; Akanuma, Y.; Kawamori, R.; Matsuoka, K.; Oka, Y.; Shichiri, M.; Toyota, T.; Nakashima, M.; Yoshimura, I.; Sakamoto, N.; Shigeta, Y.; ADCT Study Group. Long-term clinical effects of Epalrestat, an aldose reductase inhibitor, on diabetic peripheral neuropathy. Diabetes Care 2006, 29, 1538-1544.
29. Bolton, J. L.; Pisha, E.; Shen, L.; Krol, E. S.; Iverson, S. L.; Huang, Z.; van Breemen, R. B.; Pezzuto, J. M. The reactivity of o-quinones which do not isomerize to quinone methides correlates with alkylcatechol-induced toxicity in human melanoma cells. Chem. Biol. Interact. 1997, 106, 133-148.
30. Hutzler, J. M.; Melton, R. J.; Rumsey, J. M.; Thompson, D. C.; Rock, D. A.; Wienkers, L. C. Chem. Res. Toxicol. 2008, in press.
31. Barry, S.; O'Carra, P. Affinity chromatography of nicotinamide-adenine dinucleotide-linked dehydrogenases on immobilized derivatives of the dinucleotide. Biochem. J. 1973, 135, 595-607.
32. Morelli, A.; Benatti, U. Simple chemical synthesis of a specific effector for the affinity chromatography of nicotinamide adenine dinucleotide phosphate-dependent dehydrogenases. Ital. J. Biochem. 1974, 23, 279-291.
33. Trayer, I. P.; Trayer, H. R. Affinity chromatography of nicotinamide nucleotide-dependent dehydrogenases on immobilized nucleotide derivatives. Biochem. J. 1974, 141, 775-787.
34. Ng, E.; Schriemer, D. C. Emerging challenges in ligand discovery: new opportunities for chromatographic assay. Expert Rev. Proteomics 2005, 2, 891-900.
35. Bertrand, R.; Derancourt, J.; Kassab, R. Fluorescence characterization of structural transitions at the strong actin binding motif in skeletal myosin affinity labeled at cysteine 540 with novel spectroscopic cysteaminyl mixed disulfides. Biochemistry 2000, 39, 14626-14637.
36. Scapin, G.; Reddy, S. G.; Zheng, R.; Blanchard, J. S. Three-dimensional structure of Escherichia coli dihydrodipicolinate reductase in complex with NADH and the inhibitor 2,6-pyridinedicarboxylate. Biochemistry 1997, 36, 15081-15088.
37. Ge, X.; Sem, D. S. Affinity-based chemical proteomic probe for dehydrogenases: fluorescence and visible binding assays in gels. Anal. Biochem. 2007, 370, 171-179.
38. Barglow, K. T.; Cravatt, B. F. Activity-based protein profiling for the functional annotation of enzymes. Nat. Methods 2007, 4, 822-827.
39. Evans, M. J.; Saghatelian, A.; Sorensen, E. J.; Cravatt, B. F. Target discovery in small-molecule cell-based screens by in situ proteome reactivity profiling. Nat. Biotechnol. 2005, 23, 1303-1307.
40. Dye, C.; Scheele, S.; Dolin, P.; Pathania, V.; Raviglione, M. C. Consensus statement. Global burden of tuberculosis: estimated incidence, prevalence, and mortality by country. WHO Global Surveillance and Monitoring Project. JAMA 1999, 282, 677-686.
41. Okada, M.; Kobayashi, K. Recent progress in mycobacteriology. Kekkaku 2007, 82, 783-799.
42. Hori, T.; Ishijima, J.; Yokomizo, T.; Ago, H.; Shimizu, T.; Miyano, M. Crystal structure of anti-configuration of indomethacin and leukotriene B4 12-hydroxydehydrogenase/15-oxo-prostaglandin 13-reductase complex reveals the structural basis of broad spectrum indomethacin efficacy. J. Biochem. 2006, 140, 457-466.
43. Clish, C. B.; Sun, Y. P.; Serhan, C. N. Identification of dual cyclooxygenase-eicosanoid oxidoreductase inhibitors: NSAIDs that inhibit PG-LX reductase/LTB(4) dehydrogenase. Biochem. Biophys. Res. Commun. 2001, 288, 868-874.
44. Mills, P. B.; Struys, E.; Jakobs, C.; Plecko, B.; Baxter, P.; Baumgartner, M.; Willemsen, M. A.; Omran, H.; Tacke, U.; Uhlenberg, B.; Weschke, B.; Clayton, P. T. Mutations in antiquitin in individuals with pyridoxine-dependent seizures. Nat. Med. 2006, 12, 307-309.
45. Bok, L. A.; Struys, E.; Willemsen, M. A.; Been, J. V.; Jakobs, C. Pyridoxine-dependent seizures in Dutch patients: diagnosis by elevated urinary alpha-aminoadipic semialdehyde levels. Arch. Dis. Child. 2007, 92, 687-689.
46. Salomons, G. S.; Bok, L. A.; Struys, E. A.; Pope, L. L.; Darmin, P. S.; Mills, P. B.; Clayton, P. T.; Willemsen, M. A.; Jakobs, C. An intriguing “silent” mutation and a founder effect in antiquitin (ALDH7A1). Ann. Neurol. 2007, 62, 414-418.
47. Eirich, L. D.; Vogels, G. D.; Wolfe, R. S. Distribution of coenzyme F420 and properties of its hydrolytic fragments. J. Bacteriol. 1979, 140, 20-27.
48. Eirich, L. D.; Vogels, G. D.; Wolfe, R. S. Proposed structure for coenzyme F420 from Methanobacterium. Biochemistry 1978, 17, 4583-4593.
49. Purwantini, E.; Daniels, L. Molecular analysis of the gene encoding F420-dependent glucose-6-phosphate dehydrogenase from Mycobacterium smegmatis. J. Bacteriol. 1998, 180, 2212-2219.
50. Salvetti, S.; Celandroni, F.; Ghelardi, E.; Baggiani, A.; Senesi, S. Rapid determination of vitamin B2 secretion by bacteria growing on solid media. J. Appl. Microbiol. 2003, 95, 1255-1260.
51. Messinger, J.; Hirvela, L.; Husen, B.; Kangas, L.; Koskimies, P.; Pentikainen, O.; Saarenketo, P.; Thole, H. New inhibitors of 17beta-hydroxysteroid dehydrogenase type 1. Mol. Cell. Endocrinol. 2006, 248, 192-198.
52. Rizner, T. L.; Zakelj-Mavric, M. Characterization of fungal 17beta-hydroxysteroid dehydrogenases. Comp. Biochem. Physiol. B. Biochem. Mol. Biol. 2000, 127, 53-63.
53. Purkayastha, A.; McCue, L. A.; McDonough, K. A. Identification of a Mycobacterium tuberculosis putative classical nitroreductase gene whose expression is coregulated with that of the acr gene within macrophages, in standing versus shaking cultures, and under low oxygen conditions. Infect. Immun. 2002, 70, 1518-1529.
54. Srivastava, V.; Rouanet, C.; Srivastava, R.; Ramalingam, B.; Locht, C.; Srivastava, B. S. Macrophage-specific Mycobacterium tuberculosis genes: identification by green fluorescent protein and kanamycin resistance selection. Microbiology 2007, 153, 659-666.
55. Vanin, E. F.; Ji, T. H. Synthesis and application of cleavable photoactivable heterobifunctional reagents. Biochemistry 1981, 20, 6754-6760.
56. Witzemann, V.; Muchmore, D.; Raftery, M. A. Affinity-directed cross-linking of membrane-bound acetylcholine receptor polypeptides with photolabile alpha-bungarotoxin derivatives. Biochemistry 1979, 18, 5511-5518.
57. Waugh, S. M.; DiBella, E. E.; Pilch, P. F. Isolation of a proteolytically derived domain of the insulin receptor containing the major site of cross-linking/binding. Biochemistry 1989, 28, 3448-3455.
58. Reddy, S. G.; Sacchettini, J. C.; Blanchard, J. S. Expression, purification, and characterization of Escherichia coli dihydrodipicolinate reductase. Biochemistry 1995, 34, 3492-3501.
59. Zaczek R, Chorvat R J, Saye J A, Pierdomenico M E, Maciag C M, Logue A R, Fisher B N, Rominger D H, Earl R A (1998) Two new potent neurotransmitter release enhancers, 10,10-bis(4-pyridinylmethyl)-9(10H)-anthracenone and 10,10-bis(2-fluoro-4-pyridinylmethyl)-9(10H)-anthracenone: comparison to linopirdine. J. Pharmacol. Exp. Ther. 285, 724-730.
60. Elmedyb P, Calloe K, Schmitt N, Hansen R S, Grunnet M, Olesen S P (2007) Modulation of ERG channels by XE991. Basic & Clinical Pharmacol. & Toxicol. 100, 316-322.
61. Schnee M E, Brown B S (1998) Selectivity of linopirdine (DuP 996), a neurotransmitter release enhancer, in blocking voltage-dependent and calcium-activated potassium currents in hippocampal neurons. J. Pharmacol. Exp. Ther. 286, 709-717.
62. Wang H S, Brown B S, McKinnon D, Cohen I S (2000) Molecular basis for differential sensitivity of KCNQ and I(Ks) channels to the cognitive enhancer XE991. Mol. Pharmacol. 57, 218-1223.
63. Wang Q, Curran M E, Splawski I, Burn T C, Millholland J M, VanRaay T J, Shen J, Timothy K W, Vincent G M, de Jager T, Schwartz P J, Toubin J A, Moss A J, Atkinson D L, Landes G M, Connors T D, Keating M T (1996) Positional cloning of a novel potassium channel gene: KVLQT1 mutations cause cardiac arrhythmias. Nat. Genet. 12, 17-23.
64. Ge X, Wakim B, and Sem D S (2008) Chemical proteomics-based drug design: target and antitarget fishing with a catechol-rhodanine privileged scaffold for NAD(P)(H) binding proteins. J. Med. Chem., 51, 4571-4580.
65. Sleno L, and Emili A (2008) Proteomic methods for drug target discovery. Curr. Opin. Chem. Biol. 12, 46.
66. Earl R A, Zaczek R, Teleha C A, Fisher B N, Maciag C M, Marynowski M E, Logue A R, Tam S W, Tinker W J, Huang S M, Chorvat R J (1998) 2-Fluoro-4-pyridinylmethyl analogues of linopirdine as orally active acetylcholine release-enhancing agents with good efficacy and duration of action. J. Med. Chem. 41, 4615-4622.
67. Pest J A, Huhn G F, Yin J, Xing Y, Fortunak J M, and Earl R A (2000) Efficient pyridinylmethyl functionalization: synthesis of 10, 10-Bis[(2-fluoro-4-pyridinyl)methyl]-9(10H)-anthracenone (DMP 543), an acetylcholine release enhancing agent. J. Org. Chem. 65, 7718-7722.
68. Tam S W, Rominger D, Nickolson V J (1991) Novel receptor site involved in enhancement of stimulus-induced acetylcholine, dopamine, and serotonin release. (1991) Mol. Pharmacol. 40, 16-21.
69. Saganich M J, Machado E, Rudy B (2001) Differential expression of genes encoding subthreshold-operating voltage-gated K+ channels in brain. J. Neurosci. 21, 4609-4624.
70. Tam S W (1983) Naloxone-inaccessible sigma receptor in rat central nervous system. Proc. Natl. Acad. Sci. 80, 6703-6707.
71. Meyers B and Kritzer M F (2009) In vitro binding assays using (3)H nisoxetine and (3)H WIN 35,428 reveal selective effects of gonadectomy and hormone replacement in adult male rats on norepinephrine but not dopamine transporter sites in the cerebral cortex. Neuroscience 159, 271-282.
72. Assadi-Porter F M, Tonelli M, Maillet E, Hallenga K, Benard O, Max M, Markley J L (2008) Direct NMR detection of the binding of functional ligands to the human sweet receptor, a heterodimeric family 3 GPCR. J. Am. Chem. Soc. 130, 7212-7213.
73. Bakshi V P, Geyer M A (1997) Phencyclidine-induced deficits in prepulse inhibition of startle are blocked by prazosin, an alpha-1 noradrenergic antagonist. J. Pharmacol. Exp. Ther. 283, 666-674.
74. Brown D A, Adams P R (1980) Muscarinic suppression of a novel voltage-sensitive K+ current in a vertebrate neurone. Nature 283, 673-676.
75. Fenton W S, McGlashan TH (1991) Natural history of schizophrenia subtypes. II. Positive and negative symptoms and long-term course. Arch. Gen. Psychiatry 48, 978-986.
76. Hajduk P J, and Greer J (2007) A decade of fragment-based drug design: strategic advances and lessons learned. Nature Reviews Drug Disc. 6, 21.1-219.
77. Lieberman J A, Stroup T S, McEvoy J P, Swartz M S, Rosenheck R A, Perkins D O, Keefe R S, Davis S M, Davis C E, Lebowitz B D, Severe J, Hsiao J K; Clinical Antipsychotic Trials of Intervention Effectiveness (CATIE) Investigators (2005) Effectiveness of antipsychotic drugs in patients with chronic schizophrenia. N. Engl. J. Med. 353, 1209-1223.
78. Mayer M, and Meyer B (2001) Group epitope mapping by saturation transfer difference NMR to identify segments of a ligand in direct contact with a protein receptor. J. Am. Chem. Soc. 123, 6108-6117.
79. Pellecchia M, Sem D S, and Wuthrich K. (2002) NMR in drug discovery. Nat. Rev. Drug Discov. 1, 211-219.
80. Sem D S. (1999) NMR-SOLVE Method for Rapid Ident. of Bi-Ligand Drug. U.S. Pat. No. 6,333,149 B1 ed., US (issued).
81. Sem D S, Yu L, Coutts S M, and Jack R. (2001) An Object-oriented Approach to Drug Design Enabled by NMR SOLVE, the First Real-Time Structural Tool for Characterizing Protein-Ligand Interactions. J. Cellular Biochemistry 37, S99-105.
82. Sem D S, Pellecchia M, Dong Q, Kelly M, Lee M S (2003) NMR Assembly of Chemical Entities. 20030113751 A1, US (pending).
83. Sem D S (2006) Fragment-based Approaches in Drug Discovery (Jahnke and Erlanson, Ed.), pp 163-196.
84. Shuker S B, Hajduk P J, Meadows R P, Fesik S W (1996) Discovering high-affinity ligands for proteins: SAR by NMR. Science. 274, 1531-1534.
85. Wang H S, Pan Z, Shi W, Brown B S, Wymore R S, Cohen I S, Dixon J E, McKinnon D (1998) KCNQ2 and KCNQ3 potassium channel subunits: molecular correlates of the M-channel. Science 282, 1890-1983.
86. Yao H, and Sem D S (2005) Cofactor fingerprinting with STD NMR to characterize proteins of unknown function: identification of a rare cCMP cofactor preference. FEBS Letters 579, 661-666.
87. Yao H, Stuart R, Cai S, and Sem, D S (2008) Structural characterization of the transmembrane domain from Subunit e (Su e) of yeast F₁F₀-ATP synthase: a helical GXXXG motif located just under the micelle surface. Biochemistry 47, 1910-1917.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

TABLE 1
Mass spectrometry-based identification of proteins
identified in the target fishing study using the
CRAA affinity column - human liver proteome*

| Accession | | Molecular | | |
|---|---|---|---|---|
| number | Annotated function | Weight | Percent | |
| (gi) | (Human) | (Da) | Coverage | Score |
| 6648067 | Malate dehydrogenase, | 35,531 | 52 | 1557 |
| | mitochondrial precursor | | | |
| 1346343 | Keratin, type II | 66,018 | 23 | 852 |
| | cytoskeletal 1 | | | |
| 113611 | Fructose-bisphosphate | 39,473 | 24 | 643 |
| | aldolase B | | | |
| | (Liver-type aldolase) | | | |
| 118504 | Aldehyde dehydrogenase, | 56,381 | 28 | 526 |
| | mitochondrial precursor | | | |
| 118541 | Glutamate dehydrogenase 1, | 61,398 | 29 | 523 |
| | mitochondrial precursor | | | |
| 118495 | Retinal dehydrogenase 1 | 54,862 | 22 | 377 |
| | (Aldehyde dehydrogenase | | | |
| | family 1 member A1) | | | |
| 81175178 | Keratin, type I | 62,129 | 30 | 346 |
| | cytoskeletal 9 | | | |
| | (Cytokeratin-9) (CK-9) | | | |
| | (Keratin-9) (K9) | | | |
| 59802911 | 10-formyltetrahydrofolate | 98,829 | 15 | 252 |
| | dehydrogenase (Aldehyde | | | |
| | dehydr. 1 family member L1) | | | |

*Pyridine nucleotide (NAD(P)(H)) binding proteins are indicated in bold. Annotation is directly from the database.

TABLE 2
Mass spectrometry-based identification of proteins identified in the target
fishing study using the CRAA affinity column - M. tuberculosis proteome¹

| Accession | | Molecular | | |
|---|---|---|---|---|
| number | Annotated function | weight | Percent | |
| (gi) | (Mycobacterium tuberculosis) | (Da) | Coverage | Score |
| 81671721 | Possible Oxidoreductase² | 33,220 | 30 | 80 |
| 54036852 | Chaperone protein clpB | 92,568 | 15 | 31 |
| 2829534 | Riboflavin biosynthesis protein ribD | 35,366 | 8 | 26 |
| | [Includes: Diaminohydroxyphosphoribosyl- | | | |
| | aminopyrimidine deaminase] | | | |
| 1706274 | Bifunctional enzyme cysN/cysC [Includes: | 67,839 | 3 | 20 |
| | Sulfate adenylyltransferase subunit 1] | | | |
| 81671959 | Putative uncharacterized protein³ | 30,296 | 30 | 17 |
| 81340808 | Putative uncharacterized protein⁴ | 38,520 | 16 | 13 |

¹Pyridine nucleotide (NAD(P)(H)) binding proteins are indicated in bold. Annotation is directly from the database. Of the M. tuberculosis nucleotide (NAD(P)(H)) binding proteins indicated, two have homologs in E. coli: gi81671721 is similar to the E. coli protein gi75240619 (Mr = 42,233 g/mol), and gi2829534 is similar to the E. coli protein gi75230139 (Mr = 39,456 g/mol).
²A subsequent NCBI search indicates this protein has homology to Coenzyme F420-dependent N5,N10-methylene tetrahydromethanopterin reductase, or other flavin-dependent oxidoreductases.
³A subsequent NCBI search indicates this protein has homology to 17-β-hydroxysterioid dehydrogenase and to Hydratase-dehydrogenase-epimerase. It contains the R-hydratase-like hot dog fold. Other proteins with this fold include fatty acid synthase beta subunit and MaoC dehydratase.
⁴Protein of closest homology with annotated function, based on a BLAST search (E = 10⁻²⁴), is the nitroreductase from Burkholderia dolosa (gi:124901246). Enzymes in this family catalyze the NAD(P)H dependent reduction of flavin or nitro compounds using FMN or FAD as cofactor.

TABLE 3
Mass spectrometry-based identification of proteins captured in the target fishing
study using the CRAA affinity column: analysis of proteins extracted from bands

| | | Molecular | | |
|---|---|---|---|---|
| Accession | | Weight | Percent | |
| number(gi) | | (Da) | Coverage | Score |
| | Annotated Function | | | |
| | (Human) | | | |

A, Human liver proteins in band A (45-60 kDa)

| | | | | |
|---|---|---|---|---|
| 74762137 | Tubulin beta-2A chain* | 49906 | 26 | 148 |
| 21903432 | Isocitrate dehydrogenase [NADP] | 46659 | 24 | 84 |
| | cytoplasmic (CytosolicNADP-isocitrate | | | |
| | dehydrogenase)* | | | |
| 118504 | Aldehyde dehydrogenase, | 56381 | 16 | 36 |
| | mitochondrial precursor (ALDHclass 2)* | | | |
| 55977864 | Tubulin alpha-1A chain (Tubulin B-alpha- | 50135 | 15 | 19 |
| | 1) (Tubulin alpha-3 chain)(Alpha-tubulin | | | |
| | 3)* | | | |

B, Human liver proteins in band B (30-35 kDa)

| | | | | |
|---|---|---|---|---|
| 6648067 | Malate dehydrogenase, | 35,531 | 7 | 3 |
| | mitochondrial precursor* | | | |
| | Annotated function | | | |
| | (Mycobacterium tuberculosis) | | | |

C, Mycobacterium tuberculosis proteins in band C (50-65 kDa)

| | | | | |
|---|---|---|---|---|
| 2829534 | Riboflavin biosynthesis protein ribD | 35,366 | 8 | 4 |
| 1706274 | Bifunctional enzyme cysN/cysC* | 67,839 | 3 | 3 |
| 2497387 | Putative transposase Rv3428c | 45,494 | 2 | 2 |

D, Mycobacterium tuberculosis proteins in band D (30-35 kDa)

| | | | | |
|---|---|---|---|---|
| 81671721 | Possible Oxidoreductase* | 33,220 | 27 | 78 |
| 81668779 | Exopolysaccharide phosphotransferase | 60,268 | 2 | 7 |
| | cpsY | | | |
| 1706274 | Bifunctional enzyme cysN/cysC | 67,839 | 3 | 3 |
| 2829534 | Riboflavin biosynthesis protein ribD* | 35,366 | 8 | 3 |

*Only these proteins have the correct mass for the extracted band(s).

TABLE 4
Mass spectral data from which Table 1 (human) was extracted.

| | | Mr | | Pept. | % | Scan | | |
|---|---|---|---|---|---|---|---|---|
| Annotated function | Accession # (gi) | (g/mol) | (NAD(P)(H)) ? | Cnt. | Coverage | Cnt | Xcorr | Score |
| Malate dehydrogenase | 6648067 | 35,531 | yes | 13 | 51.9 | 181 | 657.3 | 1556.6 |
| Keratin, type II cytoskeletal 1 ( | 1346343 | 66,018 | no | 11 | 23.4 | 122 | 443.9 | 851.7 |
| Fructose-bisphosphate aldolase B | 113611 | 39,473 | no | 7 | 24.3 | 183 | 639.7 | 643.4 |
| Aldehyde dehydrogenase | 118504 | 56,381 | yes | 12 | 27.9 | 94 | 320.5 | 525.5 |
| Glutamate dehydrogenase 1 | 118541 | 61,398 | yes | 10 | 28.5 | 110 | 391.9 | 523.3 |
| Retinal dehydrogenase 1 | 118495 | 54,862 | yes | 8 | 22.4 | 97 | 326.1 | 376.6 |
| Keratin, type I cytoskelatal 9 | 81175178 | 62,129 | no | 9 | 29.9 | 75 | 267.4 | 346.1 |
| 10-formyltetrahydrofolate | 59802911 | 98,829 | yes | 7 | 15.3 | 67 | 234.8 | 252.2 |
| dehydrogenase | | | | | | | | |
| Phosphatidylethanolamine- | 1352726 | 21,057 | no | 4 | 58.4 | 53 | 235.2 | 178.3 |
| binding protein 1 | | | | | | | | |
| Homoglobin subunit beta | 56749856 | 15,998 | no | 3 | 40.0 | 97 | 315.1 | 168.5 |
| Bile salt sulfotransferase | 1711591 | 33,780 | no | 3 | 25.8 | 56 | 216.0 | 153.3 |
| Alcohol dehydrogenase | 113600 | 36,573 | yes | 5 | 28.5 | 62 | 194.5 | 149.4 |
| Heat shock protein HSP 90-alpha | 92090606 | 84,659 | no (ATP-binding) | 6 | 12.9 | 42 | 139.2 | 99.0 |
| Peroxiredoxin-1 | 548453 | 22,110 | yes | 3 | 20.7 | 72 | 209.6 | 92.2 |
| Keratin, type I cytoskeletal 10 | 147744568 | 59,510 | no | 7 | 21.8 | 32 | 129.6 | 83.1 |
| Aldehyde oxidase | 549451 | 147,930 | yes | 5 | 8.9 | 34 | 104.7 | 82.8 |
| Peptidyl-prolyl cis-trans | 51702775 | 18,012 | no | 5 | 35.6 | 35 | 115.2 | 69.0 |
| isomerase A | | | | | | | | |
| Keratin, type II cytoskeletal | 547754 | 65,885 | no | 4 | 12.0 | 27 | 91.7 | 55.3 |
| 2 epidermal | | | | | | | | |
| Isocitrate dehydrogenase | 21903432 | 46,659 | yes | 1 | 24.9 | 19 | 61.8 | 53.1 |
| Pyruvate kinase isozymes R/L | 8247933 | 61,830 | no | 4 | 19.5 | 21 | 68.8 | 41.4 |
| Endoplasmin precursor | 119360 | 92,469 | no | 3 | 5.5 | 29 | 95.1 | 41.4 |
| Alcohol dehydrogenase 1A | 113390 | 39,858 | yes | 3 | 16.4 | 36 | 104.1 | 39.7 |
| | 1352403 | 36,814 | no | 3 | 19.3 | 23 | 80.4 | 35.8 |
| Alpha-aminoadipic | 116241244 | 55,366 | yes | 5 | 16.5 | 22 | 60.1 | 35.0 |
| semialdehyde dehydrogenase | | | | | | | | |
| Hydroxymethylglutaryl-CoA synthase | 1708234 | 56,635 | no | 2 | 14.4 | 16 | 54.3 | 26.6 |
| Arginase- 1 | 12230985 | 34,735 | no | 3 | 17.1 | 17 | 49.4 | 24.5 |
| Serum albumin precursor | 113576 | 69,366 | no | 3 | 6.9 | 18 | 51.0 | 21.4 |
| Delta(3,5)-Delta(2,4)- | 82654933 | 35,816 | no | 2 | 9.5 | 19 | 75.0 | 18.0 |
| dienoyl-CoA isomerase | | | | | | | | |
| 4-trimethylaminobutyraldehyde | 62511242 | 53,802 | yes | 2 | 7.9 | 18 | 63.3 | 16.7 |
| dehydrogenase | | | | | | | | |
| Peroxiredoxin-2 | 2507169 | 21,892 | yes | 4 | 24.5 | 13 | 39.4 | 16.3 |
| NADP-dependent leukotriene | 23503081 | 35,870 | yes | 3 | 17.1 | 19 | 54.7 | 14.8 |
| B4 12-hydroxydehydro | | | | | | | | |
| 10 kDa heat shock protein | 47606335 | 10,932 | no | 2 | 20.0 | 26 | 75.5 | 12.9 |
| Ribonuclease UK114 | 1717975 | 14,494 | no | 1 | 11.8 | 25 | 92.7 | 10.8 |
| Profilin- 1 | 130979 | 15,054 | no | 2 | 33.3 | 9 | 29.8 | 10.1 |
| 2,4-dienoyl-CoA reductase | 3913456 | 36,068 | yes | 1 | 9.9 | 15 | 49.5 | 9.6 |
| Protein disulfide-isomerase precursor | 2507460 | 57,116 | no | 1 | 7.5 | 8 | 22.6 | 9.5 |
| Adenylate kinase isoenzyme 2 | 1708596 | 26,478 | no (ATP-binding) | 2 | 12.2 | 14 | 39.8 | 9.3 |
| Delta-aminolevulinic acid | 122833 | 36,295 | no | 1 | 5.8 | 21 | 71.7 | 9.3 |
| dehydrogenase | | | | | | | | |
| PR domain-containing protein 11 | 25008957 | 57,033 | no | 1 | 2.2 | 28 | 66.1 | 7.8 |
| Quinone oxidereductase | 585013 | 35,206 | yes | 1 | 7.6 | 14 | 40.8 | 7.0 |
| Fumarylacetoacetase | 119778 | 46,374 | no | 1 | 6.9 | 10 | 33.4 | 6.9 |
| Zinc finger CCHC domain- | 116242850 | 185,165 | no | 1 | 1.0 | 21 | 58.4 | 6.7 |
| containing protein 11 | | | | | | | | |
| Actin, cytoplasmic 1 | 46397333 | 41,737 | no (ATP-binding) | 2 | 18.4 | 8 | 25.0 | 6.6 |
| Carbonyl reductase [NADPH] 3 | 6014959 | 30,850 | yes | 1 | 10.5 | 8 | 27.8 | 6.1 |

TABLE 5
Mass spectral data from which Table 2 (M. tb) was extracted.

| | | Mr | | Pept. | % | Scan | | |
|---|---|---|---|---|---|---|---|---|
| Annotated function | Accession # (gi) | (g/mol) | (NAD(P)(H)) ? | Cnt. | Coverage | Cnt | Xcorr | Score |
| POSSIBLE OXIDOREDUCTASE | 81671721 | 33,220 | yes | 3 | 29.7 | 38 | 111.4 | 80.2 |
| Chaperone protein clpB | 54036852 | 92,568 | no (ATP-binding) | 2 | 14.9 | 15 | 36.8 | 31.4 |
| Riboflavin biosynthesis | 2829534 | 35,366 | yes | 2 | 8.3 | 39 | 101.4 | 26.4 |
| protein ribD | | | | | | | | |
| Bifunctional enzyme cysN/cysC | 1706274 | 67,839 | no (ATP-binding, G [symbol] | 1 | 3.4 | 67 | 166.3 | 20.3 |
| Putative uncharacterized protein | 81671959 | 30,296 | unknown | 2 | 30.1 | 12 | 30.6 | 16.6 |
| Putative uncharacterized protein | 81340808 | 38,520 | unkonown | 2 | 15.7 | 14 | 37.5 | 13.4 |
| 27 kDa antigen Clp30B | 61220931 | 27,343 | no | 1 | 7.7 | 27 | 90.9 | 11.6 |
| Glucosamine-fructose-6- | 61224550 | 67,572 | no | 1 | 4.7 | 25 | 50.2 | 10.8 |
| phosphate aminotransferase | | | | | | | | |
| PROBABLE ENOYL-CoA HYDRATASE | 81669965 | 26,630 | no | 1 | 12.1 | 15 | 46.5 | 10.8 |
| ECHA16 | | | | | | | | |
| Hypothetical protein | 81669417 | 33,039 | unknown | 2 | 13.0 | 8 | 23.7 | 6.2 |
| Glutamyl-tRNA synthetase | 61247969 | 53,864 | no (ATP-binding) | 1 | 7.6 | 7 | 18.9 | 6.0 |
| Putative glucanase glgE | 54041254 | 78,640 | no | 2 | 7.7 | 7 | 14.9 | 5.4 |
| Hypothetical protein | 81669395 | 23,172 | unknown | 1 | 9.4 | 19 | 42.8 | 5.0 |
| Probable thiol peroxidase | 54042019 | 16,896 | yes | 1 | 11.0 | 17 | 37.9 | 4.8 |
| 14 kDa antigen | 61217071 | 16,227 | no | 1 | 24.5 | 8 | 16.5 | 3.7 |
| Pyruvate dehydrogenase E1 | 1709445 | 199,216 | yes | 3 | 4.1 | 5 | 9.6 | 3.3 |
| component | | | | | | | | |
| 30S ribosomal protein S4 | 6094181 | 23,476 | no | 1 | 6.5 | 10 | 22.8 | 2.5 |
| Glutamyl-tRNA reductase | 54041345 | 49,361 | yes | 2 | 2.8 | 6 | 11.1 | 2.4 |
| PROBABLE O-ANTIGEN/ | 81817573 | 29,992 | no (ATP-binding) | 2 | 11.0 | 5 | 9.2 | 2.3 |
| LIPOPOLYSACCHARIDE TR [symbol] | | | | | | | | |
| POSSIBLE ENOYL-CoA HYDRATASE | 81671621 | 29,101 | | 1 | 4.0 | 6 | 17.6 | 2.2 |
| ECHA21 | | | | | | | | |
| DNA gyrase subunit B | 158517773 | 74,090 | no (ATP-binding) | 1 | 4.2 | 6 | 11.8 | 2.0 |

[symbol]

indicates data missing or illegible when filed

TABLE 6
Prestwick Chemical Library of Pharmaceuticals

| | | |
|---|---|---|
| Azaguanine-8 | Primaquine diphosphate | Torsemide |
| Allantoin | Progesterone | Halofantrine hydrochloride |
| Acetazolamide | Felodipine | Articaine hydrochloride |
| Metformin hydrochloride | Serotonin hydrochloride | Nomegestrol acetate |
| Atracurium besylate | Cefotiam hydrochloride | Pancuronium bromide |
| Isoflupredone acetate | Benperidol | Molindone hydrochloride |
| Amiloride hydrochloride dihydrate | Cefaclor | Alcuronium chloride |
| Amprolium hydrochloride | Colistin sulfate | Zalcitabine |
| Hydrochlorothiazide | Daunorubicin hydrochloride | Methyldopate hydrochloride |
| Sulfaguanidine | Dosulepin hydrochloride | Levocabastine hydrochloride |
| Meticrane | Ceftazidime pentahydrate | Pyrvinium pamoate |
| Benzonatate | Iobenguane sulfate | Etomidate |
| Hydroflumethiazide | Metixene hydrochloride | Tridihexethyl chloride |
| Sulfacetamide sodic hydrate | Nitrofural | Penbutolol sulfate |
| Heptaminol hydrochloride | Omeprazole | Prednicarbate |
| Sulfathiazole | Propylthiouracil | Sertaconazole nitrate |
| Levodopa | Terconazole | Repaglinide |
| Idoxuridine | Tiaprofenic acid | Piretanide |
| Captopril | Vancomycin hydrochloride | Piperacetazine |
| Minoxidil | Artemisinin | Oxyphenbutazone |
| Sulfaphenazole | Propafenone hydrochloride | Quinethazone |
| Panthenol (D) | Ethamivan | Moricizine hydrochloride |
| Sulfadiazine | Vigabatrin | Iopanoic acid |
| Norethynodrel | Biperiden hydrochloride | Pivmecillinam hydrochloride |
| Thiamphenicol | Cetirizine dihydrochloride | Levopropoxyphene napsylate |
| Cimetidine | Etifenin | Piperidolate hydrochloride |
| Doxylamine succinate | Metaproterenol sulfate, | Trifluridine |
| | orciprenaline sulfate | |
| Ethambutol dihydrochloride | Sisomicin sulfate | Oxprenolol hydrochloride |
| Antipyrine | Resveratrol | Ondansetron Hydrochloride |
| Antipyrine, 4-hydroxy | Bromperidol | Propoxycaine hydrochloride |
| Chloramphenicol | Cyclizine hydrochloride | Oxaprozin |
| Epirizole | Fluoxetine hydrochloride | Phensuximide |
| Diprophylline | Iohexol | Ioxaglic acid |
| Triamterene | Norcyclobenzaprine | Naftifine hydrochloride |
| Dapsone | Pyrazinamide | Meprylcaine hydrochloride |
| Troleandomycin | Trimethadione | Milrinone |
| Pyrimethamine | Lovastatin | Methantheline bromide |
| Hexamethonium dibromide dihydrate | Nystatine | Ticarcillin sodium |
| Diflunisal | Budesonide | Thiethylperazine malate |
| Niclosamide | Imipenem | Mesalamine |
| Procaine hydrochloride | Sulfasalazine | Imidurea |
| Moxisylyte hydrochoride | Thiostrepton | Lansoprazole |
| Betazole hydrochloride | Tiabendazole | Bethanechol chloride |
| Isoxicam | Rifampicin | Cyproterone acetate |
| Naproxen | Ethionamide | (R)-Propranolol hydrochloride |
| Naphazoline hydrochloride | Tenoxicam | Ciprofibrate |
| Ticlopidine hydrochloride | Triflusal | Benzylpenicillin sodium |
| Dicyclomine hydrochloride | Mesoridazine besylate | Chlorambucil |
| Amyleine hydrochloride | Trolox | Methiazole |
| Lidocaine hydrochloride | Pirenperone | (S)-propranolol hydrochloride |
| Trichlorfon | Isoquinoline, 6,7-dimethoxy-1- | (−)-Eseroline fumarate salt |
| | methyl-1,2,3,4-tetrahydro, | |
| | hydrochloride | |
| Carbamazepine | Phenacetin | Leucomisine |
| Triflupromazine hydrochloride | Atovaquone | D-cycloserine |
| Mefenamic acid | Methoxamine hydrochloride | 2-Chloropyrazine |
| Acetohexamide | (R)-(+)-Atenolol | (+,−)-Synephrine |
| Sulpiride | Piracetam | (S)-(−)-Cycloserine |
| Benoxinate hydrochloride | Phenindione | Homosalate |
| Oxethazaine | Thiocolchicoside | Spaglumic acid |
| Pheniramine maleate | Clorsulon | Ranolazine |
| Tolazoline hydrochloride | Ciclopirox ethanolamine | Sulfadoxine |
| Morantel tartrate | Probenecid | Cyclopentolate hydrochloride |
| Homatropine hydrobromide (R,S) | Betahistine mesylate | Estriol |
| Nifedipine | Tobramycin | (−)-Isoproterenol hydrochloride |
| Chlorpromazine hydrochloride | Tetramisole hydrochloride | Nialamide |
| Diphenhydramine hydrochloride | Pregnenolone | Perindopril |
| Minaprine dihydrochloride | Molsidomine | Fexofenadine HCl |
| Miconazole | Chloroquine diphosphate | Clonixin Lysinate |
| Isoxsuprine hydrochloride | Trimetazidine dihydrochloride | Verteporfin |
| Acebutolol hydrochloride | Parthenolide | Meropenem |
| Tolnaftate | Hexetidine | Ramipril |
| Todralazine hydrochloride | Selegiline hydrochloride | Mephenytoin |
| Imipramine hydrochloride | Pentamidine isethionate | Rifabutin |
| Sulindac | Tolazamide | Parbendazole |
| Amitryptiline hydrochloride | Nifuroxazide | Mecamylamine hydrochloride |
| Adiphenine hydrochloride | Dirithromycin | Procarbazine hydrochloride |
| Dibucaine | Gliclazide | Viomycin sulfate |
| Prednisone | DO 897/99 | Saquinavir mesylate |
| Thioridazine hydrochloride | Prenylamine lactate | Ronidazole |
| Diphemanil methylsulfate | Atropine sulfate monohydrate | Dorzolamide hydrochloride |
| Trimethobenzamide hydrochloride | Eserine sulfate, physostigmine | Azaperone |
| | sulfate | |
| Metronidazole | Tetracaine hydrochloride | Cefepime hydrochloride |
| Edrophonium chloride | Mometasone furoate | Clocortolone pivalate |
| Moroxidine hydrochloride | Dacarbazine | Nadifloxacin |
| Baclofen (R,S) | Acetopromazine maleate salt | Carbadox |
| Acyclovir | Lobelanidine hydrochloride | Oxiconazole Nitrate |
| Diazoxide | Papaverine hydrochloride | Acipimox |
| Amidopyrine | Yohimbine hydrochloride | Benazepril HCl |
| Pindolol | Lobeline alpha (−) hydrochoride | Azelastine HCl |
| Khellin | Cilostazol | Celiprolol HCl |
| Zimelidine dihydrochloride | Galanthamine hydrobromide | Cytarabine |
| monohydrate | | |
| Azacyclonol | Diclofenac sodium | Doxofylline |
| Azathioprine | Convolamine hydrochloride | Esmolol hydrochloride |
| Lynestrenol | Xylazine | Itraconazole |
| Guanabenz acetate | Eburnamonine (−) | Liranaftate |
| Disulfiram | Harmaline hydrochloride dihydrate | Mirtazapine |
| Acetylsalicylsalicylic acid | Harmalol hydrochloride dihydrate | Modafinil |
| Mianserine hydrochloride | Harmol hydrochloride monohydrate | Nefazodone HCl |
| Nocodazole | Harmine hydrochloride | Nilvadipine |
| R(−) Apomorphine hydrochloride | Chrysene-1,4-quinone | Oxcarbazepine |
| hemihydrate | | |
| Amoxapine | Demecarium bromide | Rifapentine |
| Cyproheptadine hydrochloride | Quipazine dimaleate salt | Ropinirole HCl |
| Famotidine | Diflorasone Diacetate | Sibutramine HCl |
| Danazol | Harmane hydrochloride | Stanozolol |
| Nicorandil | Methoxy-6-harmalan | Zonisamide |
| Nomifensine maleate | Pyridoxine hydrochloride | Acitretin |
| Dizocilpine maleate | Racecadotril | Rebamipide |
| Naloxone hydrochloride | Folic acid | Diacerein |
| Metolazone | Dimethisoquin hydrochloride | Miglitol |
| Ciprofloxacin hydrochloride | Dipivefrin hydrochloride | Venlafaxine |
| Ampicillin trihydrate | Thiorphan | Irsogladine Maleate |
| Haloperidol | Sulmazole | Acarbose |
| Naltrexone hydrochloride dihydrate | Flunisolide | Carbidopa |
| Chlorpheniramine maleate | N-Acetyl-DL-homocysteine | Aniracetam |
| | Thiolactone | |
| Nalbuphine hydrochloride | Flurandrenolide | Busulfan |
| Picotamide monohydrate | Etanidazole | Docetaxel |
| Triamcinolone | Butirosin disulfate salt | Tibolone |
| Bromocryptine mesylate | Glimepiride | Tizanidine HCl |
| Dehydrocholic acid | Picrotoxinin | Temozolomide |
| Perphenazine | Mepenzolate bromide | Tioconazole |
| Mefloquine hydrochloride | Benfotiamine | granisetron |
| Isoconazole | Halcinonide | ziprasidone Hydrochloride |
| Spironolactone | Lanatoside C | montelukast |
| Pirenzepine dihydrochloride | Benzamil hydrochloride | olmesartan |
| Dexamethasone acetate | Suxibuzone | Oxandrolone |
| Glipizide | 6-Furfurylaminopurine | Thimerosal |
| Loxapine succinate | Avennectin B1a | toltrazuril |
| Hydroxyzine dihydrochloride | Nisoldipine | topotecan |
| Diltiazem hydrochloride | Foliosidine | Toremifene |
| Methotrexate | Dydrogesterone | tranilast |
| Astemizole | Beta-Escin | Tripelennamine hydrochloride |
| Clindamycin hydrochloride | Pempidine tartrate | Clindamycin Phosphate |
| Terfenadine | Nitrarine dihydrochloride | 4-aminosalicylic acid |
| Cefotaxime sodium salt | Estropipate | 5-fluorouracil |
| Tetracycline hydrochloride | Citalopram Hydrobromide | acetylcysteine |
| Verapamil hydrochloride | Promazine hydrochloride | acetylsalicylic acid |
| Dipyridamole | Sulfamerazine | alendronate sodium |
| Chlorhexidine | Ethotoin | alfacalcidol |
| Loperamide hydrochloride | 3-alpha-Hydroxy-5-beta-androstan- | Allopurinol |
| | 17-one | |
| Chlortetracycline hydrochloride | Tetrahydrozoline hydrochloride | amisulpride |
| Tamoxifen citrate | Hexestrol | Amlodipine |
| Nicergoline | Cefmetazole sodium salt | anastrozole |
| Canrenoic acid potassium salt | Trihexyphenidyl-D,L | anethole-trithione |
| | Hydrochloride | |
| Thioproperazine dimesylate | Succinylsulfathiazole | Anthralin |
| Dihydroergotamine tartrate | Famprofazone | argatroban |
| Erythromycin | Bromopride | aripiprazole |
| Didanosine | Methyl benzethonium chloride | atorvastatin |
| Josamycin | Chlorcyclizine hydrochloride | auranofin |
| Paclitaxel | Diphenylpyraline hydrochloride | Azithromycin |
| Ivermectin | Benzethonium chloride | Benztropine mesylate |
| Gallamine triethiodide | Trioxsalen | bicalutamide |
| Neomycin sulfate | Sulfabenzamide | bifonazole |
| Dihydrostreptomycin sulfate | Benzocaine | erlotinib |
| Gentamicin sulfate | Dipyrone | bosentan |
| Isoniazid | Isosorbide dinitrate | bromhexine |
| Pentylenetetrazole | Sulfachloropyridazine | famciclovir |
| Chlorzoxazone | Pramoxine hydrochloride | Butalbital |
| Ornidazole | Finasteride | butenafine |
| Ethosuximide | Fluorometholone | butylscopolammonium (n-) |
| | | bromide |
| Mafenide hydrochloride | Cephalothin sodium salt | fentiazac |
| Riluzole hydrochloride | Cefuroxime sodium salt | caffeine |
| Nitrofurantoin | Althiazide | calcipotriene |
| Hydralazine hydrochloride | Isopyrin hydrochloride | candesartan |
| Phenelzine sulfate | Phenethicillin potassium salt | canrenone |
| Tranexamic acid | Sulfamethoxypyridazine | carprofen |
| Etofylline | Deferoxamine mesylate | carvedilol |
| Tranylcypromine hydrochloride | Mephentermine hemisulfate | Cefdinir |
| Alverine citrate salt | Sulfadimethoxine | gatifloxacin |
| Aceclofenac | Sulfanilamide | gemcitabine |
| Iproniazide phosphate | Balsalazide Sodium | gestrinone |
| Sulfamethoxazole | Sulfaquinoxaline sodium salt | guaiacol |
| Mephenesin | Streptozotocin | gefitinib |
| Phenformin hydrochloride | Metoprolol-(+,−) (+)-tartrate salt | Escitalopram |
| Flutamide | Flumethasone | emedastine |
| Ampyrone | Flecainide acetate | Stavudine |
| Levamisole hydrochloride | Cefazolin sodium salt | mepivacaine hydrochloride |
| Pargyline hydrochloride | Atractyloside potassium salt | Methenamine |
| Methocarbamol | Folinic acid calcium salt | Buspirone hydrochloride |
| Aztreonam | Levonordefrin | ibandronate |
| Cloxacillin sodium salt | Ebselen | ibudilast |
| Catharanthine | Nadide | idebenone |
| Pentolinium bitartrate | Sulfamethizole | imatinib |
| Aminopurine, 6-benzyl | Medrysone | imiquimod |
| Tolbutamide | Flunixin meglumine | ipsapirone |
| Midodrine hydrochloride | Spiramycin | Isosorbide mononitrate |
| Thalidomide | Glycopyrrolate | itopride |
| Oxolinic acid | Cefamandole sodium salt | lacidipine |
| Nimesulide | Monensin sodium salt | lamivudine |
| Hydrastinine hydrochloride | Isoetharine mesylate salt | lapatinib ditosylate |
| Pentoxifylline | Mevalonic-D,L acid lactone | pefloxacine |
| Metaraminol bitartrate | Terazosin hydrochloride | olopatadine |
| Salbutamol | Phenazopyridine hydrochloride | phentermine hydrochloride |
| Prilocaine hydrochloride | Demeclocycline hydrochloride | Phenylbutazone |
| Camptothecine (S,+) | Fenoprofen calcium salt dihydrate | pioglitazone |
| Ranitidine hydrochloride | Piperacillin sodium salt | potassium clavulanate |
| Tiratricol, 3,3′,5-triiodothyroacetic | Diethylstilbestrol | pramipexole |
| acid | | |
| Flufenamic acid | Chlorotrianisene | pranlukast |
| Flumequine | Ribostarmycin sulfate salt | Pranoprofen |
| Tolfenamic acid | Methacholine chloride | Pravastatin |
| Meclofenamic acid sodium salt | Pipenzolate bromide | Prothionamide |
| monohydrate | | |
| Trimethoprim | Butamben | Pyridostigmine iodid |
| Metoclopramide monohydrochloride | Sulfapyridine | Quetiapine |
| Fenbendazole | Meclofenoxate hydrochloride | raclopride |
| Piroxicam | Furaltadone hydrochloride | reboxetine mesylate |
| Pyrantel tartrate | Ethoxyquin | Rimantadine |
| Fenspiride hydrochloride | Tinidazole | rivastigmine |
| Gemfibrozil | Guanadrel sulfate | rofecoxib |
| Mefexamide hydrochloride | Vidarabine | rosiglitazone |
| Tiapride hydrochloride | Sulfameter | rufloxacin |
| Mebendazole | Isopropamide iodide | sarafloxacin |
| Fenbufen | Alclometasone dipropionate | secnidazole |
| Ketoprofen | Leflunomide | sertindole |
| Indapamide | Norgestrel-(−)-D | sildenafil |
| Norfloxacin | Fluocinonide | sparfloxacin |
| Antimycin A | Sulfamethazine sodium salt | sulbactam |
| Xylometazoline hydrochloride | Guaifenesin | sumatriptan succinate |
| Oxymetazoline hydrochloride | Alexidine dihydrochloride | tazobactam |
| Nifenazone | Proadifen hydrochloride | telmisartan |
| Griseofulvin | Zomepirac sodium salt | tenatoprazole |
| Clemizole hydrochloride | Cinoxacin | tulobuterol |
| Tropicamide | Clobetasol propionate | tylosin |
| Nefopam hydrochloride | Podophyllotoxin | vardenafil |
| Phentolamine hydrochloride | Clofibric acid | vatalanib |
| Etodolac | Bendroflumethiazide | vecuronium bromide |
| Scopolamin-N-oxide hydrobromide | Dicumarol | Viloxazine hydrochloride |
| Hyoscyamine (L) | Methimazole | vorinostat |
| Chlorphensin carbamate | Merbromin | Warfarin |
| Metampicillin sodium salt | Hexylcaine hydrochloride | zafirlukast |
| Dilazep dihydrochloride | Drofenine hydrochloride | zileuton |
| Ofloxacin | Cycloheximide | zopiclone |
| Lomefloxacin hydrochloride | (R)-Naproxen sodium salt | zotepine |
| Orphenadrine hydrochloride | Propidium iodide | zaleplon |
| Proglumide | Cloperastine hydrochloride | celecoxib |
| Mexiletine hydrochloride | Eucatropine hydrochloride | chlormadinone acetate |
| Flavoxate hydrochloride | Isocarboxazid | cilnidipine |
| Bufexamac | Lithocholic acid | Clarithromycin |
| Glutethimide, para-amino | Methotrimeprazine maleat salt | clobutinol hydrochloride |
| Dropropizine (R,S) | Dienestrol | clodronate |
| Pinacidil | Pridinol methanesulfonate salt | clofibrate |
| Albendazole | Amrinone | closantel |
| Clonidine hydrochloride | Carbinoxamine maleate salt | desloratadine |
| Bupropion hydrochloride | Methazolamide | Dexfenfluramine hydrochloride |
| Alprenolol hydrochloride | Pyrithyldione | Dibenzepine hydrochloride |
| Chlorothiazide | Spectinomycin dihydrochloride | diclazuril |
| Diphenidol hydrochloride | Piromidic acid | dopamine hydrochloride |
| Norethindrone | Trimipramine maleate salt | doxycycline hydrochloride |
| Nortriptyline hydrochloride | Chloropyramine hydrochloride | Efavirenz |
| Niflumic acid | Furazolidone | Enoxacin |
| Isotretinoin | Dichlorphenamide | Entacapone |
| Retinoic acid | Sulconazole nitrate | Ethinylestradiol |
| Antazoline hydrochloride | Cromolyn disodium salt | Etofenamate |
| Ethacrynic acid | Bucladesine sodium salt | Etoricoxib |
| Praziquantel | Cefsulodin sodium salt | Etretinate |
| Ethisterone | Fosfosal | Exemestane |
| Triprolidine hydrochloride | Suprofen | fleroxacin |
| Doxepin hydrochloride | Catechin-(+,−) hydrate | floxuridine |
| Dyclonine hydrochloride | Nadolol | flubendazol |
| Dimenhydrinate | Moxalactam disodium salt | Fluconazole |
| Disopyramide | Aminophylline | fluocinolone acetonide |
| Clotrimazole | Azlocillin sodium salt | formestane |
| Vinpocetine | Clidinium bromide | formoterol fumarate |
| Clomipramine hydrochloride | Sulfamonomethoxine | Fosinopril |
| Fendiline hydrochloride | Benzthiazide | fulvestrant |
| Vincamine | Trichlormethiazide | levetiracetam |
| Indomethacin | Oxalamine citrate salt | linezolid |
| Cortisone | Propantheline bromide | lofexidine |
| Prednisolone | Dimethadione | loratadine |
| Fenofibrate | Ethaverine hydrochloride | losartan |
| Bumetanide | Butacaine | melengestrol acetate |
| Labetalol hydrochloride | Cefoxitin sodium salt | mevastatin |
| Cinnarizine | Ifosfamide | Misoprostol |
| Methylprednisolone, 6-alpha | Novobiocin sodium salt | Mitotane |
| Quinidine hydrochloride | Tetrahydroxy-1,4-quinone | moxifloxacin |
| monohydrate | monohydrate | |
| Fludrocortisone acetate | Indoprofen | Nalidixic acid sodium salt |
| Fenoterol hydrobromide | Carbenoxolone disodium salt | nicotinamide |
| Homochlorcyclizine dihydrochloride | Iocetamic acid | Norgestimate |
| Diethylcarbamazine citrate | Ganciclovir | Nylidrin |
| Chenodiol | Ethopropazine hydrochloride | olanzapine |
| Perhexiline maleate | Trimeprazine tartrate | opipramol dihydrochloride |
| Oxybutynin chloride | Nafcillin sodium salt monohydrate | oxfendazol |
| Spiperone | Procyclidine hydrochloride | oxibendazol |
| Pyrilamine maleate | Amiprilose hydrochloride | tomoxetine hydrochloride |
| Sulfinpyrazone | Ethynylestradiol 3-methyl ether | Tosufloxacin hydrochloride |
| Dantrolene sodium salt | (−)-Levobunolol hydrochloride | Tramadol hydrochloride |
| Trazodone hydrochloride | Iodixanol | troglitazone |
| Glafenine hydrochloride | Rolitetracycline | Mercaptopurine |
| Pimethixene maleate | Equilin | Amfepramone hydrochloride |
| Pergolide mesylate | Paroxetine Hydrochloride | Hexachlorophene |
| Acemetacin | Liothyronine | Estradiol Valerate |
| Benzydamine hydrochloride | Roxithromycin | Chloroxine |
| Fipexide hydrochloride | Beclomethasone dipropionate | Oxacillin Na |
| Mifepristone | Tolmetin sodium salt dihydrate | Amcinonide |
| Diperodon hydrochloride | (+)-Levobunolol hydrochloride | Penicillamine |
| Lisinopril | Doxazosin mesylate | Rifaximin |
| Lincomycin hydrochloride | Fluvastatin sodium salt | Triclosan |
| Telenzepine dihydrochloride | Methylhydantoin-5-(L) | Racepinephrine HCl |
| Econazole nitrate | Gabapentin | cyclophosphamide |
| Bupivacaine hydrochloride | Raloxifene hydrochloride | Valproic acid |
| Clemastine fumarate | Etidronic acid. disodium salt | Fludarabine |
| Oxytetracycline dihydrate | Methylhydantoin-5-(D) | Cladribine |
| Pimozide | Simvastatin | Cortisol acetate |
| Amodiaquin dihydrochloride | Azacytidine-5 | Mesna |
| dihydrate | | |
| Mebeverine hydrochloride | Paromomycin sulfate | Penciclovir |
| Ifenprodil tartrate | Acetaminophen | amifostine |
| Flunarizine dihydrochloride | Phthalylsulfathiazole | Nalmefene |
| Trifluoperazine dihydrochloride | Luteolin | Pentobarbital |
| Enalapril maleate | Iopamidol | Lamotrigine |
| Minocycline hydrochloride | Iopromide | Topiramate |
| Glibenclamide | Theophylline monohydrate | Irinotecan Hydrochloride |
| Guanethidine sulfate | Theobromine | Rabeprazole |
| Quinacrine dihydrochloride dihydrate | Reserpine | Tiludronate disodium |
| Clofilium tosylate | Scopolamine hydrochloride | Ambrisentan |
| Fluphenazine dihydrochloride | Ioversol | Torsemide |
| Streptomycin sulfate | Carbachol | Halofantrine hydrochloride |
| Alfuzosin hydrochloride | Niacin | Articaine hydrochloride |
| Chlorpropamide | Bemegride | Nomegestrol acetate |
| Phenylpropanolamine hydrochloride | Digoxigenin | Pancuronium bromide |
| Ascorbic acid | Meglumine | Molindone hydrochloride |
| Methyldopa (L,−) | Cantharidin | Alcuronium chloride |
| Cefoperazone dihydrate | Clioquinol | Zalcitabine |
| Zoxazolamine | Oxybenzone | Methyldopate hydrochloride |
| Tacrine hydrochloride hydrate | Promethazine hydrochloride | Levocabastine hydrochloride |
| Bisoprolol fumarate | Felbinac | Pyrvinium pamoate |
| Tremorine dihydrochloride | Butylparaben | Etomidate |
| Practolol | Aminohippuric acid | Tridihexethyl chloride |
| Zidovudine, AZT | N-Acetyl-L-leucine | Penbutolol sulfate |
| Sulfisoxazole | Pipemidic acid | Prednicarbate |
| Zaprinast | Dioxybenzone | Sertaconazole nitrate |
| Chlormezanone | Adrenosterone | Repaglinide |
| Procainamide hydrochloride | Methylatropine nitrate | Piretanide |
| N6-methyladenosine | Hymecromone | Piperacetazine |
| Guanfacine hydrochloride | Caffeic acid | Oxyphenbutazone |
| Domperidone | Diloxanide furoate | Quinethazone |
| Furosemide | Metyrapone | Moricizine hydrochloride |
| Methapyrilene hydrochloride | Urapidil hydrochloride | Iopanoic acid |
| Desipramine hydrochloride | Fluspirilen | Pivmecillinam hydrochloride |
| Clorgyline hydrochloride | S-(+)-ibuprofen | Levopropoxyphene napsylate |
| Clenbuterol hydrochloride | Ethynodiol diacetate | Piperidolate hydrochloride |
| Maprotiline hydrochloride | Nabumetone | Trifluridine |
| Thioguanosine | Nisoxetine hydrochloride | Oxprenolol hydrochloride |
| Chlorprothixene hydrochloride | (+)-Isoproterenol (+)-bitartrate salt | Ondansetron Hydrochloride |
| Ritodrine hydrochloride | Monobenzone | Propoxycaine hydrochloride |
| Clozapine | 2-Aminobenzenesulfonamide | Oxaprozin |
| Chlorthalidone | Estrone | Phensuximide |
| Dobutamine hydrochloride | Lorglumide sodium salt | Ioxaglic acid |
| Moclobemide | Nitrendipine | Naftifine hydrochloride |
| Clopamide | Flurbiprofen | Meprylcaine hydrochloride |
| Hycanthone | Nimodipine | Milrinone |
| Adenosine 5′-monophosphate | Bacitracin | Methantheline bromide |
| monohydrate | | |
| Amoxicillin | L(−)-vesamicol hydrochloride | Ticarcillin sodium |
| Cephalexin monohydrate | Nizatidine | Thiethylperazine malate |
| Dextromethorphan hydrobromide | Thioperamide maleate | Mesalamine |
| monohydrate | | |
| Droperidol | Xamoterol hemifumarate | Imidurea |
| Bambuterol hydrochloride | Rolipram | Lansoprazole |
| Betamethasone | Thonzonium bromide | Bethanechol chloride |
| Colchicine | Idazoxan hydrochloride | Cyproterone acetate |
| Metergoline | Quinapril HCl | (R)-Propranolol hydrochloride |
| Brinzolamide | Nilutamide | Ciprofibrate |
| Ambroxol hydrochloride | Ketorolac tromethamine | Benzylpenicillin sodium |
| Benfluorex hydrochloride | Protriptyline hydrochloride | Chlorambucil |
| Bepridil hydrochloride | Propofol | Methiazole |
| Meloxicam | S(−)Eticlopride hydrochloride | (S)-propranolol hydrochloride |
| Benzbromarone | Primidone | (−)-Eseroline fumarate salt |
| Ketotifen fumarate | Flucytosine | Leucomisine |
| Debrisoquin sulfate | (−)-MK 801 hydrogen maleate | D-cycloserine |
| Amethopterin (R,S) | Bephenium hydroxynaphthoate | 2-Chloropyrazine |
| Methylergometrine maleate | Dehydroisoandosterone 3-acetate | (+,−)-Synephrine |
| Methiothepin maleate | Benserazide hydrochloride | (S)-(−)-Cycloserine |
| Clofazimine | Iodipamide | Homosalate |
| Nafronyl oxalate | Pentetic acid | Spaglumic acid |
| Bezafibrate | Bretylium tosylate | Ranolazine |
| Clebopride maleate | Pralidoxime chloride | Sulfadoxine |
| Lidoflazine | Phenoxybenzamine hydrochloride | Cyclopentolate hydrochloride |
| Betaxolol hydrochloride | Salmeterol | Estriol |
| Nicardipine hydrochloride | Altretamine | (−)-Isoproterenol hydrochloride |
| Probucol | Prazosin hydrochloride | Nialamide |
| Mitoxantrone dihydrochloride | Timolol maleate salt | Perindopril |
| GBR 12909 dihydrochloride | (+,−)-Octopamine hydrochloride | Fexofenadine HCl |
| Carbetapentane citrate | Crotamiton | Clonixin Lysinate |
| Dequalinium dichloride | (S)-(−)-Atenolol | Verteporfin |
| Ketoconazole | Tyloxapol | Meropenem |
| Fusidic acid sodium salt | Florfenicol | Ramipril |
| Terbutaline hemisulfate | Megestrol acetate | Mephenytoin |
| Ketanserin tartrate hydrate | Deoxycorticosterone | Rifabutin |
| Hemicholinium bromide | Urosiol | Parbendazole |
| Kanamycin A sulfate | Proparacaine hydrochloride | Mecamylamine hydrochloride |
| Amikacin hydrate | Aminocaproic acid | Procarbazine hydrochloride |
| Etoposide | Denatonium benzoate | Viomycin sulfate |
| Clomiphene citrate (Z,E) | Enilconazole | Saquinavir mesylate |
| Oxantel pamoate | Methacycline hydrochloride | Ronidazole |
| Prochlorperazine dimaleate | Sotalol hydrochloride | Dorzolamide hydrochloride |
| Hesperidin | Decamethonium bromide | Azaperone |
| Testosterone propionate | 3-Acetamidocoumarin | Cefepime hydrochloride |
| Arecoline hydrobromide | Roxarsone | Clocortolone pivalate |
| Thyroxine (L) | Remoxipride Hydrochloride | Nadifloxacin |
| Pepstatin A | THIP Hydrochloride | Carbadox |
| SR-95639A | Pirlindole mesylate | Oxiconazole Nitrate |
| Adamantamine fumarate | Pronethalol hydrochloride | Acipimox |
| Butoconazole nitrate | Naftopidil dihydrochloride | Benazepril HCl |
| Amiodarone hydrochloride | Tracazolate hydrochloride | Azelastine HCl |
| Amphotericin B | Zardaverine | Celiprolol HCl |
| Androsterone | Memantine Hydrochloride | Cytarabine |
| Carbarsone | Ozagrel hydrochloride | Doxofylline |
| Bacampicillin hydrochloride | Piribedil hydrochloride | Esmolol hydrochloride |
| Biotin | Nitrocaramiphen hydrochloride | Itraconazole |
| Bisacodyl | Nandrolone | Liranaftate |
| Suloctidil | Dimaprit dihydrochloride | Mirtazapine |
| Carisoprodol | Proscillaridin A | Modafinil |
| Cephalosporanic acid, 7-amino | Gliquidone | Nefazodone HCl |
| Chicago sky blue 6B | Pizotifen malate | Nilvadipine |
| Buflomedil hydrochloride | Ribavirin | Oxcarbazepine |
| Roxatidine Acetate HCl | Cyclopenthiazide | Rifapentine |
| Cholecalciferol | Fluvoxamine maleate | Ropinirole HCl |
| Cisapride | Fluticasone propionate | Sibutramine HCl |
| Corticosterone | Zuclopenthixol hydrochloride | Stanozolol |
| Cyanocobalamin | Proguanil hydrochloride | Zonisamide |
| Cefadroxil | Lymecycline | Acitretin |
| Cyclosporin A | Alfadolone acetate | Rebamipide |
| Digitoxigenin | Alfaxalone | Diacerein |
| Digoxin | Azapropazone | Miglitol |
| Doxorubicin hydrochloride | Meptazinol hydrochloride | Venlafaxine |
| Carbimazole | Apramycin | Irsogladine Maleate |
| Epiandrosterone | Epitiostanol | Acarbose |
| Estradiol-17 beta | Fursultiamine Hydrochloride | Carbidopa |
| Gabazine | Gabexate mesilate | Aniracetam |
| Cyclobenzaprine hydrochloride | Pivampicillin | Busulfan |
| Carteolol hydrochloride | Talampicillin hydrochloride | Docetaxel |
| Hydrocortisone base | Flucloxacillin sodium | Tibolone |
| Hydroxytacrine maleate (R,S) | Trapidil | Tizanidine HCl |
| Pilocarpine nitrate | Deptropine citrate | Temozolomide |
| Dicloxacillin sodium salt | Sertraline | Tioconazole |
| Alizapride HCl | Ethamsylate | granisetron |
| Mebhydroline 1,5- | Moxonidine | ziprasidone Hydrochloride |
| naphtalenedisulfonate | | |
| Meclocycline sulfosalicylate | Etilefrine hydrochloride | montelukast |
| Meclozine dihydrochloride | Alprostadil | olmesartan |
| Melatonin | Tribenoside | Oxandrolone |
| Dinoprost trometamol | Rimexolone | Thimerosal |
| Tropisetron HCl | Isradipine | toltrazuril |
| Cefixime | Tiletamine hydrochloride | topotecan |
| Metrizamide | Isometheptene mucate | Toremifene |
| Neostigmine bromide | Nifurtimox | tranilast |
| Niridazole | Letrozole | Tripelennamine hydrochloride |
| Ceforanide | Arbutin | Clindamycin Phosphate |
| Cefotetan | Tocainide hydrochloride | 4-aminosalicylic acid |
| Brompheniramine maleate | Benzathine benzylpenicillin | 5-fluorouracil |
| Azaguanine-8 | Risperidone | acetylcysteine |

TABLE 7
Microsource: The Spectrum Collection of Pharmaceuticals

| | | |
|---|---|---|
| MAFENIDE HYDROCHLORIDE | CYPROTERONE ACETATE | BENDROFLUMETHIAZIDE |
| MAPROTILINE | CYTARABINE | BEPRIDIL HYDROCHLORIDE |
| HYDROCHLORIDE | | |
| MECAMYLAMINE | DACARBAZINE | BROMHEXINE |
| HYDROCHLORIDE | | HYDROCHLORIDE |
| MECHLORETHAMINE | DANAZOL | CARMUSTINE |
| MECLIZINE HYDROCHLORIDE | DAPSONE | CEFTRIAXONE SODIUM |
| | | TRIHYDRATE |
| MECLOFENAMATE SODIUM | DAUNORUBICIN | TRIMIPRAMINE MALEATE |
| MEDRYSONE | SODIUM DEHYDROCHOLATE | TRIFLUPROMAZINE |
| | | HYDROCHLORIDE |
| MEGESTROL ACETATE | DEMECLOCYCLINE | TRAZODONE |
| | HYDROCHLORIDE | HYDROCHLORIDE |
| MELPHALAN | DESIPRAMINE | MENTHOL(−) |
| | HYDROCHLORIDE | |
| MESTRANOL | DEXAMETHASONE | THONZYLAMINE |
| | | HYDROCHLORIDE |
| METAPROTERENOL | DEXAMETHASONE ACETATE | THIAMPHENICOL |
| METHACHOLINE CHLORIDE | DEFEROXAMINE MESYLATE | TENOXICAM |
| METHIMAZOLE | DEXAMETHASONE SODIUM | CHLOROXINE |
| | PHOSPHATE | |
| METHOCARBAMOL | DEXTROMETHORPHAN | CHLORPROTHIXENE |
| | HYDROBROMIDE | HYDROCHLORIDE |
| METHOTREXATE(+/−) | DIBENZOTHIOPHENE | CINNARAZINE |
| METHOXAMINE | DIBUCAINE | DANTROLENE SODIUM |
| HYDROCHLORIDE | HYDROCHLORIDE | |
| METHYLDOPA | DICLOFENAC SODIUM | BETAMETHASONE 17,21- |
| | | DIPROPIONATE |
| METHYLPREDNISOLONE | DICLOXACILLIN SODIUM | DOBUTAMINE |
| | | HYDROCHLORIDE |
| METOCLOPRAMIDE | DICUMAROL | EDOXUDINE |
| HYDROCHLORIDE | | |
| METOPROLOL TARTRATE | DICYCLOMINE | ENOXACIN |
| | HYDROCHLORIDE | |
| METRONIDAZOLE | DIENESTROL | ETHISTERONE |
| MINOCYCLINE | DIETHYLCARBAMAZINE | PARAROSANILINE PAMOATE |
| HYDROCHLORIDE | CITRATE | |
| MINOXIDIL | DIETHYLSTILBESTROL | PERHEXILINE MALEATE |
| MOXALACTAM DISODIUM | DIFLUNISAL | PAROMOMYCIN SULFATE |
| NADIDE | DIGITOXIN | METHAPYRILENE |
| | | HYDROCHLORIDE |
| NAFCILLIN SODIUM | DIGOXIN | BETA-PROPIOLACTONE |
| NALOXONE HYDROCHLORIDE | DIHYDROERGOTAMINE | HALCINONIDE |
| | MESYLATE | |
| NAPHAZOLINE | DIHYDROSTREPTOMYCIN | HYCANTHONE |
| HYDROCHLORIDE | SULFATE | |
| NAPROXEN(+) | DIMENHYDRINATE | PYRIDOSTIGMINE BROMIDE |
| NEOSTIGMINE BROMIDE | DIMETHADIONE | ISOXICAM |
| NIACIN | DIOXYBENZONE | LABETALOL |
| | | HYDROCHLORIDE |
| NIFEDIPINE | DIPHENHYDRAMINE | LEVAMISOLE |
| | HYDROCHLORIDE | HYDROCHLORIDE |
| NITROFURANTOIN | DIPHENYLPYRALINE | MEPHENTERMINE SULFATE |
| | HYDROCHLORIDE | |
| OXYBUTYNIN CHLORIDE | DIPYRIDAMOLE | METARAMINOL BITARTRATE |
| NOREPINEPHRINE | PYRITHIONE ZINC | METHAZOLAMIDE |
| NORETHINDRONE | DISOPYRAMIDE PHOSPHATE | METHYLBENZETHONIUM |
| | | CHLORIDE |
| NORETHYNODREL | DISULFIRAM | METHYLPREDNISOLONE |
| | | SODIUM SUCCINATE |
| NORFLOXACIN | DOPAMINE | AMSACRINE |
| | HYDROCHLORIDE | |
| NORGESTREL | DOXEPIN HYDROCHLORIDE | MIDODRINE |
| | | HYDROCHLORIDE |
| NORTRIPTYLINE | DOXYCYCLINE | NADOLOL |
| | HYDROCHLORIDE | |
| NOSCAPINE HYDROCHLORIDE | DOXYLAMINE SUCCINATE | NALTREXONE |
| | | HYDROCHLORIDE |
| NOVOBIOCIN SODIUM | DYCLONINE | CYCLOTHIAZIDE |
| | HYDROCHLORIDE | |
| NYLIDRIN HYDROCHLORIDE | DYPHYLLINE | NICLOSAMIDE |
| NYSTATIN | TRISODIUM | NOMIFENSINE MALEATE |
| | ETHYLENEDIAMINE | |
| | TETRACETATE | |
| ORPHENADRINE CITRATE | EMETINE | PERGOLIDE MESYLATE |
| OXACILLIN SODIUM | ADRENALINE BITARTRATE | PRILOCAINE |
| | | HYDROCHLORIDE |
| OXYBENZONE | EQUILIN | HYDROCORTISONE |
| | | BUTYRATE |
| OXYMETAZOLINE | ERGOCALCIFEROL | ROXITHROMYCIN |
| HYDROCHLORIDE | | |
| OXYPHENBUTAZONE | ERGONOVINE MALEATE | MITOXANTHRONE |
| | | HYDROCHLORIDE |
| OXYTETRACYCLINE | ERYTHROMYCIN | OXETHAZAINE |
| | ETHYLSUCCINATE | |
| PAPAVERINE | ESTRADIOL | DIPYRONE |
| HYDROCHLORIDE | | |
| PARACHLOROPHENOL | ESTRADIOL CYPIONATE | SULFANILATE ZINC |
| PARGYLINE HYDROCHLORIDE | ESTRADIOL VALERATE | URETHANE |
| PENICILLAMINE | ESTRIOL | THIRAM |
| PHENACEMIDE | ESTRONE | THIOTEPA |
| PHENAZOPYRIDINE | ETHACRYNIC ACID | TETROQUINONE |
| HYDROCHLORIDE | | |
| PHENELZINE SULFATE | ETHAMBUTOL | SULFANITRAN |
| | HYDROCHLORIDE | |
| PHENINDIONE | ETHINYL ESTRADIOL | OXIBENDAZOLE |
| PHENIRAMINE MALEATE | ETHIONAMIDE | PIPOBROMAN |
| PHENYLBUTAZONE | ETHOPROPAZINE | ETANIDAZOLE |
| | HYDROCHLORIDE | |
| PHENYTOIN SODIUM | EUCATROPINE | NAFRONYL OXALATE |
| | HYDROCHLORIDE | |
| FENOFIBRATE | EUGENOL | QUIPAZINE MALEATE |
| FENOPROFEN | FLUDROCORTISONE | RITANSERIN |
| | ACETATE | |
| FLUFENAMIC ACID | FLUMETHAZONE PIVALATE | SEMUSTINE |
| FENBENDAZOLE | FLUOCINOLONE ACETONIDE | SPIRAMYCIN |
| FENSPIRIDE HYDROCHLORIDE | FLUOCINONIDE | CLOFIBRATE |
| MEFENAMIC ACID | FLUOROMETHOLONE | RESORCINOL MONOACETATE |
| METHACYCLINE | FLUOROURACIL | NIMODIPINE |
| HYDROCHLORIDE | | |
| MEFEXAMIDE | FLURBIPROFEN | ACYCLOVIR |
| PROBUCOL | FURAZOLIDONE | RETINYL PALMITATE |
| PUROMYCIN | FUROSEMIDE | THALIDOMIDE |
| HYDROCHLORIDE | | |
| MEBENDAZOLE | FUSIDIC ACID | NITRENDIPINE |
| NALBUPHINE | GALLAMINE TRIETHIODIDE | BENZALKONIUM CHLORIDE |
| HYDROCHLORIDE | | |
| PROGLUMIDE | GEMFIBROZIL | CIPROFLOXACIN |
| MINAPRINE HYDROCHLORIDE | GENTAMICIN SULFATE | CELECOXIB |
| MEMANTINE | GENTIAN VIOLET | AZITHROMYCIN |
| HYDROCHLORIDE | | |
| ATENOLOL | GLUCOSAMINE | ANETHOLE |
| | HYDROCHLORIDE | |
| CARBETAPENTANE CITRATE | GRAMICIDIN | TERFENADINE |
| PIMOZIDE | GUAIFENESIN | CLOPIDOGREL SULFATE |
| NICARDIPINE | GUANABENZ ACETATE | LORATADINE |
| HYDROCHLORIDE | | |
| NEFOPAM | GUANETHIDINE SULFATE | SELAMECTIN |
| PIRENZEPINE | HALAZONE | NAPROXOL |
| HYDROCHLORIDE | | |
| PRAMOXINE | HALOPERIDOL | COLFORSIN |
| HYDROCHLORIDE | | |
| MEPHENESIN | HETACILLIN POTASSIUM | ISOSORBIDE MONONITRATE |
| SULFACHLORPYRIDAZINE | HEXACHLOROPHENE | AMCINONIDE |
| SULFADIMETHOXINE | HEXYLRESORCINOL | BUPIVACAINE |
| | | HYDROCHLORIDE |
| SULFAGUANIDINE | HISTAMINE | ALBENDAZOLE |
| | DIHYDROCHLORIDE | |
| SULFAMONOMETHOXINE | HOMATROPINE BROMIDE | PACLITAXEL |
| SULCONAZOLE NITRATE | HOMATROPINE | BUTACAINE |
| | METHYLBROMIDE | |
| RITODRINE HYDROCHLORIDE | HYDRALAZINE | CLOBETASOL PROPIONATE |
| | HYDROCHLORIDE | |
| SULPIRIDE | HYDROCHLOROTHIAZIDE | IOPANIC ACID |
| RANITIDINE | HYDROCORTISONE ACETATE | KETOROLAC |
| | | TROMETHAMINE |
| SULOCTIDIL | HYDROCORTISONE | LANSOPRAZOLE |
| | HEMISUCCINATE | |
| RONIDAZOLE | HYDROCORTISONE | MEXILETINE |
| | PHOSPHATE | HYDROCHLORIDE |
| | TRIETHYLAMINE | |
| SULFAMETER | HYDROFLUMETHIAZIDE | MORANTEL CITRATE |
| SULFAMETHOXYPYRIDAZINE | HYDROXYPROGESTERONE | PERPHENAZINE |
| | CAPROATE | |
| SUPROFEN | HYDROXYUREA | RIBAVIRIN |
| SACCHARIN | HYDROXYZINE PAMOATE | TACROLIMUS |
| ACETANILIDE | HYOSCYAMINE | BROMPHENIRAMINE |
| | | MALEATE |
| FLURANDRENOLIDE | IBUPROFEN | SIROLIMUS |
| ESTRADIOL ACETATE | IMIPRAMINE | PAROXETINE |
| | HYDROCHLORIDE | HYDROCHLORIDE |
| ECONAZOLE NITRATE | INDAPAMIDE | ETHYLNOREPINEPHRINE |
| | | HYDROCHLORIDE |
| FLUNISOLIDE | INDOMETHACIN | ALAPROCLATE |
| FLUMETHASONE | INDOPROFEN | ACETRIAZOIC ACID |
| XYLAZINE | INOSITOL | VENLAFAXINE |
| TOLAZAMIDE | IODOQUINOL | CITALOPRAM |
| GALANTHAMINE | IPRATROPIUM BROMIDE | FLUOXETINE |
| HYDROBROMIDE | | |
| LANATOSIDE C | ISONIAZID | BUPROPION |
| ENALAPRIL MALEATE | ISOPROPAMIDE IODIDE | CEFUROXIME AXETIL |
| KETOPROFEN | ISOPROTERENOL | FEXOFENADINE |
| | HYDROCHLORIDE | HYDROCHLORIDE |
| LISINOPRIL | ISOSORBIDE DINITRATE | TRIFLURIDINE |
| BUMETANIDE | ISOXSUPRINE | PIRENPERONE |
| | HYDROCHLORIDE | |
| CARBENOXOLONE SODIUM | KANAMYCIN A SULFATE | AVOBENZONE |
| FOLIC ACID | KETOCONAZOLE | ATOVAQUONE |
| PHTHALYLSULFATHIAZOLE | LACTULOSE | TRIMETOZINE |
| SUCCINYLSULFATHIAZOLE | LEUCOVORIN CALCIUM | ZOXAZOLAMINE |
| TRANEXAMIC ACID | LEVONORDEFRIN | CYSTEAMINE |
| | | HYDROCHLORIDE |
| CEPHALEXIN | LINCOMYCIN | ROFECOXIB |
| | HYDROCHLORIDE | |
| OXOLINIC ACID | MEDROXYPROGESTERONE | SIMVASTATIN |
| | ACETATE | |
| CEFOXITIN SODIUM | MEPENZOLATE BROMIDE | OXCARBAZEPINE |
| SURAMIN | MERCAPTOPURINE | MELOXICAM SODIUM |
| CEFUROXIME SODIUM | METHENAMINE | CARVEDILOL |
| VIGABATRIN | METHICILLIN SODIUM | IRBESARTAN |
| LOMEFLOXACIN | METHOXSALEN | LEVOFLOXACIN |
| HYDROCHLORIDE | | |
| CEFAMANDOLE SODIUM | METHYLERGONOVINE | LITHIUM CITRATE |
| | MALEATE | |
| CEFMETAZOLE SODIUM | METHYLTHIOURACIL | GATIFLOXACIN |
| CEFOPERAZONE SODIUM | MICONAZOLE NITRATE | MIGLITOL |
| OFLOXACIN | NEOMYCIN SULFATE | ORLISTAT |
| BEZAFIBRATE | NITROFURAZONE | MOXIFLOXACIN |
| | | HYDROCHLORIDE |
| CETIRIZINE HYDROCHLORIDE | NITROMIDE | PIOGLITAZONE |
| | | HYDROCHLORIDE |
| PHENYLETHYL ALCOHOL | NORETHINDRONE ACETATE | DONEPEZIL HYDROCHLORIDE |
| MECLOCYCLINE | OXIDOPAMINE | FLUVASTATIN |
| SULFOSALICYLATE | HYDROCHLORIDE | |
| RIBOFLAVIN | OXYQUINOLINE | PIZOTYLINE MALATE |
| | HEMISULFATE | |
| ACEBUTOLOL | PENICILLIN G POTASSIUM | EXEMESTANE |
| HYDROCHLORIDE | | |
| ASPARTAME | PENICILLIN V POTASSIUM | TILMICOSIN |
| VARDENAFIL | PHENOLPHTHALEIN | FLUNIXIN MEGLUMINE |
| HYDROCHLORIDE | | |
| FLUORESCEIN | PHENYLEPHRINE | CLORSULON |
| | HYDROCHLORIDE | |
| NIACINAMIDE | PHENYLPROPANOLAMINE | ESTROPIPATE |
| | HYDROCHLORIDE | |
| PROPRANOLOL | PHYSOSTIGMINE | CLAVULANATE LITHIUM |
| HYDROCHLORIDE (+/−) | SALICYLATE | |
| METHSCOPOLAMINE | PILOCARPINE NITRATE | ALCLOMETAZONE |
| BROMIDE | | DIPROPIONATE |
| EDROPHONIUM CHLORIDE | PINDOLOL | ALENDRONATE SODIUM |
| THIOPENTAL SODIUM | PIPERACILLIN SODIUM | ACARBOSE |
| PENTOBARBITAL | PIPERAZINE | ROPINIROLE |
| PHENFORMIN | PIROXICAM | QUETIAPINE |
| HYDROCHLORIDE | | |
| PENFLURIDOL | POLYMYXIN B SULFATE | RIZATRIPTAN BENZOATE |
| PHTHALYSULFATHIAZOLE | PRAZIQUANTEL | FAMCICLOVIR |
| VINCRISTINE SULFATE | PRAZOSIN HYDROCHLORIDE | AMLODIPINE BESYLATE |
| OMEPRAZOLE | PREDNISOLONE | EZETIMIBE |
| ZOLMITRIPTAN | PREDNISOLONE ACETATE | OLMESARTAN MEDOXOMIL |
| DEBRISOQUIN SULFATE | PREDNISONE | CEFTIBUTEN |
| SULFADOXINE | PRIMAQUINE DIPHOSPHATE | CEFDINIR |
| FINASTERIDE | PRIMIDONE | SIBUTRAMINE |
| | | HYDROCHLORIDE |
| PENTETIC ACID | PROBENECID | PERINDOPRIL ERBUMINE |
| PROSCILLARIDIN | PROCAINAMIDE | ROSUVASTATIN CALCIUM |
| | HYDROCHLORIDE | |
| JOSAMYCIN | PROCAINE HYDROCHLORIDE | RAMIPRIL |
| REPAGLINIDE | PROCHLORPERAZINE | ESCITALOPRAM OXALATE |
| | EDISYLATE | |
| CROTAMITON | PROCYCLIDINE | DERACOXIB |
| | HYDROCHLORIDE | |
| CEFPROZIL | PROMAZINE | CILOSTAZOL |
| | HYDROCHLORIDE | |
| METHYLDOPATE | PROPANTHELINE BROMIDE | CITICOLINE |
| HYDROCHLORIDE | | |
| SULFAQUINOXALINE SODIUM | DEXPROPRANOLOL | APRAMYCIN |
| | HYDROCHLORIDE | |
| POTASSIUM p- | PROPYLTHIOURACIL | SERTRALINE |
| AMINOBENZOATE | | HYDROCHLORIDE |
| BETAMETHASONE VALERATE | PSEUDOEPHEDRINE | ALFLUZOSIN |
| | HYDROCHLORIDE | |
| ERYTHROMYCIN | PYRANTEL PAMOATE | TELITHROMYCIN |
| PROMETHAZINE | PYRAZINAMIDE | OXAPROZIN |
| HYDROCHLORIDE | | |
| SCOPOLAMINE | PYRILAMINE MALEATE | OXFENDAZOLE |
| HYDROBROMIDE | | |
| THEOPHYLLINE | PYRIMETHAMINE | AMITRAZ |
| TOLNAFTATE | PYRVINIUM PAMOATE | PEFLOXACINE MESYLATE |
| TRIMETHOBENZAMIDE | QUINACRINE | CHLOROPHYLLIDE Cu |
| HYDROCHLORIDE | HYDROCHLORIDE | COMPLEX Na SALT |
| VINBLASTINE SULFATE | QUINIDINE GLUCONATE | BIFONAZOLE |
| CLEBOPRIDE MALEATE | QUININE SULFATE | TYLOSIN TARTRATE |
| PIRACETAM | RACEPHEDRINE | SARAFLOXACIN |
| | HYDROCHLORIDE | HYDROCHLORIDE |
| GLUCONOLACTONE | RESERPINE | CLOPIDOL |
| AZLOCILLIN SODIUM | RESORCINOL | CHLORMADINONE ACETATE |
| CHOLINE CHLORIDE | RIFAMPIN | OXICONAZOLE NITRATE |
| ATORVASTATIN CALCIUM | ROXARSONE | AZAPERONE |
| OXYPHENCYCLIMINE | SALICYL ALCOHOL | TRANILAST |
| HYDROCHLORIDE | | |
| PROPAFENONE | SALICYLAMIDE | AZELASTINE |
| HYDROCHLORIDE | | HYDROCHLORIDE |
| FLUCONAZOLE | SODIUM SALICYLATE | KETANSERIN TARTRATE |
| LOVASTATIN | SISOMICIN SULFATE | FIPRONIL |
| ATROPINE OXIDE | SPECTINOMYCIN | DECOQUINATE |
| | HYDROCHLORIDE | |
| SENNOSIDE A | SPIRONOLACTONE | CEFDITORIN PIVOXIL |
| TENIPOSIDE | STREPTOMYCIN SULFATE | VALACYCLOVIR |
| | | HYDROCHLORIDE |
| TANNIC ACID | STREPTOZOSIN | DULOXETINE |
| | | HYDROCHLORIDE |
| CARPROFEN | SULFABENZAMIDE | NISOLDIPINE |
| HYDROXYCHLOROQUINE | SULFACETAMIDE | MONTELUKAST SODIUM |
| SULFATE | | |
| DIRITHROMYCIN | SULFADIAZINE | BENURESTAT |
| MEPIVACAINE | SULFAMERAZINE | BENZOXIQUINE |
| HYDROCHLORIDE | | |
| NILUTAMIDE | SULFAMETHAZINE | BISMUTH SUBSALICYLATE |
| AMINOLEVULINIC ACID | SULFAMETHIZOLE | BENZOYLPAS |
| HYDROCHLORIDE | | |
| PARAMETHADIONE | SULFAMETHOXAZOLE | BROMINDIONE |
| METAXALONE | SULFAPYRIDINE | CAPOBENIC ACID |
| CHLOROGUANIDE | SULFASALAZINE | ACETOHEXAMIDE |
| HYDROCHLORIDE | | |
| CLARITHROMYCIN | SULFATHIAZOLE | ETHOXZOLAMIDE |
| HYDROQUINONE | SULFINPYRAZONE | FLUCYTOSINE |
| NATEGLINIDE | SULFISOXAZOLE | FOMEPIZOLE |
| | | HYDROCHLORIDE |
| CANDESARTAN CILEXTIL | SULINDAC | GLIPIZIDE |
| ROSIGLITAZONE | TAMOXIFEN CITRATE | GUANFACINE |
| LOSARTAN | TERBUTALINE HEMISULFATE | D-LACTITOL MONOHYDRATE |
| HOMOSALATE | TETRACAINE | LEVOCARNITINE |
| | HYDROCHLORIDE | |
| SALICYLANILIDE | TETRACYCLINE | LOBENDAZOLE |
| | HYDROCHLORIDE | |
| PROPOFOL | TETRAHYDROZOLINE | METHYLENE BLUE |
| | HYDROCHLORIDE | |
| GRISEOFULVIN | THIABENDAZOLE | METHYLATROPINE NITRATE |
| BENAZEPRIL | THIMEROSAL | NITHIAMIDE |
| HYDROCHLORIDE | | |
| VALSARTAN | THIOGUANINE | PRALIDOXIME CHLORIDE |
| SALSALATE | THIORIDAZINE | PREDNISOLONE |
| | HYDROCHLORIDE | HEMISUCCINATE |
| HYDROCORTISONE | THIOTHIXENE | PYRIDOXINE |
| RIFAXIMIN | TIMOLOL MALEATE | RIMANTADINE |
| | | HYDROCHLORIDE |
| CANRENONE | TOBRAMYCIN | SULFISOXAZOLE ACETYL |
| MODAFINIL | TOLAZOLINE | TAURINE |
| | HYDROCHLORIDE | |
| CLIOQUINOL | TOLBUTAMIDE | THIAMINE |
| RANOLAZINE | TRANYLCYPROMINE | TRICLOSAN |
| | SULFATE | |
| DANTHRON | TRIACETIN | TRIMETHADIONE |
| ACEDAPSONE | TRIAMCINOLONE | ZINC UNDECYLENATE |
| ATOMOXETINE | TRIAMCINOLONE | UNDECYLENIC ACID |
| HYDROCHLORIDE | ACETONIDE | |
| DESOXYCORTICOSTERONE | TRIAMCINOLONE | CLINDAMYCIN PALMITATE |
| ACETATE | DIACETATE | HYDROCHLORIDE |
| TRAMADOL HYDROCHLORIDE | TRIAMTERENE | CEFONICID SODIUM |
| TERBINAFINE | TRICHLORMETHIAZIDE | IFOSFAMIDE |
| HYDROCHLORIDE | | |
| TOPIRAMATE | TRIFLUOPERAZINE | NETILMICIN SULFATE |
| | HYDROCHLORIDE | |
| GEMIFLOXACIN MESYLATE | TRIHEXYPHENIDYL | DOXORUBICIN |
| | HYDROCHLORIDE | |
| PRAVASTATIN SODIUM | TRIMEPRAZINE TARTRATE | METHYSERGIDE MALEATE |
| LEVALBUTEROL | TRIMETHOPRIM | SOLIFENACIN |
| HYDROCHLORIDE | | |
| METFORMIN | TRIOXSALEN | ACEPROMAZINE MALEATE |
| HYDROCHLORIDE | | |
| PREGABALIN | TRIPELENNAMINE CITRATE | BIPERIDEN |
| PHENOXYBENZAMINE | TRIPROLIDINE | DEXCHLORPHENIRAMINE |
| HYDROCHLORIDE | HYDROCHLORIDE | MALEATE |
| TOPOTECAN | TROPICAMIDE | DILOXANIDE FUROATE |
| HYDROCHLORIDE | | |
| PINACIDIL | TRYPTOPHAN | ETIDRONATE DISODIUM |
| VERAPAMIL | TUAMINOHEPTANE SULFATE | NATAMYCIN |
| HYDROCHLORIDE | | |
| PANTOPRAZOLE | TYROTHRICIN | NORGESTIMATE |
| LOPERAMIDE | UREA | TERAZOSIN |
| HYDROCHLORIDE | | HYDROCHLORIDE |
| PODOFILOX | URSODIOL | TIOCONAZOLE |
| LEVODOPA | VALPROATE SODIUM | ERGOTAMINE TARTRATE |
| RUTOSIDE (rutin) | VANCOMYCIN | ANAGRELIDE |
| | HYDROCHLORIDE | HYDROCHLORIDE |
| ZOMEPIRAC SODIUM | VIDARABINE | ETOMIDATE |
| SPARTEINE SULFATE | WARFARIN | LAMOTRIGINE |
| TESTOSTERONE PROPIONATE | XYLOMETAZOLINE | RALOXIFENE |
| | HYDROCHLORIDE | HYDROCHLORIDE |
| METHIMAZOLE | ACETARSOL | CEFPODOXIME PROXETIL |
| ENILCONAZOLE | MERBROMIN | TADALAFIL |
| FIROCOXIB | PHENACETIN | AMINOPENTAMIDE |
| LINDANE | PHENYLMERCURIC ACETATE | ARSANILIC ACID |
| ACRISORCIN | SULFANILAMIDE | PANTHENOL |
| PHENYL AMINOSALICYLATE | AZELAIC ACID | PHENTERMINE |
| TESTOSTERONE | PHENETHICILLIN POTASSIUM | TRIENTINE HYDROCHLORIDE |
| SANGUINARINE SULFATE | THEOBROMINE | TICLOPIDINE |
| | | HYDROCHLORIDE |
| alpha-TOCHOPHEROL | STRYCHNINE | TICARCILLIN DISODIUM |
| alpha-TOCHOPHERYL | ACONITINE | TETRAMIZOLE |
| ACETATE | | HYDROCHLORIDE |
| DACTINOMYCIN | YOHIMBINE | TOLTRAZURIL |
| | HYDROCHLORIDE | |
| MITOMYCIN C | ADENOSINE PHOSPHATE | TOREMIPHENE CITRATE |
| DICHLORVOS | KETOTIFEN FUMARATE | ROLIPRAM |
| TEMEFOS | BETAHISTINE | ROLITETRACYCLINE |
| | HYDROCHLORIDE | |
| MITOTANE | MOLSIDOMINE | PIPAMPERONE |
| IVERMECTIN | MYCOPHENOLIC ACID | PANCURONIUM BROMIDE |
| SODIUM NITROPRUSSIDE | OLEANDOMYCIN | FUMAZENIL |
| | PHOSPHATE | |
| SODIUM OXYBATE | OUABAIN | ALTRENOGEST |
| ETHYL PARABEN | ALBUTEROL (+/−) | BISOPROLOL FUMARATE |
| COUMARIN | ARECOLINE HYDROBROMIDE | FLUDARABINE PHOSPHATE |
| ACETAMINOPHEN | CAPTOPRIL | MUPIROCIN |
| ACETAZOLAMIDE | CIMETIDINE | TEICOPLANIN [A(2-1) shown] |
| ACETOHYDROXAMIC ACID | CLOZAPINE | EPIRUBICIN |
| | | HYDROCHLORIDE |
| ACETYLCHOLINE | HYDRASTINE (1R,9S) | VECURONIUM BROMIDE |
| ACETYLCYSTEINE | LIDOCAINE | ALISKIREN HEMIFUMARATE |
| | HYDROCHLORIDE | |
| ADENOSINE | PHENTOLAMINE | ACAMPROSATE CALCIUM |
| | HYDROCHLORIDE | |
| ALLOPURINOL | BUTAMBEN | PREDNISOLONE SODIUM |
| | | PHOSPHATE |
| ALVERINE CITRATE | CEFACLOR | PREGNENOLONE SUCCINATE |
| AMANTADINE | IODIPAMIDE | DARIFENACIN |
| HYDROCHLORIDE | | HYDROBROMIDE |
| AMIKACIN SULFATE | LIOTHYRONINE | DESOXYMETASONE |
| AMILORIDE HYDROCHLORIDE | ALLANTOIN | BETAMETHASONE ACETATE |
| AMINOCAPROIC ACID | ALTHIAZIDE | ERYTHROSINE SODIUM |
| AMINOGLUTETHIMIDE | ADENINE | ISOFLUPREDNONE ACETATE |
| AMINOSALICYLATE SODIUM | AMINACRINE | BETAMETHAZONE SODIUM |
| | | PHOSPHATE |
| AMITRIPTYLINE | BEKANAMYCIN SULFATE | MELENGESTROL ACETATE |
| HYDROCHLORIDE | | |
| AMODIAQUINE | BUDESONIDE | PHTHALYLSULFACETAMIDE |
| DIHYDROCHLORIDE | | |
| AMOXICILLIN | BRUCINE | TRICHLORFON |
| AMPHOTERICIN B | CANRENOIC ACID, | BEPHENIUM |
| | POTASSIUM SALT | HYDROXYNAPTHOATE |
| AMPICILLIN SODIUM | CHENODIOL | DIPERODON |
| | | HYDROCHLORIDE |
| AMPROLIUM | CHOLECALCIFEROL | DIATRIZOIC ACID |
| ANTAZOLINE PHOSPHATE | CINCHONIDINE | PANTOTHENIC ACID(d) Na salt |
| ANTHRALIN | CINCHONINE | DESONIDE |
| ANTIPYRINE | COENZYME B12 | GLYCOPYRROLATE |
| APOMORPHINE | CHOLESTEROL | ITRACONAZOLE |
| HYDROCHLORIDE | | |
| ASPIRIN | PIPERINE | OCTISALATE |
| ATROPINE SULFATE | ETOPOSIDE | RIBOFLAVIN 5-PHOSPHATE |
| | | SODIUM |
| AUROTHIOGLUCOSE | DEHYDROCHOLIC ACID | SELEGILINE |
| | | HYDROCHLORIDE |
| AZATHIOPRINE | FLUMEQUINE | CEFTAZIDIME |
| BACITRACIN | FLUNARIZINE | GABAPENTIN |
| | HYDROCHLORIDE | |
| BACLOFEN | FLUPHENAZINE | ELETRIPTAN |
| | HYDROCHLORIDE | HYDROBROMIDE |
| BECLOMETHASONE | FLUTAMIDE | ARIPIPRAZOLE |
| DIPROPIONATE | | |
| BENSERAZIDE | DROPERIDOL | ZILEUTON |
| HYDROCHLORIDE | | |
| BENZETHONIUM CHLORIDE | FAMOTIDINE | METHYLPHENIDATE |
| | | HYDROCHLORIDE |
| BENZOCAINE | ETODOLAC | RABEPRAZOLE SODIUM |
| BENZTHIAZIDE | FENOTEROL | RISEDRONATE SODIUM |
| | HYDROBROMIDE | HYDRATE |
| beta-CAROTENE | FENBUFEN | SUCRALOSE |
| BETAMETHASONE | MEBEVERINE | COLISTIN SULFATE |
| | HYDROCHLORIDE | |
| BETHANECHOL CHLORIDE | ACECLIDINE | ARSENIC TRIOXIDE |
| BISACODYL | CAPSAICIN | CLONAZEPAM |
| BITHIONATE SODIUM | FAMPRIDINE | BENZBROMARONE |
| BROMOCRIPTINE MESYLATE | NICERGOLINE | BROMPERIDOL |
| BUSULFAN | SPIPERONE | CYPROHEPTADINE |
| | | HYDROCHLORIDE |
| CAFFEINE | ERYTHROMYCIN ESTOLATE | CLOFAZIMINE |
| CAMPHOR (1R) | ESTRADIOL PROPIONATE | BENZYDAMINE |
| | | HYDROCHLORIDE |
| CAPREOMYCIN SULFATE | ESTRADIOL BENZOATE | DOXAZOSIN MESYLATE |
| CARBACHOL | RETINOL | ISOETHARINE MESYLATE |
| CARBAMAZEPINE | ISOTRETINON | FLORFENICOL |
| CARBENICILLIN DISODIUM | MESNA | ETHYNODIOL DIACETATE |
| CARBINOXAMINE MALEATE | TRETINON | ORNIDAZOLE |
| CARISOPRODOL | BRETYLIUM TOSYLATE | OXANTEL PAMOATE |
| CEFADROXIL | FOSCARNET SODIUM | PROTRYPTYLINE |
| | | HYDROCHLORIDE |
| CEFOTAXIME SODIUM | CEFSULODIN SODIUM | PHYTONADIONE |
| CEPHALOTHIN SODIUM | FOSFOMYCIN CALCIUM | DENATONIUM BENZOATE |
| CEPHAPIRIN SODIUM | CEFAMANDOLE NAFATE | MESALAMINE |
| CEPHRADINE | LIOTHYRONINE (L-isomer) | ETHAMIVAN |
| | SODIUM | |
| CETYLPYRIDINIUM CHLORIDE | ALRESTATIN | AZTREONAM |
| CHLORAMBUCIL | PROADIFEN | TYLOXAPOL |
| | HYDROCHLORIDE | |
| CHLORAMPHENICOL | CARBOPLATIN | THIAMYLAL SODIUM |
| PALMITATE | | |
| CHLORAMPHENICOL | CISPLATIN | CHLORDIAZEPOXIDE |
| HEMISUCCINATE | | |
| CHLORAMPHENICOL | ZIDOVUDINE [AZT] | ASTEMIZOLE |
| CHLORCYCLIZINE | AZACITIDINE | ACECAINIDE |
| HYDROCHLORIDE | | HYDROCHLORIDE |
| CHLORHEXIDINE | CYCLOHEXIMIDE | FLUROTHYL |
| CHLOROCRESOL | TINIDAZOLE | ALPRENOLOL |
| CHLOROQUINE DIPHOSPHATE | CARBIDOPA | AMIODARONE |
| | | HYDROCHLORIDE |
| CHLOROTHIAZIDE | ETHOSUXIMIDE | BUSPIRONE |
| | | HYDROCHLORIDE |
| CHLOROTRIANISENE | PIPERIDOLATE | LOXAPINE SUCCINATE |
| | HYDROCHLORIDE | |
| CHLOROXYLENOL | ANISINDIONE | DIAZOXIDE |
| CHLORPHENIRAMINE (S) | CYCLOSPORINE | DILTIAZEM HYDROCHLORIDE |
| MALEATE | | |
| CHLORPROMAZINE | ASCORBIC ACID | GLYBURIDE |
| CHLORPROPAMIDE | MENADIONE | MIANSERIN |
| | | HYDROCHLORIDE |
| CHLORTETRACYCLINE | SALICIN | VESAMICOL |
| HYDROCHLORIDE | | HYDROCHLORIDE |
| CHLORTHALIDONE | MONENSIN SODIUM (monensin | NIZATIDINE |
| | A is shown) | |
| CHLORZOXAZONE | ABAMECTIN | PENTYLENETETRAZOL |
| CICLOPIROX OLAMINE | BENZOIC ACID | NICOTINE DITARTRATE |
| CINOXACIN | BENZYL BENZOATE | TACRINE HYDROCHLORIDE |
| CLEMASTINE | BENZOYL PEROXIDE | DIMERCAPROL |
| CLIDINIUM BROMIDE | BETAINE HYDROCHLORIDE | METOLAZONE |
| CLINDAMYCIN | BIOTIN | AMOXAPINE |
| HYDROCHLORIDE | | |
| CLOMIPHENE CITRATE | AKLOMIDE | BUTYL PARABEN |
| CLONIDINE HYDROCHLORIDE | NICOTINYL ALCOHOL | DECAMETHONIUM BROMIDE |
| | TARTRATE | |
| CLOTRIMAZOLE | FLOXURIDINE | CARBADOX |
| CLOXACILLIN SODIUM | ALTRETAMINE | ENROFLOXACIN |
| CLOXYQUIN | AMINOHIPPURIC ACID | DEXPANTHENOL |
| COLCHICINE | MEFLOQUINE | NONOXYNOL-9 |
| COLISTIMETHATE SODIUM | ADIPHENINE | DOCOSANOL |
| | HYDROCHLORIDE | |
| CORTISONE ACETATE | QUINAPRIL | OCTODRINE |
| | HYDROCHLORIDE | |
| COTININE | AMIFOSTINE | ANIRACETAM |
| CRESOL | AMIPRILOSE | PENTOXIFYLLINE |
| CROMOLYN SODIUM | TIAPRIDE HYDROCHLORIDE | AZTREONAM |
| CYCLIZINE | BACAMPICILLIN | CEFAZOLIN SODIUM |
| | HYDROCHLORIDE | |
| CYCLOPENTOLATE | CYPROTERONE ACETATE | TUBOCURARINE CHLORIDE |
| HYDROCHLORIDE | | |
| CYCLOPHOSPHAMIDE | CYTARABINE | TOLMETIN SODIUM |
| HYDRATE | | |
| CYCLOSERINE | DACARBAZINE | BENDROFLUMETHIAZIDE |

TABLE 8
Top 200 Brand Name Drugs 2008

| | |
|---|---|
| 1 | Lipitor |
| 2 | Nexium |
| 3 | Plavix |
| 4 | Advair Diskus |
| 5 | Prevacid |
| 6 | Seroquel |
| 7 | Singulair |
| 8 | Effexor XR |
| 9 | OxyContin |
| 10 | Actos |
| 11 | Lexapro |
| 12 | Abilify |
| 13 | Topamax |
| 14 | Cymbalta |
| 15 | Zyprexa |
| 16 | Valtrex |
| 17 | Crestor |
| 18 | Vytorin |
| 19 | Lamictal |
| 20 | Celebrex |
| 21 | Lantus |
| 22 | Levaquin |
| 23 | Adderall XR |
| 24 | Lyrica |
| 25 | Diovan |
| 26 | Tricor |
| 27 | Flomax |
| 28 | Risperdal |
| 29 | Diovan HCT |
| 30 | Zetia |
| 31 | Aricept |
| 32 | Spiriva |
| 33 | Concerta |
| 34 | Aciphex |
| 35 | Imitrex Oral |
| 36 | Lidoderm |
| 37 | Keppra |
| 38 | Viagra |
| 39 | Atripla |
| 40 | Lovenox |
| 41 | Januvia |
| 42 | Nasonex |
| 43 | Ambien CR |
| 44 | Provigil |
| 45 | Geodon Oral |
| 46 | Truvada |
| 47 | Lunesta |
| 48 | Enbrel |
| 49 | Actonel |
| 50 | CellCept |
| 51 | Humalog |
| 52 | Detrol LA |
| 53 | Depakote ER |
| 54 | Cozaar |
| 55 | Pulmicort Respules |
| 56 | Niaspan |
| 57 | Wellbutrin XL |
| 58 | Chantix |
| 59 | Budeprion XL |
| 60 | Byetta |
| 61 | Yaz |
| 62 | Prograf |
| 63 | Namenda |
| 64 | Arimidex |
| 65 | Combivent |
| 66 | Cialis |
| 67 | Flovent HFA |
| 68 | Protonix |
| 69 | Premarin Tabs |
| 70 | Suboxone Hyzaar |
| 71 | Hyzaar |
| 72 | ProAir HFA |
| 73 | Reyataz |
| 74 | Benicar HCT |
| 75 | Synthroid |
| 76 | Avandia |
| 77 | Boniva |
| 78 | Strattera |
| 79 | Polymagma Plain |
| 80 | Skelaxin |
| 81 | Evista |
| 82 | Asacol |
| 83 | Depakote |
| 84 | Xalatan |
| 85 | Humira |
| 86 | Benicar |
| 87 | Gleevec |
| 88 | AndroGel |
| 89 | Enbrel Sureclick |
| 90 | Avelox |
| 91 | Fantanyl Oral Citra |
| 92 | Lovaz |
| 93 | RenaGel |
| 94 | Avapro |
| 95 | Humira Pen |
| 96 | Vyvanse |
| 97 | Kaletra |
| 98 | Xopenex |
| 99 | Copaxone |
| 100 | Avodart |
| 101 | Femara |
| 102 | Avalide |
| 103 | Ortho TriCyclen |
| | Lo |
| 104 | Sensipar |
| 105 | Aldara |
| 106 | NovoLog Mix |
| 107 | Restasis |
| 108 | Mirapex |
| 109 | Yasmin 28 |
| 110 | Solodyn |
| 111 | Lantus SoloSTAR |
| 112 | Norvir |
| 113 | Focalin XR |
| 114 | Actoplus Met |
| 115 | Vesicare |
| 116 | Forteo |
| 117 | Allegra-D |
| 118 | Procrit. |
| 119 | Nasacort AQ |
| 120 | Tarceva |
| 121 | Combivir |
| 122 | Tamiflu |
| 123 | Avonex |
| 124 | NuvaRing |
| 125 | Coreg CR |
| 126 | Epzicom |
| 127 | Levemir |
| 128 | Duragesic |
| 129 | Risperdal |
| | Consta |
| 130 | Zyvox |
| 131 | Tussionex |
| 132 | Invega |
| 133 | Fosamax |
| 134 | Kadian |
| 135 | Levitra |
| 136 | Differin |
| 137 | Astelin |
| 138 | Lumigan |
| 139 | Symbicort |
| 140 | Janumet |
| 141 | Xeloda |
| 142 | Clarinex |
| 143 | Proventil |
| | HFA |
| 144 | Humalog Mix |
| | 75/25 Pn |
| 145 | BenzaClin |
| 146 | Vigamox |
| 147 | Foxamax Plus D |
| 148 | Maxalt |
| 149 | Cosopt |
| 150 | Requip |
| 151 | Relpax\ |
| 152 | Patanol |
| 153 | Casodex |
| 154 | Welchol |
| 155 | Ciprodex Otic |
| 156 | Viread |
| 157 | Catapres-TTS |
| 158 | Loestrin 24 Fe |
| 159 | Thalomid |
| 160 | Alphagan P |
| 161 | Endocet |
| 162 | Revlimid |
| 163 | Avandamet |
| 164 | Maxalt MLT |
| 165 | Altace |
| 166 | Budeprion SR |
| 167 | Pegasys |
| 168 | Ultram ER |
| 169 | Fentora |
| 170 | Asmanex |
| 171 | Rhinocort Aqua |
| 172 | Temodar |
| 173 | Micardis HCT |
| 174 | Sotret |
| 175 | Trizivir |
| 176 | Enablex |
| 177 | Isentress |
| 178 | TobraDex |
| 179 | Trileptal |
| 180 | Sustiva |
| 181 | Amitiza |
| 182 | Micardis |
| 183 | Zovirax |
| | Topical |
| 184 | Ocella |
| 185 | Propecia |
| 186 | Taclonex |
| 187 | Actiq |
| 188 | Valcyte |
| 189 | Klor-Con |
| 190 | Atacand |
| 191 | Doryx |
| 192 | Veramyst |
| 193 | Avinza |
| 194 | Allegra-D 24 |
| | Hour |
| 195 | Opana ER |
| 196 | Zomig |
| 197 | Humulin 70/30 |
| 198 | Prempro |
| 199 | Humulin N |
| 200 | Xopenex HFA |

TABLE 9
Exemplary Scaffolds

| Compound # | Formula |
|---|---|
| 1 | 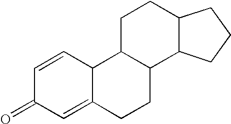 |
| 2 | 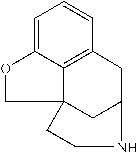 |
| 3 | 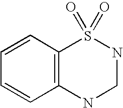 |
| 4 | 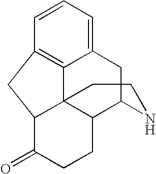 |
| 5 | 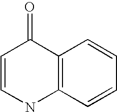 |
| 6 | 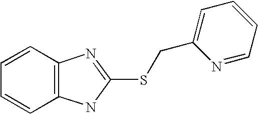 |
| 7 | 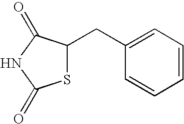 |
| 8 | 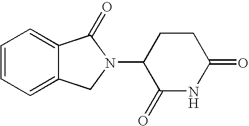 |
| 9 | 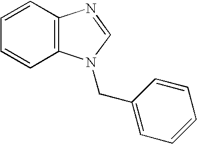 |
| 10 | 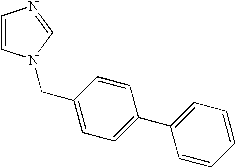 |
| 11 | 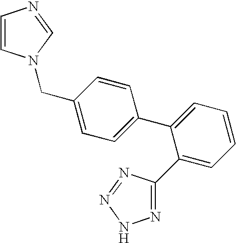 |

1. A method comprising:
(a) passing a biological sample comprising a target protein and optionally a non-target protein over a column, the column comprising an affinity resin for the target protein, the affinity resin comprising a resin conjugated or covalently attached to a first chemical compound that binds to the target protein;
(b) washing the column and removing proteins that are not bound to the affinity resin;
(c) eluting proteins from the column that are bound to the affinity resin by passing a solution comprising a second chemical compound over the column; and
(d) identifying proteins in the eluate, thereby obtaining a proteomic profile for the second chemical compound.

2. The method of claim 1 further comprising:
(e) comparing the proteomic profile of the second chemical compound to a proteomic profile of the first chemical compound obtained by eluting proteins from the column that are bound to the affinity resin by passing a solution comprising the first chemical compound over the column.

3. The method of claim 1, wherein the second chemical compound is a derivative or analog of the first chemical compound and binds to the target protein.

4. The method of claim 1, wherein the first chemical compound and the second chemical compound are selected from Tables 6-9.

5. The method of claim 1, wherein identifying the proteins in the eluates comprises performing sodium dodecyl sulfate (SDS) polyacrylamide gel electrophoresis (PAGE).

6. The method of claim 5, further comprising measuring intensities of bands in the gel by electronically scanning the gels and performing densitometry analysis.

7. The method of claim 1, wherein proteins in the eluate are identified by performing tandem mass spectrometry (MS) analysis.

8. The method claim 5, further comprising excising separate bands from the gels and performing tandem MS analysis each excised band.

9. The method of claim 1, wherein the first chemical compound is DMP543 or an analog or derivative thereof that inhibits KCNQ (Kv7) channel activity.

10. The method of claim 9, wherein DMP543 is conjugated or covalently attached to the resin as follows:

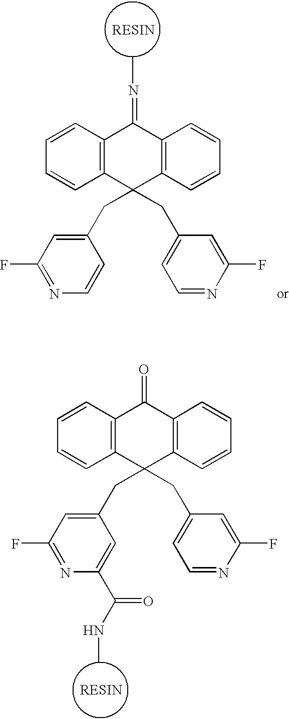

11. The method of claim 1, wherein the first chemical compound is XE991 or an analog or derivative thereof that inhibits KCNQ (Kv7) channel activity.

12. The method of claim 11, wherein XE991 is conjugated or covalently attached to the resin as follows:

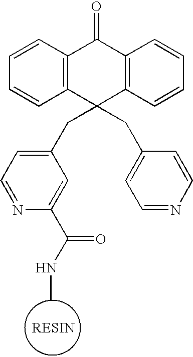

13. The method of claim 1, wherein the first chemical compound is linopirdine or an analog or derivative thereof that inhibits KCNQ (Kv7) channel activity.

14. The method of claim 13, wherein linopirdine is conjugated or covalently attached to the resin as follows:

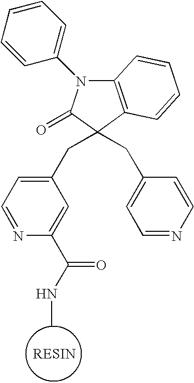

15. The method of claim 1, wherein the first chemical compound is CRAA.

16. The method of claim 1, wherein the first chemical compound is glitazone.

17. The method of claim 1, wherein the biological sample is selected from a neurological tissue sample, a liver tissue sample, a heart tissue sample, and a kidney tissue sample.

18. A method comprising:
(a) passing a biological sample comprising proteins over columns comprising a chemical-resin library, wherein each column comprises a separate member of the chemical-resin library and the chemical-resin library comprises a separate chemical compound conjugated to a resin;
(b) washing each column to remove any non-bound proteins;
(c) eluting any bound proteins from each column; and
(d) identifying proteins in the eluates, thereby generating a proteomic profile for each column, and optionally comparing the proteomic profiles for two or more columns.

19. A method comprising:
(a) passing a biological sample comprising a target protein and a non-target protein over a first column, the first column comprising an affinity resin for the target protein, the affinity resin comprising a resin conjugated or covalently attached to a first chemical compound that binds to the target protein;
(b) washing the first column and removing proteins that are not bound to the affinity resin;
(c) eluting proteins from the first column that are bound to the affinity resin;
(d) identifying proteins in the eluate including the target protein and optionally the non-target protein;
(e) passing the biological sample comprising the target protein and the non-target protein over a second column, the second column comprising an affinity resin for the target protein, the affinity resin comprising a resin conjugated or covalently attached to a second chemical compound that binds to the target protein;
(f) washing the second column and removing proteins that are not bound to the affinity resin;
(g) eluting proteins from the second column that are bound to the affinity resin; and
(h) identifying proteins in the eluate including the target protein and optionally the non-target protein.

20. The method of claim 19, wherein the second chemical compound is a derivative or analog of the first chemical compound that binds the target protein with an affinity no less than the first chemical compound and that binds the non-target protein with an affinity less than the first chemical compound.
